(19) United States
(12) Patent Application Publication
(10) Pub. No.: US 2008/0248008 A1
(43) Pub. Date: Oct. 9, 2008

(54) Inhibitors of Protein Kinase a Anchoring

(76) Inventors: Cathrine Rein Carlson, Oslo (NO); Kjetil Tasken, Rykkinn (NO); John Scott, Portland, OR (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/575,966

(22) PCT Filed: Sep. 26, 2005
(86) PCT No.: PCT/GB2005/003702

§ 371 Date: May 1, 2008

(30) Foreign Application Priority Data
Sep. 24, 2004 (GB) GB 0421355.9

(51) Int. Cl. A61K 35/12; C12N 9/12; C07K 16/18; C12N 15/11; C12P 21/04; A61P 37/00; C12Q 1/48; C12N 5/06; A61K 38/43; A61K 31/70

(52) U.S. Cl. 424/93.21; 435/194; 530/387.9; 536/23.2; 435/69.1; 435/375; 424/94.5; 514/44; 435/15

(57) ABSTRACT

A PKA I anchoring disrupting molecule or AKAP mimic, wherein said molecule or mimic is a polypeptide which comprises the following amino acid sequence: X₁ X₂ X₃ Y A X₄ X₅ L A X₆ X₇ X₈ I X₉ X₁₀ X₁₁ X₁₂ X₁₃ (sequence (1)) or a peptidomimetic or analogue thereof is provided. Also provided are antibodies to the molecule, nucleic acid molecules comprising a sequence encoding the molecule and pharmaceutical compositions. A method of altering the PKA type I signalling pathway in a cell by administration of the anchoring disruption molecule or AKAP mimic, in particular to treat immunosuppressive disorders, proliferative diseases or autoimmune diseases is also provided.

The present invention relates to anchoring disruption molecules and related molecules which modulate the function of cAMP dependent protein kinase A type I and their use to produce pharmaceutical preparations to treat or prevent diseases typified by elevated or reduced PKA I activity, such as immunosuppressive diseases. More specifically, the present invention provides inhibitors of binding between PKA I and A kinase anchoring proteins, (AKAPs) that normally serve to localise PKA I to specific regions of the cell. In particular said anchoring disruption molecules bind to the AKAP binding site of PKA type I. In the alternative, the invention provides AKAP mimics which bind to PKA I and which may also facilitate its identification, isolation or localization.

The immune system of mammals has evolved different strategies to defend the organism against the variety of potentially infectious agents. The ability to acquire specific and anamnestic responses against intruders relies on the adaptive immune system. The main players in the adaptive immune system are B and T lymphocytes, and the specific recognition of antigen by these cells is mediated by receptors with some degree of structural similarity, yet which are functionally very different. The different receptor specificities are made possible through somatic rearrangement of a limited number of genes and are clonally distributed. The main strategy of this system is to generate a nearly unlimited number of specificities to cover the recognition of almost any foreign antigen. Immunological memory is partly a result of clonal expansion of subsets of T and B cells reacting with a particular antigen, and enables the organism to respond more quickly at the second encounter with the same antigen.

Cell proliferation is used as a marker of immune activation. According to the clonal selection theory, exposure to antigen leads to activation of individual B and T cell clones with corresponding receptor specificities. However, the number of cells with affinity for a certain antigen is a small fraction of the total number of cells (approximately 0.001%). It is therefore crucial that the activated cells are capable of proliferation (clonal expansion) in order to generate an adequate immune response. Thus, proliferation is a very important feature characterizing lymphocyte function and allowing immune activation. In in vitro experiments, it is possible to activate the entire population of isolated T lymphocytes by using antibodies directed against the antigen receptor complex (TCR/CD3). This will mimic the in vivo situation in which T cells are immunoactivated to clonal expansion through the antigen receptor. It is known that T cell proliferation is inhibited through the cAMP signalling pathway.

Cyclic AMP-dependent protein kinase (PKA) is an enzyme present in all cells. Hormones and neurotransmitters binding to specific receptors stimulate the generation of the second messenger 3′,5′-cyclic adenosine monophosphate (cAMP). Cyclic AMP is one of the most common and versatile second messengers. The best characterized and major downstream effector mechanism whereby cAMP exerts its effects involves binding to and activating PKA. PKA is a serine/threonine protein kinase which phosphorylates a number of different proteins within the cell, and thereby regulates their activity. It is known that PKA regulates a vast variety of cellular processes such as metabolism, proliferation, differentiation and regulation of gene transcription.

The great diversity of cellular processes mediated by cAMP and PKA strongly suggests that there exists mechanisms that provide the required sensitivity and specificity of the effector pathway to ensure that rapid and precise signalling processes take place. Specificity can be achieved by tissue- and cell-type specific expression of PKA isoforms with different biochemical properties. However, targeting of PKA isoforms by A-kinase anchoring proteins (AKAPs) provides a higher level of specificity to the signalling process by localizing PKA to defined subcellular sites in close proximity to the substrate. Anchoring of PKA by AKAPs may also tune the sensitivity of the signal pathway by recruiting PKA into multiprotein complexes that include phosphodiesterases and protein phosphatases as well as other signal proteins in addition to PKA (Michel and Scott, 2002, Ann. Rev. Pharmacol. Toxicol., 42, p 235-257).

PKA is made up of four different subunits, a regulatory (R) subunit dimer and two catalytic (C) subunits. Furthermore, two main classes of PKA isozymes, PKA type I and PKA type II (PKA I and PKA II, respectively) have been described. PKA I and PKA II can be distinguished by their R subunits, designated RI and RII. Isoforms of RI and RII are referred to as RIα, RIβ, RIIα and RIIβ. Moreover, the C subunits also exist as isoforms referred to as Cα, Cβ and Cγ. The different subunits may form multiple forms of PKA (isozymes) with potentially more than 18 different forms.

Activation occurs upon binding of cAMP to the R subunits followed by the release of the active catalytic subunit. PKA type II is mainly particulate and associated with AKAPs whereas PKA type I is both soluble and particulate although PKA type I anchoring has remained more elusive. However, PKA type I is present in the lipid raft fraction of the cell membrane and colocalizes with the TCR-CD3 complex upon T-cell activation (Skålhegg et al., 1994, Science, 263, p 84-87). Lipid rafts are specialised membrane domains enriched in certain lipids, cholesterol and proteins. Their primary function is believed to be in signalling transduction. In particular they are considered to be the site of recruitment for various signalling molecules crucial for T cell activation.

PKA is a key negative regulator of lymphocyte function. It has been shown that cAMP inhibits T lymphocyte proliferation induced through the T cell antigen receptor/CD3 complex (TCR/CD3). T cells express both PKA I and PKA II. However, only the selective activation of PKA I is sufficient to mediate the inhibitory effect of cAMP. In addition, it has been demonstrated that PKA I, but not PKA II, redistributes to, colocalizes with and inhibits signalling through antigen receptors on T and B cells and natural killer cells and regulates mitogenic responses in T and B cells and acute cytotoxic responses in NK cells.

PKA type I mediates an inhibitory effect on the T cell activation cascade that involves activation of C-terminal Src kinase (Csk) by phosphorylating residue S364 (Vang et al, 2001, J. Exp. Med., 193, p 497-507). Active Csk subsequently phosphorylates the C-terminal inhibitory tyrosine residue of the Src kinase Lck which reduces its activity and thereby acts as a negative regulator of TCR signalling. The processes which are involved are described in more detail in WO99/62315, which is incorporated herein by reference. Thus, PKA I serves as a key negative regulator of lymphocyte functions, e.g. mitogenic and cytotoxic responses initiated through antigen receptors. Modulation of normal immune responsiveness by activation of PKA type I is a negative feedback mechanism. Dysregulation of this system may lead to immunological overshoot or impaired immune functions.

The enzyme cyclooxygenase-2 (COX-2) is believed to increase prostaglandin PGE₂ production which in turn increases the levels of cAMP which activates the PKA signalling pathway. Recent studies have shown that intake of non-steroidal anti-inflammatory drugs (NSAIDs) that inhibit the enzymatic activity of COX reduce the risk of developing colorectal cancer. Further studies have shown that selective COX-2 inhibitors are associated with a decline in the incidence of colorectal cancer and reduced mortality rate. While COX-1 is ubiquitously expressed at low levels, COX-2 is expressed at high levels at sites of inflammation in response to a wide spectrum of growth factors and pro-inflammatory cytokines. The different classes of prostaglandins exert their effects by binding to G-coupled cell-surface receptors leading to changes in the cellular levels of cAMP and Ca²⁺ and regulate diverse physiological processes including reproductive function, kidney function, vascular tone and permeability in addition to immune function. cAMP-mediated immunosuppression thus is implicated in cancer and especially locally in solid tumours inhibiting the immune system's ability to fight the tumour. Furthermore cAMP immunosuppression may be contributing to the general immune deficiency of late stage cancer and sepsis.

Both primary and secondary immunodeficiencies cause an increased incidence of opportunistic infections and cancer, and are increasing causes of morbidity and mortality in all parts of the world. Human immunodeficiency virus (HIV) causes a chronic infection leading to severe dysfunction of the immune system with markedly increased incidence of a large number of infections and certain forms of malignancies (e.g. lymphoma and Kaposis' sarcoma). In many communities in the USA, HIV infection is the leading cause of death among “young” adults. In the developing world this problem is even larger.

Next to immunoglobulin (Ig) A deficiency, common variable immunodeficiency (CVI) is the most frequent type of primary immunodeficiency. This form of primary hypogammaglobulinaemia is characterized by the onset of immunodeficiency after the first two years of life, by severely decreased serum IgG levels and recurrent bacterial infections, particularly in the respiratory tract.

T cell dysfunction is the immunological hallmark of HIV infection. Defective lymphocyte cytokine production and impaired proliferative responses on stimulation are early signs of immunodeficiency in these patients, manifested even before a decline in CD4+ lymphocytes counts is observed.

B cell dysfunction with impaired antibody synthesis is the major immunological characteristic of CVI patients. However, the immunological abnormalities in CVI are not restricted to B cells, but often also involve T cell dysfunction, e.g. impaired proliferative responses on stimulation. The B cells in CVI patients are not necessarily intrinsically defective, and impaired T cell “help” may be of importance for the B cell defects in these patients. T cell dysfunction may also be of importance for certain clinical manifestations in these patients not necessarily related to defective antibody production, e.g. increased incidence of granulomata and malignancies.

In terms of current therapies, antiretroviral therapy is the main component in the treatment of HIV-infected patients. However, although potent antiretroviral combination therapy may markedly increase the CD4+ and CD8+ lymphocytes counts in HIV-infected patients, impaired T cell function seems to persist, as indicated in the observations made in Example 1, table I and 2B of WO98/48807. Thus, there is a need for immunomodulating agents in addition to antiretroviral therapy in these patients.

Immunoglobulin substitution is the main component in the treatment of CVI patients. However, this substitution therapy does not restore the defective T and B cell function. Furthermore, in some clinical complications, e.g. noncaseating granulomata and persistent viral infections, there is a need for therapy which may more directly enhance T cell function.

Although impaired T cell function is a well recognized immunological feature of both HIV infection and CVI, the exact molecular mechanism for this T cell impairment was not known. Therapeutic modalities directed against such intracellular defects are expected to be of major importance in the treatment of these patients and may have the potential to restore important immunological defects in HIV-infected patients and in patients with CVI.

Hofmann et al (Aids, Vol 7, p 659-664, 1993) and WO93/19766 have demonstrated that HIV-seropositive individuals without AIDS show a significant increase in intracellular cAMP levels and PKA activity in crude peripheral blood mononuclear cells (PBMC) from HIV-seropositive subjects. Examination of T cells was reported as data not shown and did not reach significance because of larger variability, probably induced by the T cell purification method. Their study further indicated that adenosine analogues such as 2′,5′ dideoxyadenosine (ddAdo) reduced cellular cAMP levels in PBMC and increased cell proliferation. This effect was, however, concentration dependent such that concentrations in the range of 6 ng/ml were effective and higher concentrations were suppressive or did not further inhibit cAMP levels. Similar effects were not demonstrated in T cells sampled from HIV patients. A simple concentration/response relationship was also not demonstrated. Purified T cells were used in their examples but these cells were sampled from healthy blood donors and purified by positive selection that may lead to premature T cell activation.

Cho-Chung et al in WO93/21929 have shown treatment applied to cancer cells by antagonising cAMP-dependent protein kinase, by using phosphorothioate derivatives of cAMP.

It has been established that activation of protein kinase A type I isozymes (and not type II isozymes) is necessary and sufficient to mediate cAMP-dependent inhibition of immune functions such as T and B cell proliferation induced through the antigen receptor or NK cell cytotoxicity mediated by specific NK receptors (Skålhegg et al, 1992, J. Biol. Chem., 267, p 15707; Skålhegg et al, 1994, Science, 263, p 84). Furthermore, protein kinase A type I redistributes to and colocalizes with the antigen receptor complex upon activation and capping of T and B cells (Skålhegg et al, 1994, supra). This anchoring of protein kinase A type I supports a role for this specific isozyme in modulation of immune responses mediated through receptors on lymphoid cells.

Furthermore, WO98/48809 shows that there is an increased activation of protein kinase A type I in T cells from patients with HIV infection or CVI. It is further demonstrated that activation of this isozyme of PKA leads to inhibition of immune function that can be reversed by selectively inhibiting the type I and not the type II isozymes of PKA. Based on these findings, compounds aimed at reversing the inappropriate activation of protein kinase A type I in immunodeficiencies (such as HIV, CVI) and thereby restoring T cell function and immune responsiveness were sought.

Further strategies to specifically increase T cell immune function and reverse T cell dysfunction in human immunodeficiency virus infected and common variable immunodeficiency patients by using suitable compounds interfering with the cAMP/PKA pathway in T cells were also investigated. Furthermore, as the role of PKA type I as an immune modulator is shared among all lymphoid cells (T cells, B cells, NK cells) disruption of the cAMP/PKA pathway is also expected to be relevant to B and NK cell function.

Appropriate mechanisms for improving T cell function are described in WO98/48809, which is incorporated herein by reference. The strategies relied on disruption of the cAMP-induced inhibition of T cell immune responses by abolishing PKA type I/RI signalling. Specifically, a number of specific mechanisms for disrupting the effects mediated by cAMP dependent protein kinase and hence stimulation of immune function were described. These included the use of PKA isozyme-specific cAMP antagonists, gene function knock out strategies, ribozymes, sequence-specific antisense oligonucleotides and the use of anchoring disrupting competitor peptides to displace protein kinase A type I from its anchoring with the antigen receptor complex. WO98/48809 showed that these entities all interfere with signalling through protein kinase A type I and that they could be used separately or in combination in order to target and abolish the inappropriate activation of protein kinase A type I.

In WO99/62315, the use of various mechanisms to affect phoshorylation of key components of the PKA type I signalling pathways and in so doing alleviate the inhibition of lymphocyte activation is described.

With regard to the use of anchoring disrupting peptides, specific cAMP-mediated effects at defined subcellular loci had been shown to be dependent on anchoring of PKA type II via hydrophobic interactions with an amphipathic helix domain in AKAPs in close proximity to substrates at that subcellular location. Disruption of anchoring by 22-amino acid competition peptides to the interaction domain, introduced by liposome mediated peptide transfer, had been shown to abolish isozyme-specific effects mediated by PKA type II. No similar effects had however been shown for PKA type I.

WO98/48809 then describes specific competitor peptides to displace protein kinase A type I from its anchoring with the antigen receptor complex. In doing so, protein kinase A type I function was inhibited by removal of the activated enzyme from substrates in the antigen receptor complex that are relevant for inhibition of immune function in T cells.

Anchoring disruptors for PKA type I and II have been described previously, examples of such anchoring disruptors include Ht31 (Carr et al., J. Biol. Chem, 266:14188-92, 1991; Rosenmund et. al., Nature, 368(6474):853-6, 1994), AKAP-IS (Alto et. al., Proc Natl Acad Sci USA. 100:4445-50, 2003), and PV38 (Burns-Hamuro et. al. Proc Natl Acad Sci USA. 100:4072-7, 2003). However, the problem with all these anchoring disruptors is that they have relatively low affinity, which makes cell based experiments and especially in vivo use, very problematic in the absence of a peptide delivery system. Furthermore, the previously described disruptors are relatively unspecific in their displacement of different isozymes of PKA which in practice means that they are not suitable for medical use.

Thus, alternative anchoring disrupting compounds are required which have higher affinity for PKA I. Preferably, these compound have affinities for PKA I which are sufficiently high for them to be used in vivo, and are selective for PKA I in that they are able to discriminate between PKA I and PKA II.

The present invention relates to anchoring disrupting molecules which represent a considerable improvement over previously reported disruptive peptides in terms of both affinity for PKA I and selectivity for PKA I over PKA II.

Surprisingly, the inventors have identified a family of molecules which comprise newly defined amino acid sequences which share the property of binding to PKA I with high affinity and selectivity and therefore act in a cell to prevent or enhance PKA I localising to its normal position or redirecting PKA I to another position. In this way the function of PKA I is modulated by affecting localisation or recruitment.

These anchoring disrupting molecules and AKAP mimics share a newly identified consensus sequence of amino acids, or a derivative of this sequence, and as a consequence of this, behave as if they were binding domains of AKAPs. They bind to the regulatory subunit of PKA I, termed PKA RI. These anchoring disrupting molecules have a higher and more specific affinity for PKA RI than naturally occurring AKAPs or prior art peptides. Furthermore, they have a higher specificity for PKA RI than for PKA RII. As a result of these properties they are suitable drug candidates to modulate the cAMP-PKA I signalling pathway in a selective manner. The molecules of the invention may be used to disrupt signalling by acting as inhibitors of PKA I:AKAP binding or may act as mimics of that binding e.g. to achieve the PKA localization required for signalling.

In a first aspect therefore, the invention provides a PKA I anchoring disrupting molecule or AKAP mimic, wherein said molecule or mimic is a polypeptide which comprises the following amino acid sequence:

(sequence (1))

X₁ X₂ X₃ Y A X₄ X₅ L A X₆ X₇ X₈ I X₉ X₁₀ X₁₁ X₁₂
X₁₃

wherein
X₁ is L, C, I, Y, V, W or F (preferably L, C, I or F, especially preferably L);
X₂ is K, R, H, E, D, C, V, A, I, Q, S, T or L (preferably K, R, D or E);
X₃ is Q, D, E, A, S, I, F, K, R, L, M, T, G, N, W or V (preferably Q, D, E, A, S, I, V, especially preferably Q);
X₄ is N, D, E, S, A, M, K, R, G, T, W or Q (preferably N, D, E or S);
X₅ is Q, D, E, M, F, I, S, K, R, C, W or Y (preferably Q, D, E, F, I or M);
X₆ is S, D, M, N, E, I, A, R, F, H, W, K, L, Y, Q or G (preferably S, M, E or D);
X₇ is Q, D, E, I, K, R, T, V, F, N, S, L, W or M (preferably Q, M, E or D);

### X₈ is I, A, S, L, D, B or V;

X₉ is K, C, D, E, R, A, M, T, W, H, Q or Y (preferably K or R);
X₁₀ is E, D, R, Q or K (preferably E or D);
X₁₁ is A, C, I, F, L, G, H or V (preferably A);
X₁₂ is T, C, L, F, I, V, M, K, R or W (preferably T, L or W); and
X₁₃ is E, D, N, V, Y, K, A, F, G, H, I, Q, L, M, R, S, T or W (preferably E, D, R, K or W),
or a peptidomimetic or analogue thereof.

Additionally X₅ may be L or T, X₉ may be L or S.

By “anchoring disruption molecule” it is meant a molecule which binds to, or associates with PKA RI and thereby is capable of preventing the normal PKA RI-AKAP interaction taking place. As a result, when used in cells, PKA RI does not become localised to the lipid rafts and cannot function to signal in the PKA I signalling pathway. The presence of an anchoring disruption molecule in a cell thus alters, preferably reduces, PKAI signalling.

Anchoring disruption molecules of the invention thus have the ability to interact with PKA RI, in a reversible or irreversible manner. In other words the anchoring disruption molecule associates with PKA RI. The structure of the anchoring disruption molecules of the invention are such that they bind to or associate with PKA RI at the site at which PKA RI would normally interact with an AKAP molecule. This site has been defined as residues 1 to 90, particularly 1-70 and more specifically 12-61 or 16-24 of the PKA RI amino acid sequence. Thus the anchoring disruption molecule associates with, preferably binds to, a molecule comprising amino acid residues 1 to 90, 1-70, 12-61 or 16-24 of PKA RI.

The anchoring disruption molecule may thus be considered as being a direct inhibitor of PKA RI anchoring, in that it associates with or binds to PKA RI and blocks the interaction of PKA RI with AKAPs e.g. sterically by occupying the AKAP binding site on PKA RI. These molecules therefore act as artificial PKA RI binding sites and compete with endogenous AKAPs to bind to PKA RI. Anchoring disruption molecules may thus be seen as competitors of PKA RI-AKAP binding as both endogenous AKAP and anchoring disruption molecules will bind to PKA RI. PKA RI is therefore prevented from interacting with endogenous AKAPs in the presence of an anchoring disruption molecule and its normal localisation in the lipid rafts is disrupted. Binding via an AKAP may be disrupted for example in mitochondria (which occurs e.g. via Pap7 or D-AKAP1), in ER (e.g. via D-AKAP1), in sperm (e.g. via AKAP82), in centrosomes, in membranes colocalized with ion channels, in the nucleus, in nuclear membranes in cytoplasm, on vesicles and peroxisomes, in the Golgi, with actin or microtubules or at other sites where PKA type I associates with AKAPs. Various targets to which unique targeting domains of the different AKAPS are directed have been identified (Taskén & Aandahl, 2003, Physiol. Rev., 84, p 137-167). As a consequence of the anchoring disruption molecule being designed to have a higher affinity for PKA RI than endogenous AKAPs, it is possible to displace PKA RI from AKAPs to which it is already bound when the anchoring disruption molecule is administered.

The ability of a molecule to act as an anchoring disruption molecule is thus dependent on its ability to selectively associate with or bind to PKA RI with high affinity. As referred to herein “binding” refers to the interaction or association of a least two moieties in a reversible or irreversible reaction, wherein said binding is preferably specific and selective. Specific binding refers to binding which relies on specific features of the molecules involved to achieve binding, i.e. does not occur when a non-specific molecule is used (i.e. shows significant binding relative to background levels) and is selective insofar as binding occurs between those partners in preference to binding to any of the majority of other molecules which may be present, particularly PKA RII, as described hereinafter.

The binding or association of the anchoring disruption molecule serves to reduce or inhibit binding of PKA RI to an AKAP. Reduced binding in this sense refers to a decrease in binding e.g. as manifest by reduced affinity for one another and/or an increased concentration of one of this binding pair required to achieve binding. Reduction includes a slight decrease as well as absolute abrogation of specific binding. A total reduction of specific binding is considered to equate to a prevention of binding. “Inhibited” binding refers to adversely affecting (e.g. by competitive interference) the binding of the binding partners by use of an anchoring disruption molecule which serves to reduce the partners' binding.

A reduction in binding or inhibition of binding may be assessed by any appropriate technique which directly or indirectly measures binding between the binding partners. Thus relative affinity may be assessed, or indirect effects reliant on that binding may be assessed. Thus for example, the binding of the 2 binding partners in isolated form may be assessed in the presence of the anchoring disruption molecule or AKAP mimic. Alternatively tests may be conducted in which the signalling achieved by the PKA type I pathway is examined or by assessing disrupted or redirected localization as evident from the presence of one or more binding partners in biochemical subcellular fractionation such as lipid raft purification or by immunofluorescent staining and epifluorescence microscopy. Anchoring disruption molecules or AKAP mimics may be labelled to follow such processes.

As mentioned above, the anchoring disruption molecules of the invention have a high affinity for PKA RI. The anchoring disruption molecules in general have a higher affinity for PKA RI than endogenous AKAPS. Preferably the anchoring disruption molecule has both a higher affinity and specificity for PKA RI than endogenous AKAPs present in the cell.

The ability of a peptide or peptidomimetic to act as an anchoring disruption molecule, i.e. to bind to PKA RI and the strength of binding (the affinity of binding) can be measured in a number of different ways, which are standard in the art and would be considered routine by the person skilled in the art. Examples of such methods include overlay or far western techniques, using radiolabelled RI subunit (see Example 1), measuring dissociation constants (K_{D}, Example 2) or coimmunoprecipitation techniques (Example 4). These techniques may also be used to determine whether a potential anchoring disruption molecule has the requisite level of selectivity or specificity.

Alternatively, binding may be detected or measured based on the functional effects of the binding as described above for measuring the extent of binding between PKA RI and an AKAP, e.g. by measuring the amount of PKA I signalling (e.g. measuring a downstream signal or effect such as IL-2 release or T-cell proliferation).

The anchoring disruption molecule is capable of associating with or binding to PKA RI and has been designed for and is intended for use in affecting the PKA type I signalling pathway. Preferably therefore the anchoring disruption molecule binding to PKA RI is specific in that the anchoring disruption molecule of the invention has a higher affinity for PKA RI than it does for RII and may thus be considered specific for RI. Preferably the binding affinity for RI is 50 times higher for RI than RII, even more preferably 100 times, 200 times, 800 times, 1000 times or 2000 times higher for RI than RII. This may be measured as described above. Preferably the affinity or specificity for RI is higher than that of known AKAP inhibitors such as PV38, e.g. more than 20, 50 or 100 fold higher affinity or specificity than PV 38 for RI.

This selectivity may also be expressed in terms of the amount of anchoring disruption molecule required to achieve the effect of inhibiting the PKA RI-AKAP interaction. For example in in vitro assays, selectivity is present (for inhibition of e.g. type I relative to type II) when at least a 5-fold lower concentration of said inhibitor is required to reduce binding between the RI and AKAP than RII and AKAP by 50%. Especially preferably at least a 10 or 100 fold lower concentration is required.

Conveniently said binding may be assessed according to the K_{D} between PKA RI and an AKAP in the presence of the anchoring disruption molecule. Said binding may alternatively be assessed according to the K_{D} between the anchoring disruption molecule and the binding site of the PKA RI molecule as discussed above. Preferably the K_{D} should be 1-500 nM, preferably 0.01-10 nM when assessed in vitro. This can be assessed by any appropriate techniques which measures binding between two binding partners.

For example the dissociation constants (K_{D}) may be measured directly by fluorescence polarization, as described in the Examples, or using other standard techniques which are known in the art.

“AKAPs” as described herein are considered to be A kinase anchoring proteins which bind to PKA I in its first 90 amino acids (as described previously) and which themselves bind to membrane bound components. Examples include AKAP1, AKAP149, ezrin, FSCIA, FSCIB, merlin and AKAP82 including site A and B. The tests described above may be performed using any appropriate AKAP (when performed in isolation) or rely on naturally present AKAPs, e.g. when cell based assays are used. Preferably when AKAPs are provided they are selected from an AKAP mentioned above, e.g. ezrin.

“AKAP mimics” as described herein refer to molecules of the invention which have at least one of the functions of a naturally occurring AKAP, e.g. bind to PKA I and/or also bind to one or more membrane bound components and/or modulate signalling through PKA I. Said mimic may exhibit said function to a higher or lesser extent than the AKAP which it mimics, e.g. may have higher binding affinity. AKAP mimics which bind to PKA I preferably do so with high affinity.

To act as mimics which allow the formation of a functional PKA signalling complex, said mimics preferably include a targeting sequence to facilitate anchoring at a specific site. This site may be a site used by naturally occurring AKAPs or a site not in use under normal circumstances. Such targeting sequences may target the bound molecules to the mitochondria, ER, centrosomes or other appropriate location. Synthetic or known target sequences may be used, e.g. targeting domains from D-AKAP1 (for targeting to the mitochondria and ER) or AKAP450 (for targeting to the centrosome) may be used.

These mimics thus may bind and act as AKAPs and allow the formation of relevant complexes to achieve PKA type I signalling. Molecules which are able to act as mimics may be determined using the same tests as described above to identify inhibitors, but the mimics that serve to activate the PKA type I signalling will show markers of enhanced rather than depressed PKA type I signalling.

Other mimics may not necessarily facilitate localization and complex formation and may instead bind to PKA type I and for example be used to identify or isolate the same, e.g. may bind and allow the identification or isolation of specific PKA type I isotypes. In such cases the mimic may be labelled. Other AKAP mimics may mimic a functional role of a naturally occurring AKAP by modulating signalling of the PKA type I pathway through means not necessarily involving a PKA I:AKAP interaction.

In a further feature therefore, the present invention thus provides a method of identifying and/or isolating a PKA type I molecule comprising contacting a sample containing said PKA molecule with an AKAP mimic as described herein, carrying a labelling means and capable of binding to PKA type I (e.g. PKA Iα) with high affinity and assessing the level of said AKAP mimic which is bound and/or isolating said PKA to which said AKAP mimic is bound, wherein said level of AKAP mimic is indicative of the level of said PKA molecule in said sample.

“Polypeptides” as referred to herein are molecules with preferably less than 100 amino acid residues but are preferably shorter, e.g. less than 50 amino acid residues in length, preferably 10 to 35, 14 to 30, or 14 to 25 amino acid residues in length. Sequence (1) consists of 18 residues to which one or more residues may be added at the C- and/or N-terminal end.

Polypeptides as described herein may be prepared by any conventional modes of synthesis, including chemical synthesis or recombinant DNA technology. Chemical synthesis may be performed by methods well known in the art involving cyclic sets of reactions of selective deprotection of the functional groups of a terminal amino acid and coupling of selectively protected amino acids, followed by complete deprotection of all functional groups. Synthesis may be performed in solution or on a solid support using suitable solid phases known in the art. Preferably the anchoring disruption molecules or AKAP mimics are substantially purified, e.g. pyrogen-free, e.g. more than 70%, especially preferably more than 90% pure (as assessed for example, in the case of peptides, by an appropriate technique such as peptide mapping, sequencing or chromatography). Purification may be performed for example by chromatography (e.g. HPLC, size-exclusion, ion-exchange, affinity, hydrophobic interaction, reverse-phase) or capillary electrophoresis.

As described above, peptidomimetics are also included within the scope of the invention. Peptidomimetics and analogues as referred to herein are molecules which mimic the peptide described above in terms of function (i.e. their ability to act as an anchoring disruption molecule or AKAP mimic as described herein using the tests described herein) and/or structure. Functionally said peptidomimetics and analogues may show some reduced efficacy in performing the anchoring disruption molecule or AKAP mimic function, but preferably are as efficient or are more efficient.

Peptides, particularly when used in biological, e.g. medical applications may not be without shortcoming as a result of e.g. poor oral and tissue absorption, rapid proteolysis cleavage, rapid excretion, potential antigenicity and poor shelf stability. One way in which this may be addressed is by the adoption of peptidomimetics which retain the functional features of the peptide but present them in the context of a different, e.g. non-peptide structure. Such peptidomimetics may have improved distribution, metabolism and pharmacokinetics profiles, e.g. improved stability and membrane permeability. Such peptidomimetics have successfully been developed and used for other particularly medical applications.

Peptidomimetics, particularly non-peptidic molecules may be generated through various processes, including conformational-based drug design, screening, focused library design and classical medicinal chemistry. Strategies that have been used to identify peptidomimetics from the parent peptide structure which serve as scaffolds for enhancing non-peptide character may include 3-dimensional conformation analysis of the peptide followed by the establishment of organic synthetic strategies to prepare non-peptidic analogues with similar or improved interaction with the pharmacophore groups on the ligand and the receptor. Thus for example various elements may be used to conformationally restrict certain relevant portions of the molecule, e.g. the distance between binding centers, α, β or γ turns, β-strands or α helices.

Thus not only may oligomers of unnatural amino acids or other organic building blocks be used, but also carbohydrates, heterocyclic or macrocyclic compounds or any organic molecule that comprises structural elements and conformation that provides a molecular electrostatic surface that mimics the same properties of the 3-dimensional conformation of the peptide may be used (Martin-Martinez et al., 2002, Bioorg. Med. Chem. Letters, 12, p 109-112; Andronati et al., 2004, Current Med. Chem., 11(9), p 1183-1211; Eguchi et al., 2003, Combinatorial Chemistry and High Throughput Screening, 6(7), p 611-621; Freidinger, 2003, J. Med. Chem., 46(26), p 5553-5566; Jones et al, 2003, Current Opin. Pharm., 3(5), p 530-543; Le et al., Drug Discovery Today, 8(15), p 701-709; Schirmeister & Kaeppler, 2003, Mini-reviews in Med. Chem., 3(4), 361-373; Eguchi & Kahn, Mini-reviews in Med. Chem., 2(5), p 447-462).

Thus the peptidomimetics may bear little or no resemblance to a peptide backbone. Peptidomimetics may comprise an entirely synthetic non-peptide form (e.g. based on a carbohydrate backbone with appropriate substituents) or may retain one or more elements of the peptide on which it is based, e.g. by derivatizing one or more amino acids or replacing one or more amino acids with alternative non-peptide components. Peptide-like templates include pseudopeptides and cyclic peptides. Structural elements considered redundant for the function of the peptide may be minimized to retain a scaffold function only or removed where appropriate. When peptidomimetics retain one or more peptide elements, i.e. more than one amino acid, such amino acids may be replaced with a non-standard or structural analogue thereof. Amino acids retained in the sequences may also be derivatised or modified (e.g. labelled, glycosylated or methylated) as long as the ability of the polypeptide to associate with or bind to PKA RI and compete with AKAP binding or act as an AKAP mimic is not compromised by the substitution, derivatisation or modification.

The peptidomimetics are referred to as being “derivable from” a certain polypeptide sequence. By this it is meant that the peptidomimetic is designed with reference to a defined polypeptide sequence, such that it retains the structural features of the peptide which are essential for its function. This may be the particular side chains of the polypeptide, or hydrogen bonding potential of the structure. Such features may be provided by non-peptide components or one or more of the amino acid residues or the bonds linking said amino acid residues of the polypeptide may be modified so as to improve certain functions of the polypeptide such as stability or protease resistance, while retaining the structural features of the polypeptide which are essential for its function. In other words the peptidomimetic or analogue has the same functional characteristics as a polypeptide having the defined sequence with respect to its ability to associate with or bind to PKA RI and to act as an anchoring disruption molecule or to act as an AKAP mimic and thereby alter the PKA RI signalling pathway. The peptidomimetic or analogue's functional characteristics are inherent from the structure of the peptidomimetic and the structure is designed to retain these properties. For example, in peptidomimetics retaining at least a partial amino acid content, one or more of these amino acid residues may be replaced with structural analogues, as long as the key structural features which provide the ability to bind to PKA RI are retained.

The ability of such a peptidomimetic to bind to PKA RI can readily be tested and it is possible to determine whether a peptidomimetic has the required function by performing routine binding assays such as those set out in the Examples.

It is preferred that if the peptidomimetic contains D-amino acids, these are found outside of the 18 residue consensus sequence, e.g. N- or C-terminal to the consensus sequence or in the targeting sequence. Preferred D amino acids are D-leucine and D-arginine.

Examples of non-standard or structural analogue amino acids which may be used are D amino acids, amide isosteres (such as N-methyl amide, retro-inverse amid, thioamide, thioester, phosphonate, ketomethylene, hydroxymethylene, fluorovinyl, (E)-vinyl, methyleneamino, methylenethio or alkane), L-N methylamino acids, D-α methylamino acids, D-N-methylamino acids. Examples of non-conventional amino acids are listed in Table 1.

TABLE 1

| Non-conventional amino acid | Code | Non-conventional amino acid | Code |
|---|---|---|---|
| α-aminobutyric acid | Abu | L-N-methylalanine | Nmala |
| α-amino-α-methylbutyrate | Mgabu | L-N-methylarginine | Nmarg |
| aminocyclopropane- | Cpro | L-N-methylasparagine | Nmasn |
| carboxylate | | L-N-methylaspartic acid | Nmasp |
| aminoisobutyric acid | Aib | L-N-methylcysteine | Nmcys |
| aminonorbornyl- | Norb | L-N-methylglutamine | Nmgln |
| carboxylate | | L-N-methylglutamic acid | Nmglu |
| cyclohexylalanine | Chexa | L-N-methylhistidine | Nmhis |
| cyclopentylalanine | Cpen | L-N-methylisolleucine | Nmile |
| D-alanine | Dal | L-N-methylleucine | Nmleu |
| D-arginine | Darg | L-N-methyllysine | Nmlys |
| D-aspartic acid | Dasp | L-N-methylmethionine | Nmmet |
| D-cysteine | Dcys | L-N-methylnorleucine | Nmnle |
| D-glutamine | Dgln | L-N-methylnorvaline | Nmnva |
| D-glutamic acid | Dglu | L-N-methylornithine | Nmorn |
| D-histidine | Dhis | L-N-methylphenylalanine | Nmphe |
| D-isoleucine | Dile | L-N-methylproline | Nmpro |
| D-leucine | Dleu | L-N-methylserine | Nmser |
| D-lysine | Dlys | L-N-methylthreonine | Nmthr |
| D-methionine | Dmet | L-N-methyltryptophan | Nmtrp |
| D-ornithine | Dorn | L-N-methyltyrosine | Nmtyr |
| D-phenylalanine | Dphe | L-N-methylvaline | Nmval |
| D-proline | Dpro | L-N-methylethylglycine | Nmetg |
| D-serine | Dser | L-N-methyl-t-butylglycine | Nmtbug |
| D-threonine | Dthr | L-norleucine | Nle |
| D-tryptophan | Dtrp | L-norvaline | Nva |
| D-tyrosine | Dtyr | α-methyl-aminoisobutyrate | Maib |
| D-valine | Dval | α-methyl-γ-aminobutyrate | Mgabu |
| D-α-methylalanine | Dmala | α-methylcyclohexylalanine | Mchexa |
| D-α-methylarginine | Dmarg | α-methylcylcopentylalanine | Mcpen |
| D-α-methylasparagine | Dmasn | α-methyl-α-napthylalanine | Manap |
| D-α-methylaspartate | Dmasp | α-methylpenicillamine | Mpen |
| D-α-methylcysteine | Dmcys | N-(4-aminobutyl)glycine | Nglu |
| D-α-methylglutamine | Dmgln | N-(2-aminoethyl)glycine | Naeg |
| D-α-methylhistidine | Dmhis | N-(3-aminopropyl)glycine | Norn |
| D-α-methylisoleucine | Dmile | N-amino-α-methylbutyrate | Nmaabu |
| D-α-methylleucine | Dmleu | α-napthylalanine | Anap |
| D-α-methyllysine | Dmlys | N-benzylglycine | Nphe |
| D-α-methylmethionine | Dmmet | N-(2-carbamylethyl)glycine | Ngln |
| D-α-methylornithine | Dmorn | N-(carbamylmethyl)glycine | Nasn |
| D-α-methylphenylalanine | Dmphe | N-(2-carboxyethyl)glycine | Nglu |
| D-α-methylproline | Dmpro | N-(carboxymethyl)glycine | Nasp |
| D-α-methylserine | Dmser | N-cyclobutylglycine | Ncbut |
| D-α-methylthreonine | Dmthr | N-cycloheptylglycine | Nchep |
| D-α-methyltryptophan | Dmtrp | N-cyclohexylglycine | Nchex |
| D-α-methyltyrosine | Dmty | N-cyclodecylglycine | Ncdec |
| D-α-methylvaline | Dmval | N-cylcododecylglycine | Ncdod |
| D-N-methylalanine | Dnmala | N-cyclooctylglycine | Ncoct |
| D-N-methylarginine | Dnmarg | N-cyclopropylglycine | Ncpro |
| D-N-methylasparagine | Dnmasn | N-cycloundecylglycine | Ncund |
| D-N-methylaspartate | Dnmasp | N-(2,2-diphenylethyl)glycine | Nbhm |
| D-N-methylcysteine | Dnmcys | N-(3,3-diphenylpropyl)glycine | Nbhe |
| D-N-methylglutamine | Dnmgln | N-(3-guanidinopropyl)glycine | Narg |
| D-N-methylglutamate | Dnmglu | N-(1-hydroxyethyl)glycine | Nthr |
| D-N-methylhistidine | Dnmhis | N-(hydroxyethyl))glycine | Nser |
| D-N-methylisoleucine | Dnmile | N-(imidazolylethyl))glycine | Nhis |
| D-N-methylleucine | Dnmleu | N-(3-indolylyethyl)glycine | Nhtrp |
| D-N-methyllysine | Dnmlys | N-methyl-γ-aminobutyrate | Nmgabu |
| N-methylcyclohexylalanine | Nmchexa | D-N-methylmethionine | Dnmmet |
| D-N-methylornithine | Dnmorn | N-methylcyclopentylalanine | Nmcpen |
| N-methylglycine | Nala | D-N-methylphenylalanine | Dnmphe |
| N-methylaminoisobutyrate | Nmaib | D-N-methylproline | Dnmpro |
| N-(1-methylpropyl)glycine | Nile | D-N-methylserine | Dnmser |
| N-(2-methylpropyl)glycine | Nleu | D-N-methylthreonine | Dnmthr |
| D-N-methyltryptophan | Dnmtrp | N-(1-methylethyl)glycine | Nval |
| D-N-methyltyrosine | Dnmtyr | N-methyla-napthylalanine | Nmanap |
| D-N-methylvaline | Dnmval | N-methylpenicillamine | Nmpen |
| γ-aminobutyric acid | Gabu | N-(p-hydroxyphenyl)glycine | Nhtyr |
| L-t-butylglycine | Tbug | N-(thiomethyl)glycine | Ncys |
| L-ethylglycine | Etg | penicillamine | Pen |
| L-homophenylalanine | Hphe | L-α-methylalanine | Mala |
| L-α-methylarginine | Marg | L-α-methylasparagine | Masn |
| L-α-methylaspartate | Masp | L-α-methyl-t-butylglycine | Mtbug |
| L-α-methylcysteine | Mcys | L-methylethylglycine | Metg |
| L-α-methylglutamine | Mgln | L-α-methylglutamate | Mglu |
| L-α-methylhistidine | Mhis | L-α-methylhomophenylalanine | Mhphe |
| L-α-methylisoleucine | Mile | N-(2-methylthioethyl)glycine | Nmet |
| L-α-methylleucine | Mleu | L-α-methyllysine | Mlys |
| L-α-methylmethionine | Mmet | L-α-methylnorleucine | Mnle |
| L-α-methylnorvaline | Mnva | L-α-methylornithine | Morn |
| L-α-methylphenylalanine | Mphe | L-α-methylproline | Mpro |
| L-α-methylserine | Mser | L-α-methylthreonine | Mthr |
| L-α-methyltryptophan | Mtrp | L-α-methyltyrosine | Mtyr |
| L-α-methylvaline | Mval | L-N-methylhomophenylalanine | Nmhphe |
| N-(N-(2,2-diphenylethyl) | Nnbhm | N-(N-(3,3-diphenylpropyl) | Nnbhe |
| carbamylmethyl)glycine | | carbamylmethyl)glycine | |
| 1-carboxy-1-(2,2-diphenyl- | Nmbc | L-O-methyl serine | Omser |
| ethylamino)cyclopropane | | L-O-methyl homoserine | Omhser |

Non-standard amino acids which may be used include conformationally restricted analogs, e.g. such as Tic (to replace F), Aib (to replace A) or pipecolic acid (to replace Pro).

Analogues also include molecules to which additional components have been added. This includes precursors of the anchoring disruption molecules or AKAP mimics (e.g. pro-drugs in which chemical modifications are made to the peptide that allow uptake from the intestine and also intracellular delivery and which are removed to release the active ingredient once administered to the body (e.g. by proteolytic cleavage)) or their peptidomimetics which may optionally be processed to yield the anchoring disruption molecule or AKAP mimic or peptidomimetic. Additional moieties may also be added to provide a required function, e.g. a moiety may be attached to assist or facilitate entry of the molecule into the cell. Analogues also include modestly truncated molecules as described hereinafter.

Peptidomimetics and analogues such as those exemplified above may be prepared by chemical synthesis or whether they retain amino acids, during synthesis of the polypeptide or by post production modification, using techniques which are well known in the art. Synthetic techniques for generating peptidomimetics from a known polypeptide are well known in the art.

An anchoring disruption molecule or AKAP mimic will therefore alter the PKA type I signalling pathway, when administered to a cell. The PKA type I signalling pathway may be up or down regulated, i.e. signalling may be increased or reduced.

The “PKA type I signalling pathway” as referred to herein refers to a series of signalling events in which PKA type I is activated (or not), resulting in increased (or reduced) kinase activity of this enzyme. This signalling pathway is intended to include molecular events from activation of PKA type I to end effects such as reduced proliferation or IL-2 production, or intermediate effects such as inactivation of Src kinases. Preferably the invention is concerned with PKA type Iα or PKA type Iβ, especially preferably PKA type Iα.

The “PKA type II signalling pathway” as referred to herein refers to a series of signalling events in which PKA type II is activated (or not), resulting in increased (or reduced) kinase activity of this enzyme. The end effects of PKA II signalling are as described herein.

As referred to herein the phrase “altering the activity of the PKA type I signalling pathway” is intended to mean the alteration of one or more signalling elements in the pathway (e.g. to affect its enzymatic or other functional properties) which affects downstream signalling events. “Alteration” of the signalling elements refers to the ability to form interactions with other molecules, e.g. protein-protein interactions. The ultimate effect is to down-regulate or up-regulate downstream events which typify PKA type I signalling. Alteration of said signalling pathway may be assessed by determining the extent of activation of a molecule involved in said pathway, e.g. phosphorylation of a kinase, e.g. Csk, or examination of levels of molecules whose levels are dependent on the activity of said pathway, e.g. IL-2. For use in particularly clinical conditions, down-regulation or up-regulation of the PKA type I signalling pathway, i.e. enhancing or reversing the effects of cAMP activation, e.g. to reverse lymphocyte dysfunction, is required depending on whether the clinical condition is typified by elevated or suppressed PKA type I signalling and/or would benefit from elevation or suppression of the same.

Preferably, the anchoring disruption molecule or AKAP mimic of the invention is a polypeptide of sequence 1 in which

X₁=L, X₂=E, X₃=Q, X₄=N, X₅=Q, X₆=D, X₇=Q, X₈=I, X₉=K, X₁₀=E, X₁₁=A, X₁₂=T and X₁₃=E,
i.e. comprises the following amino acid sequence:

### L E Q Y A N Q L A D Q I I K E A T E, (MEME3)

or a variant of said sequence wherein said variant has a substitution at any one, two, three, four or five of positions X₁ to X₁₃ of the sequence.

Preferably said one or more substitutions are selected from X₁=F, Y, I, V, W or C; X₂=C, D, R or K; X₃=F, K, R, A, I, L, M, S, T, V, G, N, W, D or E; X₄=K, R, G, T, S, W, D or E; X₅=S, F, K, R, M, W, Y, D or E; X₆=E, I, A, R, S, F, H, W, K, L, Y, M, N, Q or G; X₇=K, R, F, N, S, T, V, L, M, W, I, D or E; X₈=V; X₉=D, E, L, S, R, A, M, T, W, Y; X₁₀=D, R, K or Q; X₁₁=I or V; X₁₂=F, C, M, K, R, I or L; and X₁₃=D, N, V, Y, G, H, I, Q, A, F, K, L, M, R, S, T or W.

X₅ can also be L or T and X₁₁ can also be C.

Alternatively, said one or more substitutions are selected from X₁=C, I or F; X₂=D, K, R, V, A, I, L, Q, S or T; X₃=D, E, A, I, S or V; X₄=D, E, S, A, M or Q; X₅=D, E, C, I, M or F; X₆=G, S, E or M; X₇=D, E, I or M; X₈=V, D or E; X₉=A, D, E, M, R, T, W or Y; X₁₀=D; X₁₂=L, V or W; and X₁₃=D, R, K or W.

Especially preferably,

(i) single amino acid substitutions are selected from the group consisting of X₁=C; X₂=D; X₃=D or E; X₄=D or E; X₅=D or E; X₆=E, G or S; X₇=D, E or I; X₉=D, E, A, M, R, T, W or Y; X₁₀=D; and X₁₃=D (referred to as L1C, E2D, Q3D, Q3E, N6D, N6E, Q7D, Q7E, D10E, D10G, D10S, Q11D, Q11E, Q11I, K14D, K14E, K14A, K14M, K14R, K14T, K14W, K14Y, E15D and E18D respectively); further single amino acid substitutions are selected from X₁=F, I or Y; X₃=A, K, M, R, S or T; X₄=G or T; X₅=K, L M, R, S or T; X₆=N; X₇=K, L or R; X₁₂=C or M; X₁₃=M, N, Q or T (L1F, L1I, L1Y, Q3A, Q3K, Q3M, Q3R, Q11K, Q11L, Q11R, T17C, T17M, Q3S, Q3T, N6G, N6T, Q7K, Q7L, Q7M, Q7R, Q7S, Q7T, D10N, E18M, E18N, E18Q, E18T); and
(ii) double amino acid substitutions of said sequence are selected from the group consisting of:
a) when X₆ is S, either X₂ is K or D, X₅ is D or E or X₈ is V (alternatively referred to as D10S E2K, D10S E2D, D10S Q7D, D10S Q7E or D10S I12V);
b) when X₆ is E, either X₂ is K or D, X₅ is D or E or X₈ is V (alternatively referred to as D10E E2K, D10E E2D, D10E Q7D, D10E Q7E, D10E I12V); and
c) when X₈ is V, either X₂ is K or D, X₆ is M or X₁₂ is L (referred to as I12V E2K, I12V E2D, I12V D10M or I12V T17L);

alternatively, double amino acid sustitutions of said sequence may be selected from the group consisting of:

a) when X₃ is A, either X₄ is G; X₅ is K, M or R; X₇ is K, L or R; or X₁₃ is M, N or Q;
b) when X₁₃ is T, either X₁ is F; X₃ is A, K, E, M, R or S; X₄ is T or G; X₅ is M or R; X₇ is R or K; or X₉ is R;
c) when X₁₃ is Q, either X₃ is A, S, E, K, M, R or S; X₄ is G; X₅ is M, K or R; or X₇ is K; or
d) when X₁₃ is M, either X₃ is E, M, R, S or K; X₄ is G; X₅ is M, R or K; X₇ is R; or X₁₂ is C or M;
e) when X₇ is K, either X₃ is A, R, E, K or M; X₄ is G; X₅ is M; X₉ is R; X₁₂ is C or M; or X₁₃ is Q, T, M or N; and
f) when X₅ is M, either X₇ is K; or X₁₃ is N, Q or T;
(iii) triple amino acid substitutions of said sequence are selected from the group consisting of:
a) when X₂ is K, X₄ is E and X₆ is S (E2K N6E D10S);
b) when X₂ is D, X₄ is D or E and X₆ is E or S (E2D N6D/N6E D10E/D10S);
c) when X₂ is K, X₆ is M and X₈ is V (E2K D10M I12V);
d) when X₂ is D, X₆ is E or M and X₈ is D, E or V (E2D D10E/D10M I12D/I12E/I12V);
e) when X₇ is D, X₈ is V and X₁₂ is L (Q11D I12V T17L);
f) when X₇ is E, X₈ is V and X₁₂ is L (Q11E I12V T17L);
g) when X₆ is S, X₈ is V and either
X₁ is I or F;
X₂ is A, I, L, Q, S, T, D or V;
X₃ is E, D, S, A, I or V;
X₄ is S, E, D, A, M or Q;
X₅ is M, D, E, I or F;
X₇ is D, E, M;
X₉ is R;
X₁₀ is D;
X₁₂ is W or L; or
X₁₃ is K or W; and
   h) when X₆ is E, X₈ is V and either
X₁ is I or F;
   X₂ is A, I, L, Q, S, T, D or V;
   X₃ is E, D, S, A, I or V;
   X₄ is S, E, D, A, M or Q;
   X₅ is M, D, E, I or F;
   X₇ is D, E, M;
   X₉ is R;
   X₁₀ is D;
   X₁₂ is W or L; or
   X₁₃ is K or W; and
      iv) quadruple amino acid substitutions of said sequence are selected from the group consisting of:
      a) when X₅ is E, X₆ is S, X₈ is V and either
X₃ is A, D, E or S;
X₄ is E, D or S;
X₁₂ is L or W;
   b) when X₅ is D, X₆ is S, X₈ is V and either
X₃ is A, D, E or S;
X₄ is E, D or S;
X₁₂ is L or W;
   c) when X₄ is E, X₆ is S or E, X₈ is V and X₂ is either K, D, V, A, I, L, Q, S or T;
   d) when X₄ is D, X₆ is S or E, X₈ is V and X₂ is either K, D, V, A, I, L, Q, S or T;
   e) when X₂ is K, X₆ is S, X₈ is V and either
X₁ is F;
X₃ is E, D, A or S;
X₄ is S, A, M, D or E;
X₅ is F, I, M, D or E;
X₇ is M, D or E;
X₁₂ is L or W; or
X₁₃ is D or K;
   f) when X₂ is D, X₆ is S, X₈ is V and either

X₁ is F;
X₃ is E, D, A or S;
X₄ is S, A, M, D or E;
X₅ is F, I, M, D or E;
X₇ is M, D or E;
X₁₂ is L or W; or
X₁₃ is D or K;
   g) when X₂ is V; X₃ is E or D, X₆ is S or E and X₈ is V;
   h) when X₂ is D; X₃ is E or D, X₆ is S or E and X₈ is V;
   i) when X₂ is K; X₄ is E or D, X₆ is M or E and X₈ is V; and
   j) when X₂ is D; X₄ is E or D, X₆ is M or E and X₈ is V; and
   v) quintuple amino acid substitutions of said sequence are selected from the groups consisting of:
   a) when X₂ is K, X₄ is E or D, X₆ is S or E, X₈ is V and either
X₁ is I or F;
X₃ is D, E, A, I, S or V;
X₅ is M, F, D, E or I;
X₇ is D, E or M;
X₉ is R;
X₁₁ is D;
X₁₂ is L or W; or
X₁₃ is K, D or W;
   b) when X₂ is D, X₄ is E or D, X₆ is S or E, X₈ is V and either
X₁ is I or F;
X₃ is D, E, A, I, S or V;
X₅ is M, F, D, E or I;
X₇ is D, E or M;
X₉ is R;
X₁₀ is D;
X₁₂ is L or W; or
X₁₃ is K, D or W;
   c) when X₂ is A, X₄ is E or D, X₅ is C, D or E, X₆ is S or E and X₈ is V;
   d) when X₂ is D, X₄ is E or D, X₅ is C, D or E, X₆ is S or E and X₈ is V;
   e) when X₂ is T, X₄ is E or D, X₅ is E or D, X₆ is S or E and X₈ is V;
   f) when X₂ is Q, X₄ is E or D, X₅ is E or D, X₆ is S or E and X₈ is V; and
   or a peptidomimetic or analogue thereof.

Particularly preferred anchoring disruption molecules or AKAP mimics are polypeptides comprising the above sequence or the sequence with a single amino acid substitution particularly as described above.

Highly preferred are polypeptides comprising the above sequence with a single amino acid substitution selected from the group consisting of X₇=D or E or X₉=A, M, T, W or Y.

In a further preferred feature, the anchoring disruption molecule or AKAP mimic of the invention is a polypeptide of sequence 1 in which

X₁=L, X₂=K, X₃=Q, X₄=N, X₅=Q, X₆=S, X₇=Q, X₈=V, X₉=K, X₁₀=E, X₁₁=A, X₁₂=T and X₁₃=E,
i.e. comprising the following sequence:

### L K Q Y A N Q L A S Q V I K E A T E

or a variant thereof, wherein said variant has a substitution at any one, two, three or four of positions X₁ to X₁₃ of said sequence.

Preferably said one or more substitutions are selected from X₁=F or I; X₂=A, E, D, R, V, Q, S, I, L or T; X₃=A, S, E, D, V or I; X₄=A, M, S, E, D or Q, X₅=F, I, M, D or E; X₆=M, E or D; X₇=M, E or D; X₈=I; X₉=R or C; X₁₀=D; X₁₁=C; X₁₂=L, C or W; and X₁₃=D or the substitutions defined for the previous preferred sequence.

Alternatively, said one or more substitutions are selected from X₁=C, I or F; X₂=D, R, V, A, I, L, Q, S, E or T; X₃=D, E, A, I, S or V; X₄=D, E, S, A, M or Q; X₅=D, E, I, M or F; X₆=D, E, G or M; X₇=D, E, I or M; X₈=I; X₉=A, D, E, M, R, T, W or Y; X₁₀=D; X₁₂=L or W; and X₁₃=K, R, D or W.

Especially preferably

(i) single amino acid substitutions of the sequence are selected from the group consisting of X₂=A, D, E, V, Q, S, I, L or T; X₃=D, E, S or A; X₄=D, E, A, S or M; X₅=D, E or M; X₆=D or E; X₇=D or E; X₈=I; X₉=C; X₁₀=D; X₁₂=W or L; and X₁₃=D; and
(ii) double amino acid substitutions of said sequence are selected from the group consisting of:
(a) when X₂ is E or D, either
X₁ is I or F;
X₃ is A, E, D, S, I or V;
X₄ is A, E, D, S, M or Q;
X₅ is F, I, D, E or M;
X₆ is M, D or E;
X₇ is D, E or M;
X₈ is I;
X₉ is R;
X₁₀ is D;
X₁₂ is L or W; or
X₁₃ is D, K or W;
   (b) when X₆ is E or D, either
X₁ is I or F;
X₂ is A, D, E, S, I, V, L, Q or T;
X₃ is A, D, E, S, I or V;
X₄ is D or E;
X₅ is F, I, D, E or M;
X₆ is M, D or E;
X₇ is M, D or E;
X₈ is I;
X₉ is R;
X₁₀ is D;
X₁₂ is L or W; or
X₁₃ is W, K or D; and
   (c) when X₂ is V, X₃ is E or D; and
   (iii) triple amino acid substitutions of said sequence are selected from the group consisting of:
   (a) when X₂ is E or D and X₅ is E or D, either
X₃ is S, D, E or A;
X₄ is E, D or S;
X₈ is I; or
X₁₂ is W or L;
   (b) X₂ is T and X₄ and X₅ are both E or D;

### (c) X₂ is A, X₄ is E or D and X₅ is E or D;

(d) X₂ is E or D, X₆ is D or E and either X₈ is I or X₁₂ is L;

### (e) X₂ is E or D, X₆ is G and X₈ is I;

### (f) X₂ is V, X₄ is E or D and X₅ is E or D;

### (g) X₂ is D or E, X₆ is D or E and X₈ is I;

### (h) X₂ is Q, X₄ is E or D and X₅ is E or D; and

### (i) X₂ is E, X₆ is N and X₈ is I; and

(iv) quadruple amino acid substitutions of said sequence are selected from the group consisting of:
(a) when X₂ is E or D, X₆ is D or E and X, is I, either
X₁ is C;
X₃ is D or E;
X₄ is D or E;
X₅ is D or E;
X₇ is D, E or I; or
X₉ is A, D, E, M, R, T, W or Y;
X₁₂ can be C or M;
X₁₃ can be M, N, Q or T; and
X₁ can also be F, I or Y;
X₃ can also be A, K, M, R, S or T;
X₄ can also be G or T;
X₅ can also be K, L, M, R, S or T;
X₇ can also be K, L or R;
   (b) X₂ is E or D, X₆ and X₇ are both D or E and X₁₂ is L, or a peptidomimetic or analogue thereof.

Alternatively, the polypeptide may consist of any of the above sequences, or a 1-6, e.g. 1, 2, 3 or 4 (preferably 1 or 2) amino acid N- or C-terminal (preferably C-terminal) truncation of said polypeptide, provided that said truncated polypeptide retains the ability to bind to PKA I. Such polypeptides fall within the scope of analogues as described herein. Examples of truncated polypeptides which are particularly useful are:

LVQYAEQLASQVIKEAT
LESYANQLASQVIKEAT
LESYASQLASQVIKEAT
LEQYAEQLASQVIKEAT
LEQYAEQLASQVIKEA
LVQYAEELASQVIKEAT
LVQYAEELASQVIKEA
LESYANELASQVIKEAT
LESYANELASQVIKEA
LEQYASELASQVIKEAT
LEQYAEELASQVIKEAT
LEQYAEELASQVIKEA
LEQYANELASQIIKEAT
LEQYANELASQIIKEA
LEQYANELASQVIKEAL
LEQYANELASQVIKEA
LEQYANQLASQIIKEAT
LEQYANQLASQIIKEA
LEQYANQLASQVIKEAL
LEQYANQLASQVIKEA
LEQYANQLADQIIKEAT
LEQYANQLADQIIKEA
LEQYANQLADDVIKEAL
LEQYANQLADDVIKEA
LEQYANQLADDIIKEAT
LEQYANQLADDIIKEA
LEQYANQLADDVIKEAL
LEQYANQLADDVIKEA

In a preferred aspect the sequence:

### L K Q Y A N Q L A S Q V I K E A T E

is substituted with E (or D) at position 2 (i.e. X₂=E (or D) in sequence 1) and/or in combination with a second substitution such that

X₁ is I; or

X₃ is A, E, D or S; or

X₄ is A, E, D, S, M or Q; or

X₅ is F, D, E or M; or

X₆ is D or E; or

X₇ is M, D or E; or

X₈ is I; or

X₉ is R; or

X₁₀ is D; or

X₁₂ is L or W; or

X₁₃ is W or D.

Alternatively said polypeptide preferably has E (or D) at position 6 (i.e. X₄=E (or D) in sequence 1) and/or in combination with a second substitution such that

X₁ is I or F; or

X₂ is A, E, D, S, I, V, L, Q or T; or

X₃ is A, E, D, S, I or V; or

X₅ is F, D or E; or

X₆ is D or E; or

X₇ is D or E; or

X₈ is I; or

X₉ is R; or

X₁₀ is D; or

X₁₂ is L; or

X₁₃ is D.

In particularly preferred aspects of the invention, where present D may be replaced with E (or vice versa) and/or R may be replaced with K (or vice versa). In particularly preferred features X₂, X₃, X₄, X₅, X₆, X₇, X₁₀ and X₁₃ in the above described sequences are D or E and/or X₉ is K or R. Preferably molecules as described herein adopt the configuration of an amphipathic helix (though not necessarily by virtue of amino acids as non-peptide structural homologs may be used) and one face of said helix has at least 30% acidic amino acids (e.g. D or E) and at least 50% of said charged face has polar amino acids (e.g. G, S, C, Y, N or Q) wherein said 30 or 50% may also relate to the relative proportion contributed by non-peptide moieties.

In a preferred embodiment of the sequences described above, X₂ is E, X₉ is K, and X₁, X₃-X₈ and X₁₀-X₁₃ are as described above.

In an alternative embodiment X₄ is N and/or X₆ is D and/or X₁₀ is E and the remaining residues are as defined above.

In a different preferred embodiment, residue X₁ is L, X₈ is I and X and X₁₁ is A and the remaining residues are as defined above.

Alternatively X₂ is E, X₄ is N, X₆ is D, X₉ is K, X₁₀ is E and the remaining residues are as defined above.

In an alternative preferred aspect of the invention PKA I anchoring disruption molecules or AKAP mimics are provided in which the amino acid sequence 1 is modified and comprises the following amino acid sequence:

### X₁ X₂ X₃ W A X₄ X₅ L A X₆ X₇ X₈ I X₉ X₁₀ X₁₁ X₁₂ X₁₃ (2)

in which Y at the fourth position is substituted with W and X₁ to X₁₃ are as described hereinbefore.
Particularly preferably this substitution is made in the sequence LEQYANQLADQIIKEATE which has other preferred substitutions as described hereinbefore.

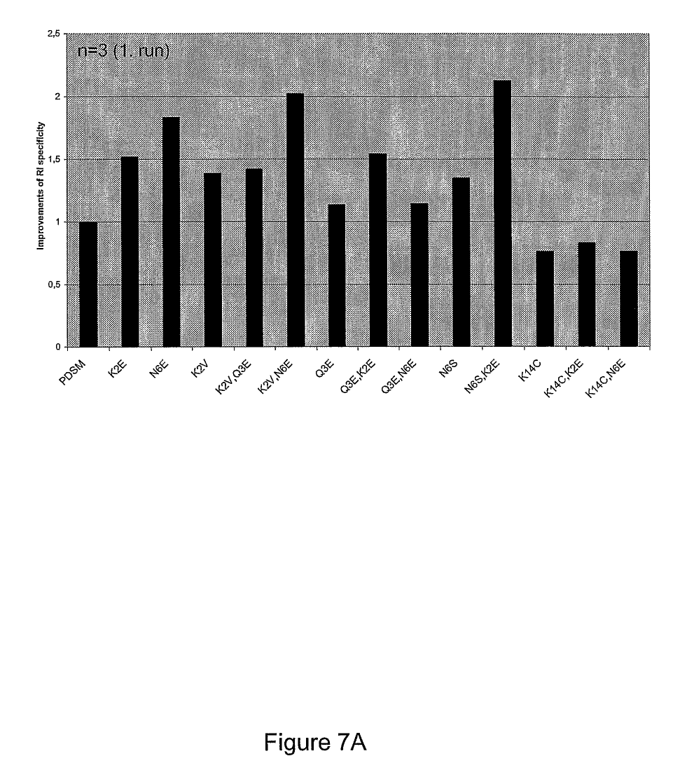

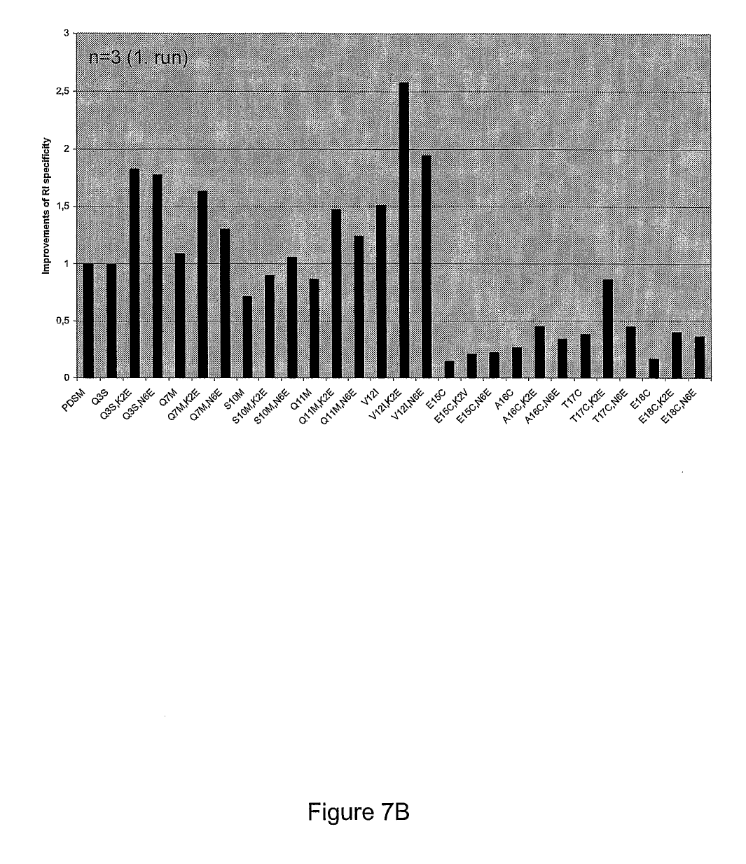

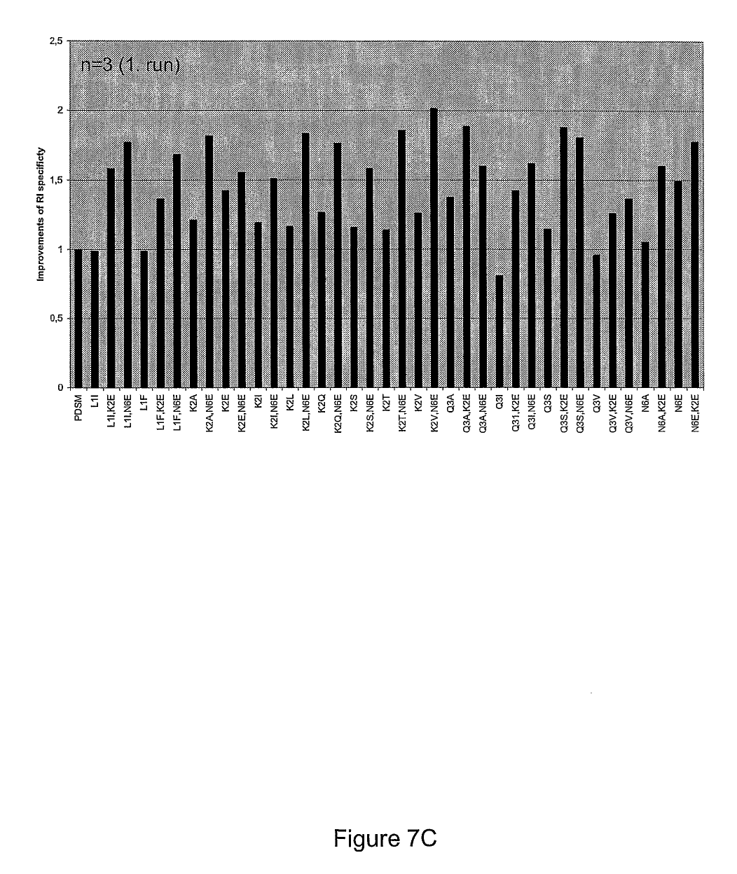

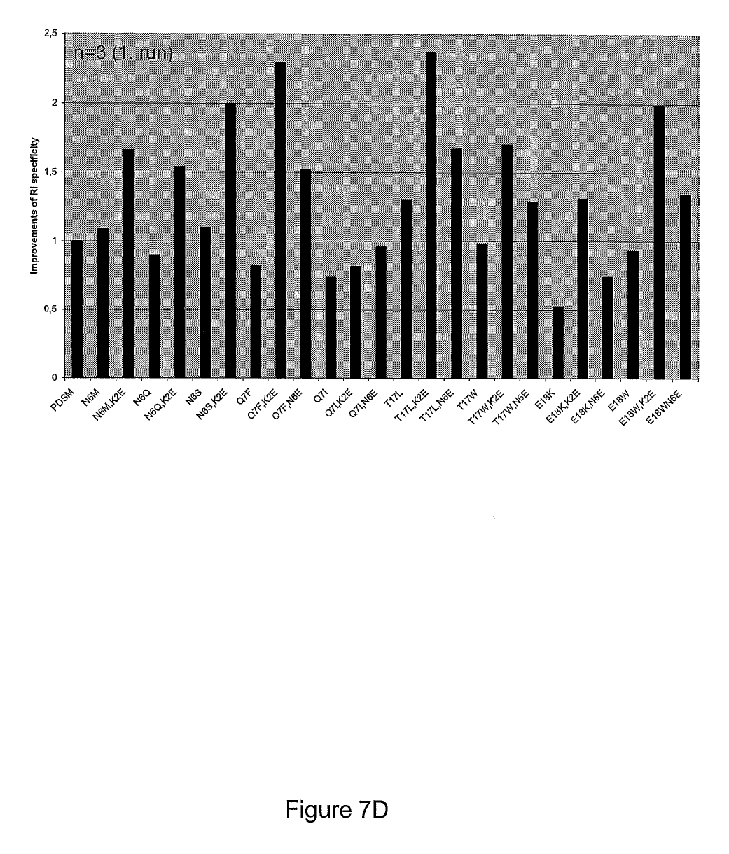

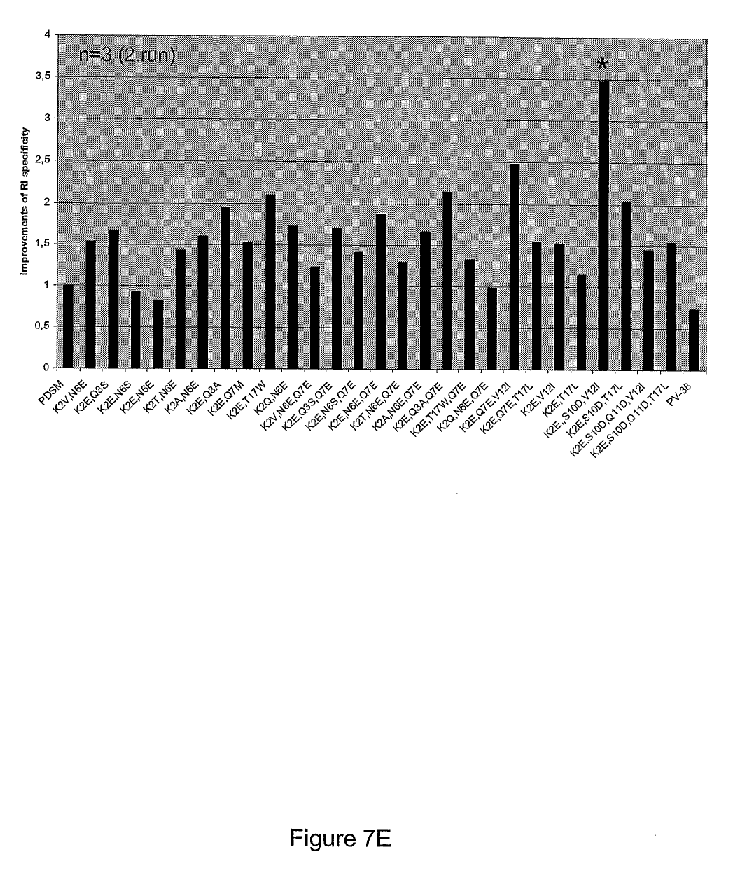

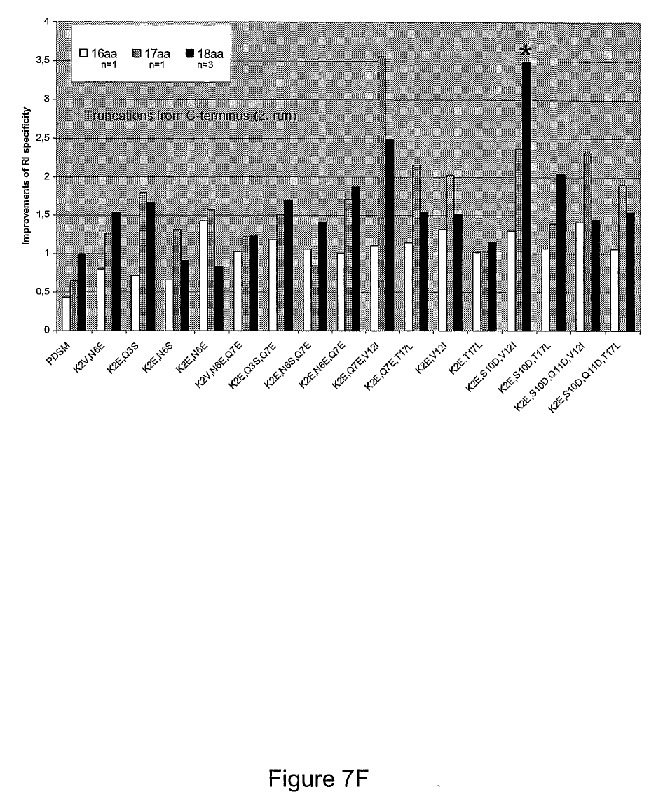

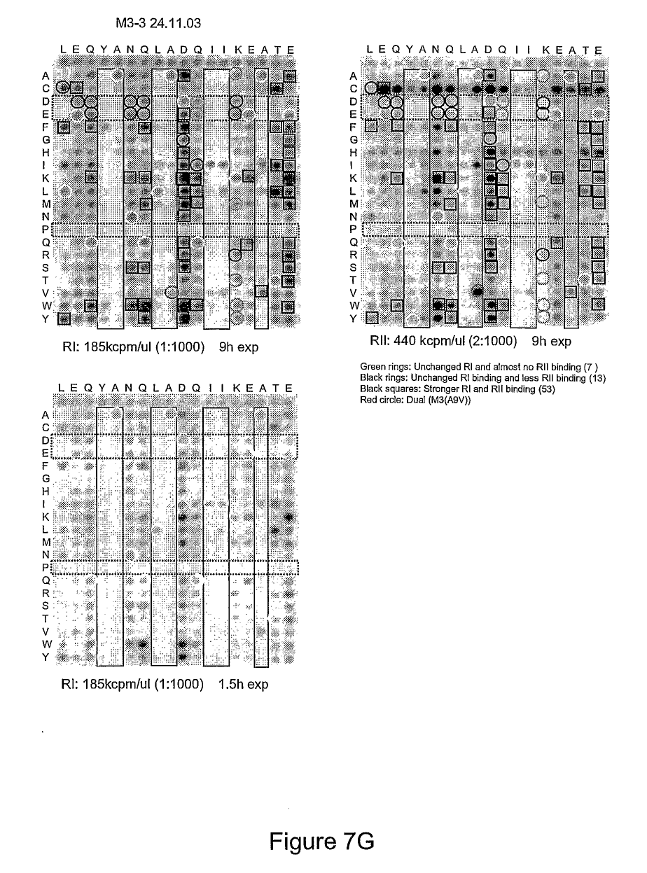

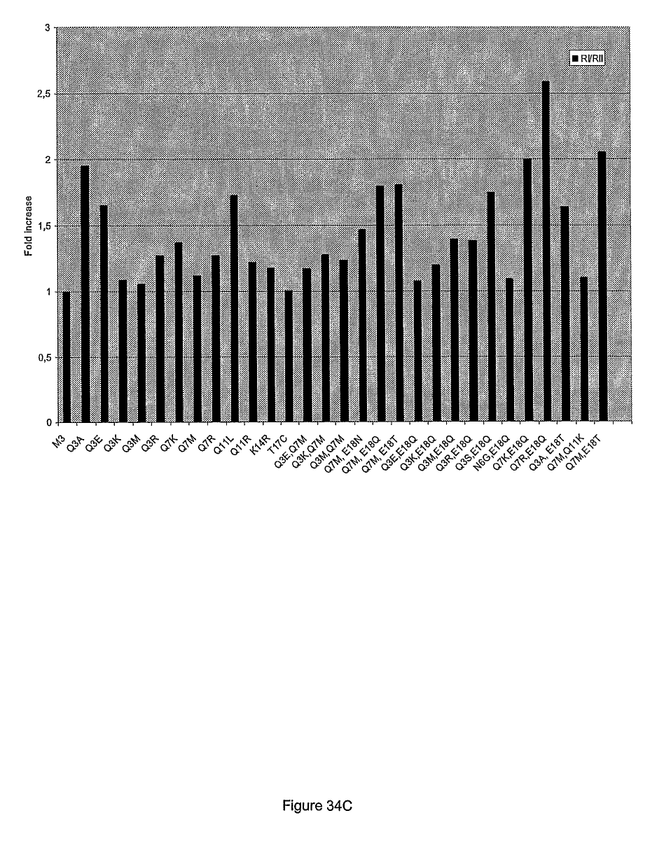

Specifically preferred polypeptides are as described in the Examples, for example those which achieve high improvements in specificity as disclosed in FIGS. 7 and 34.

Especially preferred peptides of the invention exhibit improved RI specificity relative to PDSM of more than 1-5, e.g. more than 2.0 or 3.0. Similarly, preferred peptides exhibit improved specificity relative to MEME3 of more than 2, 3, 4, 5, 10 or 20 fold.

In an alternative embodiment the anchoring disruption molecule or AKAP mimic of the invention further comprises an amino acid sequence which assists cellular penetration of said anchoring disruption molecule or AKAP mimic. Said additional amino acid sequence may for example be a polyarginine sequence, e.g. having from 3 to 16 residues, e.g. 8-12, preferably R₉, R₁₀ or R₁₁ or the HIV tat sequence or antennaepedia peptide (penetratin).

It will be noted from the Examples provided herein that certain peptides have particular utility as anchoring disruption molecules or AKAP mimics in respect of PKA II. The invention thus extends to such molecules for uses and methods as described herein in respect of the PKA II pathway. In particular such molecules have the sequence:
LKQYANQLASQVIKEATE
   in which E at position 15 is substituted with A, C, F, G, H, I, K, L, M, N, Q, R, S, T, V, W or Y and/or E at position 18 is substituted with A, C, F, G, H, K, L, M or R and/or A at position 9 is substituted with V. Other residues may be modified as described above in relation to the RI specific sequences as described hereinbefore. Such peptides are useful in treating disorders in which PKA II signalling is critical, such as metabolic disorders, asthma, chronic obstructive pulmonary disease, cardiovascular disease, neurological disorders, erectile dysfunction, diabetes insipidus, hypertension, gastric ulcers, thyroid disease, diabetes mellitus, post-infarction heart failure, weight loss (e.g. for treating obesity) or weight gain and male fertility. Affinity binding may be assessed relative to the binding of QIEYLAKQIVDNAIQQA of AKAP-IS (which is an exemplary RII AKAP) and PKA II binds AKAPs through its residues 1-50, particularly 1-44, more specifically 1-23. Markers of PKA II signalling include in the heart: increased heart rate, increased cardiac output, increased speed of Ca²⁺ release and reuptake (phosphorylation of b2-AR, L-type Ca²⁺ channel, RYR, phospholamban); in adipocytes: increased lipolysis (phosphorylation of hormone sensitive lipase and perilipin); in the kidney: water reabsorption due to aquaporin 2 phosphorylation and translocation to the apical membrane; in pancreatic β cells: insulin release through synapsin phosphorylation). Such markers may be examined in individuals, organs or cells as appropriate.

The present invention also extends to antibodies (monoclonal or polyclonal) and their antigen-binding fragments (e.g F(ab)₂, Fab and Fv fragments ie. fragments of the “variable” region of the antibody, which comprises the antigen binding site) directed to the anchoring disruption molecules or AKAP mimics as defined hereinbefore, ie. which bind to epitopes present on the anchoring disruption molecules and/or AKAP mimics and thus bind selectively and specifically to such anchoring disruption molecules and/or AKAP mimics relative to binding to other molecules such as AKAPs as described herein and which may be used to inhibit the binding of PKA RI to AKAPs or to bind to PKA RI.

The invention also relates to nucleic acid molecules comprising a sequence encoding a polypeptide described above.

The nucleic acid molecules described above may be operatively linked to an expression control sequence, or a recombinant DNA cloning vehicle or vector containing such a recombinant DNA molecule. This allows intracellular expression of the anchoring disruption molecule or AKAP mimic as a gene product, the expression of which is directed by the gene(s) introduced into cells of interest. Gene expression is directed from a promoter active in the cells of interest and may be inserted in any form of linear or circular DNA vector for incorporation in the genome or for independent replication or transient transfection/expression. Alternatively, the naked DNA molecule may be injected directly into the cell.

Appropriate expression vectors include appropriate control sequences such as for example translational (e.g. start and stop codons, ribosomal binding sites) and transcriptional control elements (e.g. promoter-operator regions, termination stop sequences) linked in matching reading frame with the nucleic acid molecules required for performance of the method of the invention as described hereinafter. Appropriate vectors may include plasmids and viruses (including both bacteriophage and eukaryotic viruses). Suitable viral vectors include baculovirus and also adenovirus, adeno-associated virus, herpes and vaccinia/pox viruses. Many other viral vectors are described in the art. Preferred vectors include bacterial and mammalian expression vectors pGEX-KG, pEF-neo and pEF-HA. The nucleic acid molecule may conveniently be fused with DNA encoding an additional polypeptide, e.g. glutathione-S-transferase, to produce a fusion protein on expression.

Thus viewed from a further aspect, the present invention provides a vector, preferably an expression vector, comprising a nucleic acid molecule as defined above.

Other aspects of the invention include methods for preparing recombinant nucleic acid molecules according to the invention, comprising inserting nucleotide sequences encoding the anchoring disurption molecule or AKAP mimic into vector nucleic acid.

In a further embodiment, the invention provides a method of altering the PKA type I signalling pathway in a cell by administration of an anchoring disruption molecule or AKAP mimic as defined herein or a molecule encoding such an anchoring disruption molecule or AKAP mimic.

In order to affect the signalling pathway, anchoring disruption molecules or AKAP mimics as described hereinbefore are conveniently added to a cell. This may be achieved by relying on spontaneous uptake of the anchoring disruption molecule or AKAP mimic into the cells or appropriate carrier means may be provided. Exogenous peptides or proteins may thus be introduced by any suitable technique known in the art such as in a liposome, niosome or nanoparticle or attached to a carrier or targeting molecule (see hereinafter). Thus for example, as discussed above, the anchoring disruption molecule or AKAP mimic may be tagged with a suitable sequence that allows the anchoring disruption molecule or AKAP mimic to cross the cell membrane. An example of such a tag is the HIV tat sequence or a stretch of e.g. 11 arginines.

It will be appreciated that the level of exogenous molecules introduced into a cell will need to be controlled to avoid adverse effects. The anchoring disruption molecule or AKAP mimic may be transported into the cell in the form of the polypeptide or in the form of a precursor, e.g. with an attached moiety to allow passage across the cell membrane (e.g. via endocytosis, pinocytosis or macro pinocytosis) or for cell targeting or in a form which is only activated on conversion, e.g. by proteolysis or transcription and translation. To control adverse effects, peptides may be tested by appropriate routine assays to determine their effects. Suitable techniques for this purpose are described in Examples 13 to 15. The skilled person would therefore be able to determine readily whether a peptide or peptidomimetic might be likely to be considered toxic to cells. Furthermore, it is apparent that the toxicity of any compound used at high concentrations can have toxic effects when used at those high concentrations and such assays could be used in order to determine an appropriate concentration range for administering a peptide or peptidomimetic according to the invention.

The anchoring disruption molecule or AKAP mimic may be administered to a cell by transfection of a cell with a nucleic acid molecule encoding the anchoring disruption molecule or AKAP mimic. As mentioned above, the present invention thus extends to nucleic acid molecules comprising a sequence which encodes the anchoring disruption molecule or AKAP mimic described herein and their use in methods described herein. Preferably said nucleic acid molecules are contained in a vector, e.g. an expression vector.

Nucleic acid molecules of the invention, preferably contained in a vector, may be introduced into a cell by any appropriate means. Suitable transformation or transfection techniques are well described in the literature. A variety of techniques are known and may be used to introduce such vectors into prokaryotic or eukaryotic cells for expression. Preferred host cells for this purpose include insect cell lines, eukaryotic cell lines or E. coli, such as strain BL21/DE3. The invention also extends to transformed or transfected prokaryotic or eukaryotic host cells containing a nucleic acid molecule, particularly a vector as defined above.

A further aspect of the invention provides a method of preparing an anchoring disruption molecule or AKAP mimic of the invention as hereinbefore defined, which comprises culturing a host cell containing a nucleic acid molecule as defined above, under conditions whereby said anchoring disruption molecule or AKAP mimic is expressed and recovering said molecule thus produced. The expressed anchoring disruption molecule or AKAP mimic product forms a further aspect of the invention.

The invention also extends to an anchoring disruption molecule or AKAP mimic encoded by a nucleic acid molecule as hereinbefore described. This may be produced by expression of a host cell as described above.

Cells containing anchoring disruption molecules or AKAP mimics of the invention, introduced directly or by expression of encoding nucleic acid material form further aspects of the invention.

Nucleic acid molecules which may be used according to the invention may be single or double stranded DNA, cDNA or RNA, preferably DNA and include degenerate sequences. Ideally however genomic DNA or cDNA is employed.

Anchoring disruption molecules or AKAP mimics as described herein may be used to alter PKA RI signalling. Thus in a further aspect, the present invention provides a method of altering the PKA type I signalling pathway in a cell by administration of an anchoring disruption molecule or AKAP mimic (or a nucleic acid molecule encoding said anchoring disruption molecule or AKAP mimic) as defined herein. This method may be used in vitro, for example in cell or organ culture, particularly for affecting PKA type I signalling pathways which have been activated (or not) or to reduce the extent of endogenous signalling or to stimulate PKA type I signalling.

The method may also be used ex viva, on animal parts or products, for example organs or collected blood, cells or tissues, particularly when it is contemplated that these will be reintroduced into the body from which they are derived. In particular, in samples in which abnormal levels of PKA type I signalling are occurring, levels may be normalized, e.g. by inhibiting (or activating) the activity of the PKA type I signalling pathway, as necessary. In such a method of treatment, the sample may be harvested from a patient and then returned to that patient.

In this context, a “sample” refers to any material obtained from a human or non-human animal, including tissues and body fluid. “Body fluids” in this case include in particular blood, spinal fluid and lymph and “tissues” include tissue obtained by surgery or other means. Such methods are particularly useful when the anchoring disruption molecule or AKAP mimic is to be introduced into the body by expression of an appropriate nucleic acid molecule. T cells for example, could be treated in this way. In such methods the methods of treatment of the invention as described hereinafter comprise the initial step of obtaining a sample from an individual, contacting cells from said sample with an anchoring disruption molecule or AKAP mimic (or a nucleic acid molecule encoding an anchoring disruption molecule or AKAP mimic) of the invention and administering said cells of said sample to the individual. The step of contacting refers to the use of any suitable technique which results in the presence of said anchoring disruption molecule or AKAP mimic in cells of the sample.

The method may also be used in vivo for the treatment or prevention of diseases in which abnormal PKA type I signalling occurs and this will be discussed in more detail below.

As described previously the methods of altering PKA type I signalling have utility in a variety of clinical indications in which abnormal PKA type I signalling is exhibited. Alternatively the signalling may be at normal levels but alleviation of disease progression or symptoms may be achieved by reducing or elevating the levels of PKA type I signalling.

Abnormal signalling may be elevated or reduced relative to a normal cell, sample or individual.

In particular since PKA type I is a key negative regulator of T cell function, diseases which exhibit lymphocyte dysfunction are particular targets for this treatment. Specifically, the anchoring disruption molecules which abolish the function of PKA type I may be used to produce pharmaceutical preparations to treat immunosuppressive disease. AKAP mimics may be used to treat immune activation diseases, e.g. auto-immune disorders.

Thus, the anchoring disruption molecules or AKAP mimics may be used to treat or prevent disorders typified by aberrant PKA type I signalling or disorders or diseases in which PKA type I signalling has been implicated or disorders or diseases which would be alleviated (e.g. by a reduction in symptoms) by reducing or elevating PKA type I signalling. Such disorders include immunosuppressive disorders (such as HIV infection, AIDS or common variable immunodeficiency) or proliferative diseases in which PKA type I signalling has been implicated, e.g. cancers such as colorectal carcinoma, pancreatic carcinoma, hepatocellular carcinoma, cancer mamma, ovarian cancer, non-small cell carcinoma of the lung, leukemia, adenoma of the pituitary or thyroid, thyroid carcinoma and autoimmune diseases).

The invention further relates to an anchoring disruption molecule or AKAP mimic or their encoding nucleic acid molecule as defined herein for use in medicine. Preferably the diseases to be treated are diseases which exhibit lymphocyte dysfunction. Specifically, the anchoring disruption molecules or AKAP mimics which affect the function of PKA type I may be used to produce pharmaceutical preparations.

The invention further relates to the use of an anchoring disruption molecule or AKAP mimic as defined herein in the manufacture of a medicament for treating diseases with abnormal PKA type I signalling or disorders or diseases in which PKA type I signalling has been implicated or disorders or diseases which would be alleviated (e.g. by a reduction in symptoms) by reducing or elevating PKA type I signalling, e.g. which exhibit lymphocyte dysfunction, such as immunosuppressive diseases, such as HIV infection, AIDS or common variable immunodeficiency) or proliferative diseases in which PKA type I signalling has been implicated, e.g. cancers such as colorectal carcinoma, pancreatic carcinoma, hepatocellular carcinoma, cancer mamma, ovarian cancer, non-small cell carcinoma of the lung, leukemia, adenoma of the pituitary or thyroid, thyroid carcinoma and autoimmune diseases).

The invention also relates to a method of treating such diseases comprising the step of administering an affective amount of an anchoring disruption molecule or AKAP mimic as defined herein to a mammal in need thereof.

Preferred mammals are humans.

The anchoring disruption molecules or AKAP mimics as described herein may therefore be formulated as pharmaceutical compositions in which the anchoring disruption molecule or AKAP mimic may be provided as a pharmaceutically acceptable salt. Pharmaceutically acceptable salts may be readily prepared using counterions and techniques well known in the art.

The invention thus further extends to pharmaceutical compositions comprising one or more anchoring disruption molecules or AKAP mimics (e.g. nucleic acid molecules, or polypeptides as defined above) and one or more pharmaceutically acceptable excipients and/or diluents. By “pharmaceutically acceptable” is meant that the ingredient must be compatible with other ingredients in the composition as well as physiologically acceptable to the recipient.

The active ingredient for administration may be appropriately modified for use in a pharmaceutical composition. For example when peptides are used these may be stabilized against proteolytic degradation by the use of derivatives such as peptidomimetics as described hereinbefore. The active ingredient may also be stabilized for example by the use of appropriate additives such as salts or non-electrolytes, acetate, SDS, EDTA, citrate or acetate buffers, mannitol, glycine, HSA or polysorbate.

Conjugates may be formulated to provide improved lipophilicity, increase cellular transport, increase solubility or allow targeting. Conjugates may be made terminally or on side portion of the molecules, e.g. on side chains of amino acids. These conjugates may be cleavable such that the conjugate behaves as a pro-drug. Stability may also be conferred by use of appropriate metal complexes, e.g. with Zn, Ca or Fe.

The active ingredient may be formulated in an appropriate vehicle for delivery or for targeting particular cells, organs or tissues. Thus the pharmaceutical compositions may take the form of microemulsions, liposomes, niosomes or nanoparticles with which the active ingredient may be absorbed, adsorbed, incorporated or bound. This can effectively convert the product to an insoluble form. These particulate forms have utility for transfer of nucleic acid molecules and/or protein/peptides and may overcome both stability (e.g. enzymatic degradation) and delivery problems.

These particles may carry appropriate surface molecules to improve circulation time (e.g. serum components, surfactants, polyoxamine908, PEG etc.) or moieties for site-specific targeting, such as ligands to particular cell borne receptors. Appropriate techniques for drug delivery and for targeting are well known in the art and are described in WO99/62315. For an example of specific site directed targeting, see for example Schafer et al., 1992, Pharm. Res., 9, p 541-546 in which nanoparticles can be accumulated in HIV-infected macrophages. Clearly such methods have particular applications in the methods of the invention described herein.

Such derivatized or conjugated active ingredients are intended to fall within the definition of anchoring disruption molecules or AKAP mimics which form aspects of this invention.

Pharmaceutical compositions for use according to the invention may be formulated in conventional manner using readily available ingredients. Thus, the active ingredient may be incorporated, optionally together with other active substances as a combined preparation, with one or more conventional carriers, diluents and/or excipients, to produce conventional galenic preparations such as tablets, pills, powders, lozenges, sachets, cachets, elixirs, suspensions (as injection or infusion fluids), emulsions, solutions, syrups, aerosols (as a solid or in a liquid medium), ointments, soft and hard gelatin capsules, suppositories, sterile injectable solutions, sterile packaged powders, and the like. Biodegradable polymers (such as polyesters, polyanhydrides, polylactic acid, or polyglycolic acid) may also be used for solid implants. The compositions may be stabilized by use of freeze-drying, undercooling or Permazyme.

Suitable excipients, carriers or diluents are lactose, dextrose, sucrose, sorbitol, mannitol, starches, gum acacia, calcium phosphate, calcium carbonate, calcium lactose, corn starch, aglinates, tragacanth, gelatin, calcium silicate, microcrystalline cellulose, polyvinylpyrrolidone, cellulose, water syrup, water, water/ethanol, water/glycol, water/polyethylene, glycol, propylene glycol, methyl cellulose, methylhydroxybenzoates, propyl hydroxybenzoates, talc, magnesium stearate, mineral oil or fatty substances such as hard fat or suitable mixtures thereof. Agents for obtaining sustained release formulations, such as carboxypolymethylene, carboxymethyl cellulose, cellulose acetate phthalate, or polyvinylacetate may also be used.

The compositions may additionally include lubricating agents, wetting agents, viscosity increasing agents, colouring agents, granulating agents, disintegrating agents, binding agents, osmotic active agents, emulsifying agents, suspending agents, preserving agents, sweetening agents, flavouring agents, adsorption enhancers, e.g. for nasal delivery (bile salts, lecithins, surfactants, fatty acids, chelators) and the like. The compositions of the invention may be formulated to provide quick, sustained or delayed release of the active ingredient after administration to the patient by using procedures well known in the art.

The active ingredient in such compositions may comprise from about 0.01% to about 99% by weight of the formulation, preferably from about 0.1 to about 50%, for example 10%.

The invention also extends to pharmaceutical compositions as described above for use as a medicament.

In methods of the invention, anchoring disruption molecules or AKAP mimics should be used at appropriate concentrations such that a significant number of the relevant binding partners' interactions, are prevented or where mimics are used, such that PKA I signaling is increased relative to untreated samples or individuals.

Preferably the pharmaceutical composition is formulated in a unit dosage form, e.g. with each dosage containing from about 0.1 to 500 mg of the active ingredient. The precise dosage of the active compound to be administered and the length of the course of treatment will of course, depend on a number of factors including for example, the age and weight of the patient, the specific condition requiring treatment and its severity, and the route of administration. Generally however, an effective dose may lie in the range of from about 0.01 mg/kg to 20 mg/kg, depending on the animal to be treated, and the substance being administered, taken as a single dose.

For methods in which the anchoring disruption molecule or AKAP mimic or their encoding molecule is administered to a sample ex vivo to be returned to the body, suitable dosages of said anchoring disruption molecule are 25-100 nM or lower, such as 10-50 nM, 5-25 nM, 1-5 nM or 0.2-5 nM.

The administration may be by any suitable method known in the medicinal arts, including for example oral, parenteral (e.g. intramuscular, subcutaneous, intraperitoneal or intravenous) percutaneous, buccal, rectal or topical administration or administration by inhalation. The preferred administration forms will be administered orally, rectally or by injection or infusion. As will be appreciated oral administration has its limitations if the active ingredient is digestible. To overcome such problems, ingredients may be stabilized as mentioned previously and see also the review by Bernkop-Schnürch, 1998, J. Controlled Release, 52, p 1-16.

It will be appreciated that since the active ingredient for performance of the invention takes a variety of forms, e.g. nucleic acid molecule (which may be in a vector) or peptide, the form of the composition and route of delivery will vary. Preferably however liquid solutions or suspensions would be employed, particularly e.g. for nasal delivery and administration will be systemic.

As mentioned above, these pharmaceutical compositions may be used for treating or preventing conditions in which the PKA type I signalling pathway is abnormal, in particular when the activity of this pathway is elevated or reduced.

Thus, viewed from a further aspect the present invention provides a method of treating or preventing disorders exhibiting abnormal PKA type I signalling activity or which would benefit from a reduction or elevation in the levels of PKA type I signalling, preferably immunosuppressive disorders or proliferative diseases, in a human or non-human animal wherein a pharmaceutical composition as described hereinbefore is administered to said animal. Alternatively stated, the present invention provides the use of a pharmaceutical composition as defined above for the preparation of a medicament for the treatment or prevention of diseases or disorders exhibiting abnormal PKA type I signalling activity or which would benefit from a reduction or elevation in the levels of PKA type I signalling, preferably immunosuppressive disorders, proliferative diseases or autoimmune diseases.

As referred to herein a “disorder” or “disease” refers to an underlying pathological disturbance in a symptomatic or asymptomatic organism relative to a normal organism, which may result, for example, from infection or an acquired or congenital genetic imperfection. A “condition” refers to a state of the mind or body of an organism which has not occurred through disease, e.g. the presence of a moiety in the body such as a toxin, drug or pollutant.

As referred to herein an “immunosuppressive disorder” refers to a disorder which is typified by impaired function of cells involved in normal immune responses, particularly B and T cells, and is also referred to herein as immunodeficiency or immune dysfunction. Preferably virally-induced immunodeficiency disorders are treated. Preferred conditions for treatment according to the invention include infection by retroviruses, particularly HIV (and infection by related viruses in other animals, e.g. SIV, FIV, MAIDS) and the resultant AIDS and treatment of common variable immunodeficiency and related conditions to the aforementioned conditions.

Especially preferably the methods described herein may be used to reverse cAMP hyperactivation that produces T cell dysfunction in immunodeficiencies by interrupting cAMP and PKA-mediated signal transduction in T cells, particularly T cell lipid rafts.

As referred to herein “proliferative diseases” refers to those diseases in which aberrant proliferation of cells occurs, e.g. cancer. Preferably said cells are cells involved in the generation or maintenance of immune responses, particularly B or T cells. Such diseases concern decreases in proliferation relative to normal levels.

Subjects which may be treated are preferably mammalian, preferably humans and companion or agricultural animals such as dogs, cats, monkeys, horses, sheep, goats, cows, rabbits, rats and mice.

As used herein, “treating” refers to the reduction, alleviation or elimination, preferably to normal levels, of one or more of the symptoms of said disease, disorder or condition which is being treated, e.g. infectivity or a reduction or alleviation of immune dysfunction, relative to the symptoms prior to treatment. Where not explicitly stated, treatment encompasses prevention. “Preventing” refers to absolute prevention, i.e. absence of detectable infectious agents, e.g. virus and/or maintenance of normal levels with reference to the extent or appearance of a particular symptom (e.g. T cell numbers) or reduction or alleviation of the extent or timing (e.g. delaying) of the onset of that symptom.

The method of treatment according to the invention may advantageously be combined with administration of one or more active ingredients which are effective in treating the disorder or disease to be treated. Preferably such additional active ingredients are cAMP antagonists e.g. a thiosubstituted cAMP analogue (such as derivatives of adenosine-3′,5′-cyclic monophosphorothioate, Rp isomer, such as Rp-8-Br-cAMPS or Rp-8-Cl-cAMPS), or COX-2 inhibitors as described in WO02/07721, which is incorporated herein by reference.

Other examples, e.g. in the treatment of HIV or AIDS preferably the anchoring disruption molecule is used in combination with one or more NNRTIs (non-nucleoside reverse transcriptase inhibitors) or in combination with one or more NRTIs (nucleoside reverse transcriptase inhibitors) or in combination with one or more HIV protease inhibitors or one or more HAART (highly active antiretroviral therapy) in combination with the anchoring disruption molecule of this invention. Alternatively the composition of the invention may contain agents used in vaccination protocols for treating HIV or cancer, i.e. HIV or cancer vaccines. Thus, pharmaceutical compositions of the invention may additionally contain one or more of such active ingredients.

In a further aspect, the present invention provides methods and/or compositions which combine one or more anchoring disruption molecules or AKAP mimics as described herein with compounds that improve the tolerability of the active ingredient, especially during long term treatment. Typical compounds include antihistamine and proton pump inhibitors.

According to a yet further aspect of the invention we provide products containing one or more anchoring disruption molecules or AKAP mimics as herein defined and one or more additional active ingredients as a combined preparation for simultaneous, separate or sequential use in human or animal therapy.

The following Examples are given by way of illustration only in which the Figures referred to are as follows:

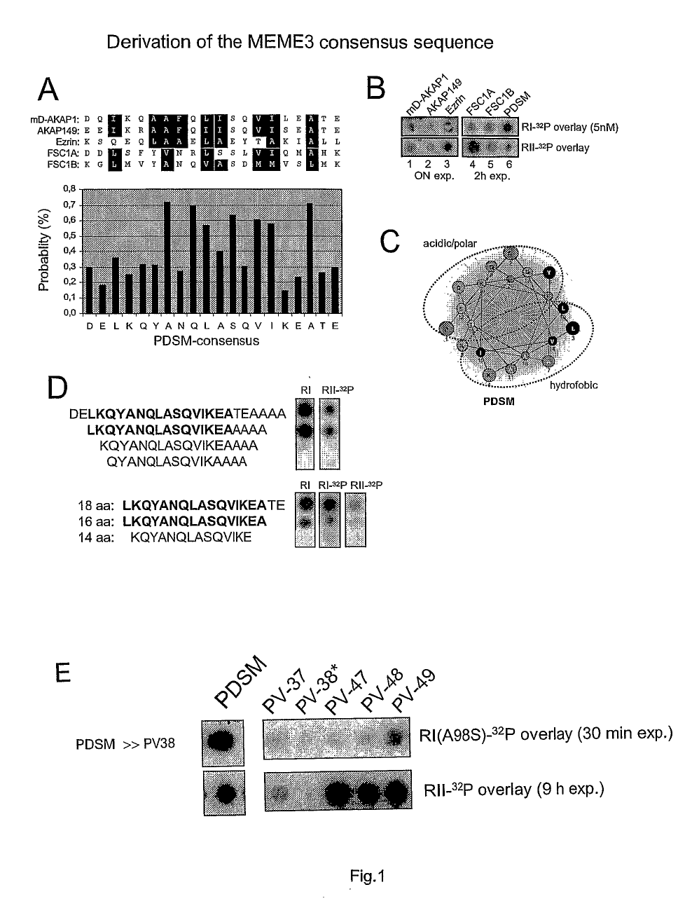

FIG. 1 shows the development of the PDSM consensus sequence

(A) alignment of sequences of the amphipathic helix motifs from mouse D-AKAP1, human AKAP149, human ezrin, human FSCIA and human FSCIB. Black panels indicate hydrophobic amino acids.
(B) Autoradiography of overlay/far western using radiolabelled RI (RI mutated (A98S) protein), upper panel or RII (lower panel) onto spotted 20 mer peptides corresponding to the amphipathic helix motifs of the indicated peptides.
(C) Helical wheel model prediction of the PDSM consensus sequence in an α-helical configuration.
(D) Autoradiography/immunoblotting of truncated versions of the PDSM sequences, with (upper panel) or without (lower panel) or polyalanine to radiolabelled RI or RII tail.
(E) Autoradiography showing PV peptides binding to R1 (A98S) PV-38 (denoted with a star) is the previously described peptide that binds RIα most specifically. The R-binding was analyzed by a solid-phase binding assay using ³²P-radiolabeled RIα.

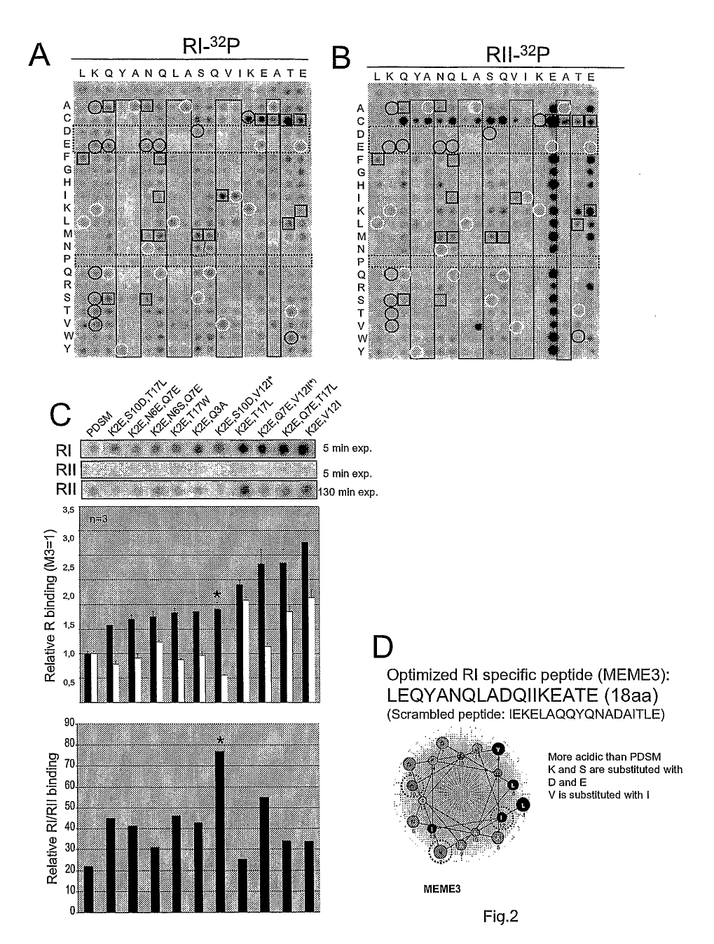

FIG. 2 shows optimisation of the PDSM 18 amino acid consensus sequence.

(A) RI³²P overlay on a 2 dimensional array of 360 PDSM derivatives in which each residue in the PDSM sequence (given by their single-letter codes above each array) was replaced with residues having every possible side chain (given by their single-letter codes to the left of each array). The first row in each array corresponds to the native peptide (PDSM). PDSM derivatives with higher RI affinity and lower RII affinity or higher affinity for both RI and RII are indicated by black circles and squares, respectively. White circles denote peptides in the array that correspond to the native PDSM sequence. (B) RII³²P overlay on the same 2 dimensional array as (A).
(C) Relative RI and RII binding affinities measured by densitometry analysis of the autoradiographs (n=3) of 10 PDSM derivatives with particularly high RI affinity, upper panel showing the array, and the lower 2 panels showing graphical representations. The middle panel depicts the levels of RI and RII displayed in the first panel and the bottom panel shows the RI/RII signal relative to the unaltered PDSM sequence. Two different exposures are shown for the RII experiment.
(D) Modelling of the MEME3 sequence in an α helical structure using the helical wheel model. The dotted circles represent substitutions relative to PDSM.

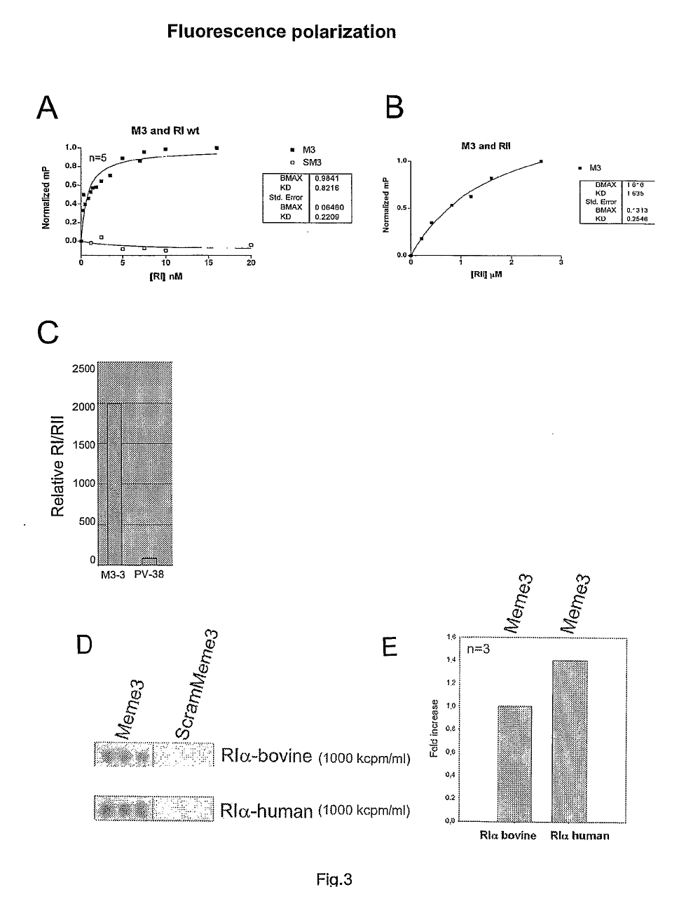

FIG. 3 shows dissociation constants (K_{D}) of MEME3 binding for RI and RII measured by fluorescence polarization. Dissociation constants (K_{D}) for MEME3 and scrambled sequence SM3 and (A) RI (bovine) or (B) RII (mouse) were measured by fluorescence polarization. MEME3 and PV-38 selectivity for RI over RII is shown (C).

Human RI binding to MEME3 is also shown in 3D & E.

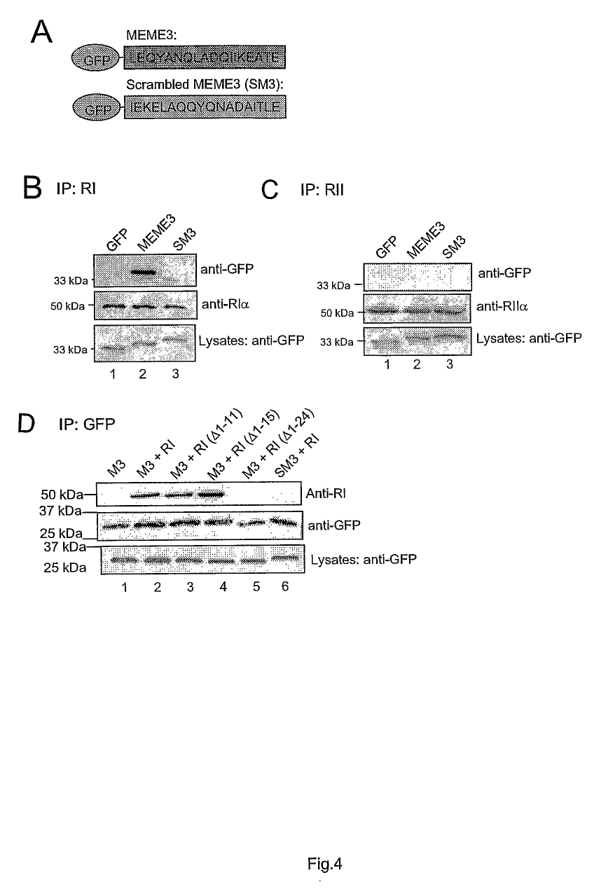

FIG. 4 shows specific interaction of MEME3 with RI and RII in vivo in cells.

(A) Schematic representation of the GFP-fusion proteins with the MEME3 (upper) and scrambled MEME3 (lower) peptides used in the cell culture studies.
(B-C) shows results of immunoprecipitation experiments on HEK293 cells transfected with either the MEME3, SM3 or GFP constructs, with RIα(B) or RII(C) antibodies, followed by SDS PAGE and Western blotting using the antibodies indicated. MEME3 was co-precipitated with RIα but not with RII.
(D) shows results of immunoprecipitation experiments on HEK293 cells transfected with MEME3, or SM3 and full length or truncated RI added to the lysate as indicated. Immunoprecipitation was performed using a monoclonal antibody against GFP and the preparation analysed by Western blotting with the antibodies shown. MEME3 was coprecipitated with full length RI, RI with the deletion of amino acids 1 to 11 and RI with deletion of amino acids 1 to 15, but not with RI with amino acids 1 to 24 deleted. Scrambled MEME3 was not coprecipitated with RI.

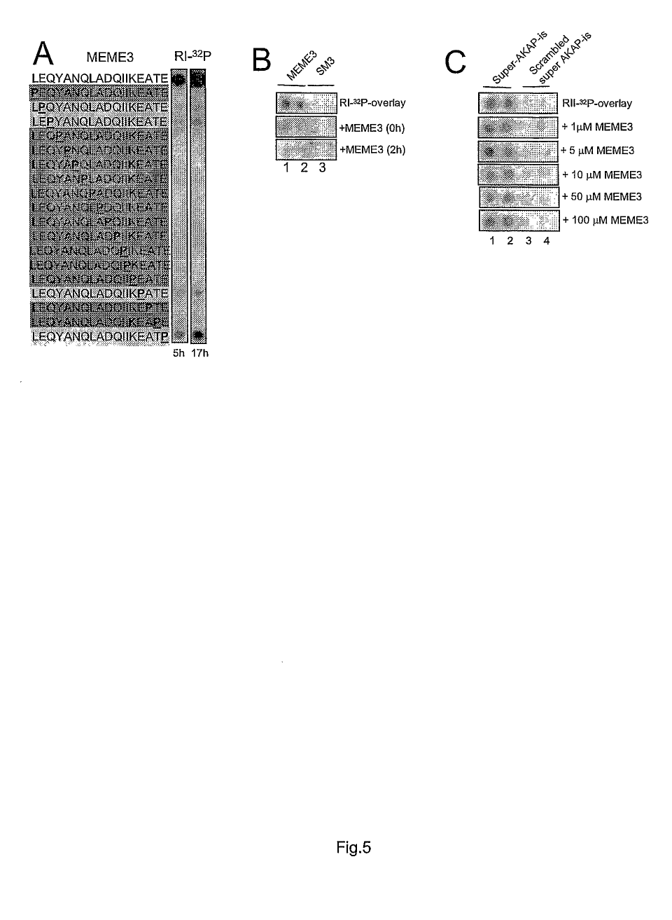

FIG. 5 shows in vitro characterisation of the MEME3 peptide.

(A) Results of RI ³²P overlay experiments in native MEME3 and proline substituted MEME3 synthesized on a membrane are shown.
(B&C) results of RI ³²P and RII ³²P overlay competition experiments.
500 nm MEME3 was added in solution and shown to compete membrane bound MEME3 binding to solution RI ³²P (B). Concentrations of up to 100 μM MEME3 were unable to compete the RII binding in solution to an RII specific binding sequence.

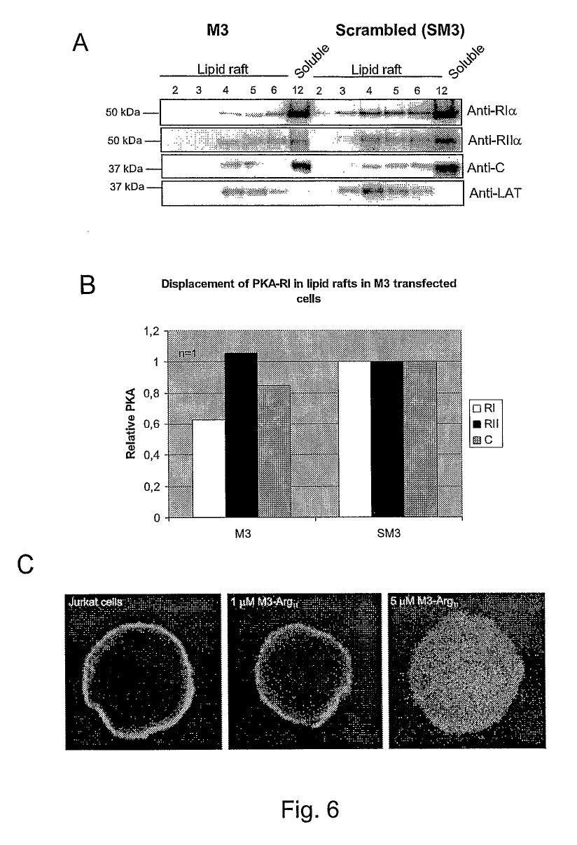

FIG. 6 shows displacement of PKA type I in lipid rafts in Jurkat T cells.

(A) Western blot on protein extracts from lipid rafts of Jurkat T cells transfected with MEME3 and scrambled MEME3 (SM3) using the antibodies indicated. Lane numbers depict fraction numbers from sucrose density gradient centrifugation and fractionation of lipid rafts (fractions 2-6) versus bottom fractions with other cellular membranes and cytoskeletal elements (fraction no. 12).
(B) Results of densitometry analysis of the results in (A). The concentration of RI is reduced in MEME3 transfected cells.
(C) Immunofluorescence of Jurkat T cells incubated with MEME3-Arg₁₁ at different concentrations.

FIG. 7 (A-E) show the relative RI specificity of several peptides in comparison to the PDSM peptide.

(F) shows the relative RI specificity of various N terminal truncated peptides.
(G) shows RI and RII overlay experiments on a 2 dimensional array of 360 MEME3 derivatives in which each having a higher RI specificity than MEME3, in which circles and squares are as described in FIG. 1. Arrows signify substitutions which result in unchanged association with RI but almost no RII binding.

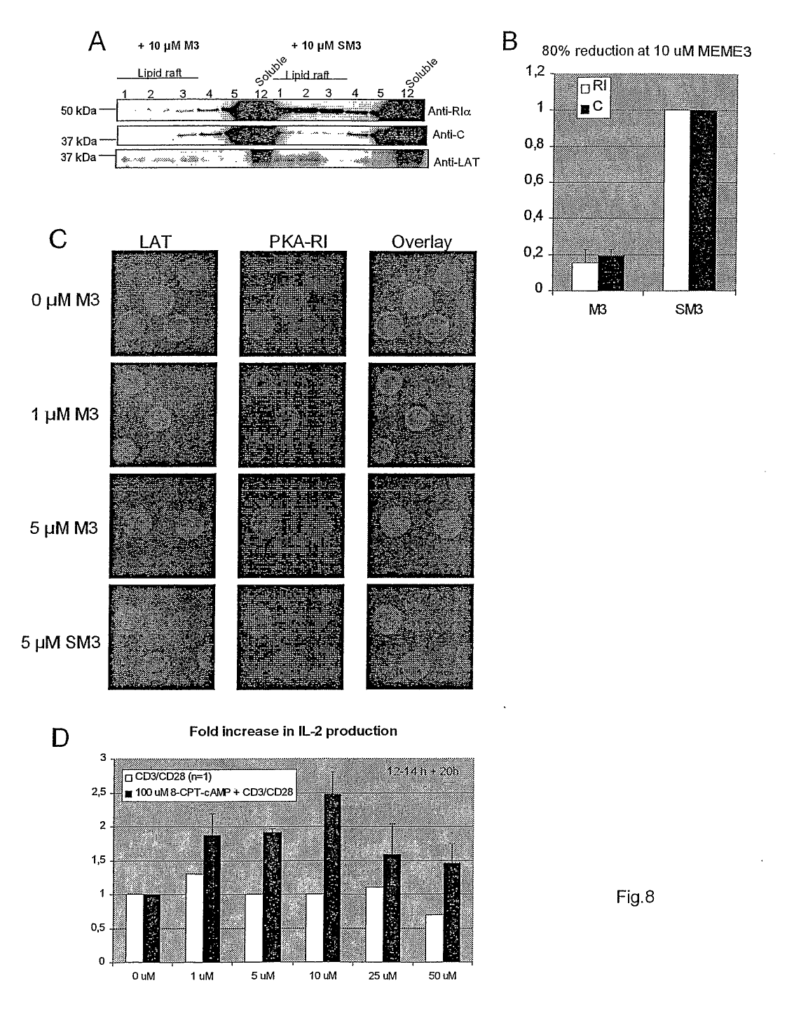

FIG. 8 shows displacement of PKA type I in lipid rafts in T-cells and increased IL-2 production.

(A) Western blot on protein extracts from lipid rafts of human T cells incubation with MEME3 or scrambled MEME3 (SM3) in which lanes 1-5 are lipid raft fractions and lane 12 are non-raft fractions which were analysed with the indicated antibodies.
(B) Results of the densitometry analysis of the results in (A).
(C) Immunofluorescence of human T cells incubated with M3-Arg₁₁ at different concentrations, or SM3, labelled with LAT as a maker of lipid rafts (left hand column), PKA-RI (middle column) and overlap of both (right hand column).
(D) IL-2 production in T cells optionally stimulated with anti-CD3/anti-CD28±the agonist 8-CPT-cAMP in the presence of various concentrations of M3-Arg11.

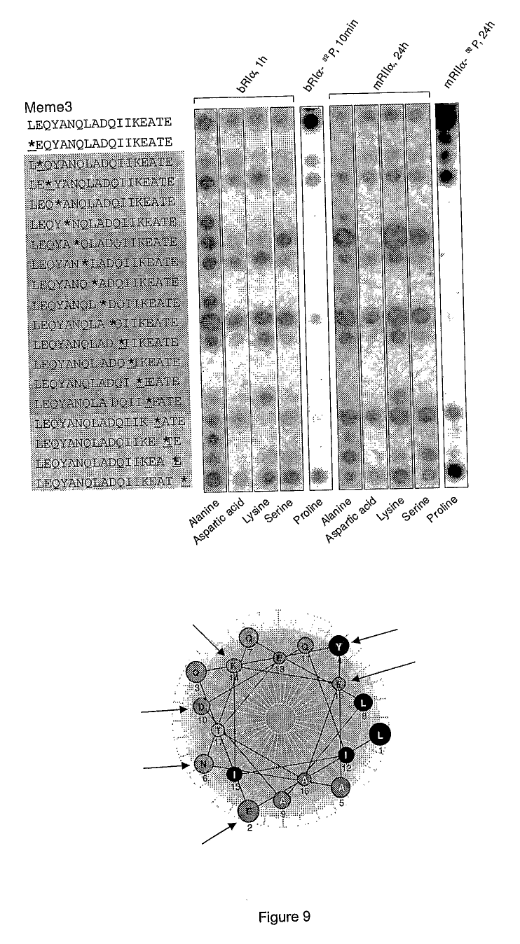

FIG. 9 shows MEME3 substituted with alanine (Ala), aspartic acid (Asp), lysine (Lys), serine (Ser) or proline (Pro) in each position from 1 to 18 (substituted position indicated by asterisks) synthesized on a membrane. R-binding was analyzed by a solid-phase binding assay using ³²P-radiolabeled RIα or RIIα as a probe. Amino acids outside the hydrophobic face important for RIα affinity and specificity in this experiment are indicated by arrows in the helical wheel model of MEME3 (lower panel).

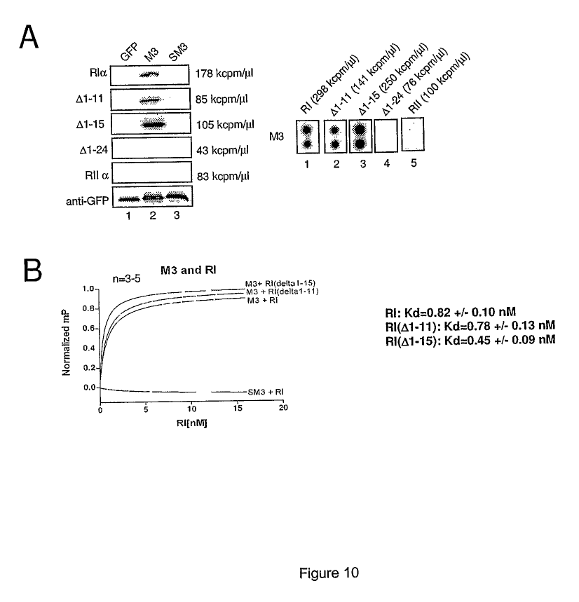

FIG. 10 shows that MEME3 binds with equal or increasing affinity to N-terminal deletion mutants of RIα (Δ1-11 and Δ1-15, but not Δ1-24) compared to the wild type sequence. Lysates from HEK293 cells transfected with GFP, GFP-MEME3 or GFP-SM3 (left panel in A) were analysed for R binding by RIα-³²P- or RIIα-³²P-overlay. Immunoblotting using GFP antibody was used as a loading control. R binding to MEME3 spotted on membrane (right panel in A) was also analysed. Saturation binding curves (in B) were generated using increasing concentrations of RIα wild type protein and the N-terminal RIα deletion mutants. Polarization values (mP) were determined at equilibrium and normalized to the highest value of saturation. Non-linear regression analysis was used to derive K_{d} values from three independent experiments.

FIG. 11 shows that MEME3 synthesized on membrane binds and immobilizes PKA-RIα but not PKA-RIIα from T cell lysates in a solid phase pull down experiment. The presence of the R subunits in the T cell lysate is shown in the left lane. SM3 synthesized on membrane was used as negative control.

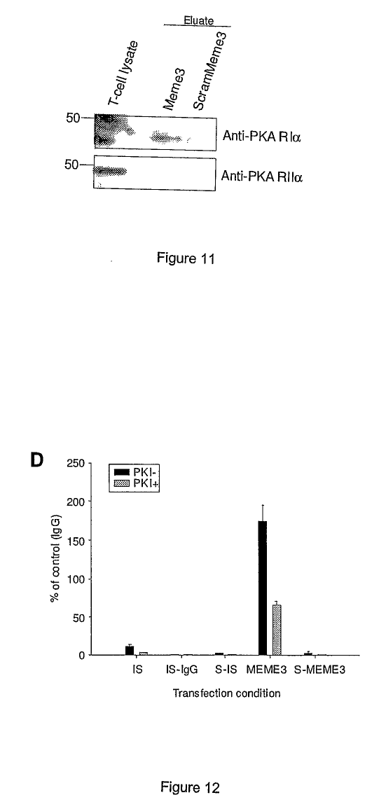

FIG. 12 shows that MEME3 precipitates endogenous PKA kinase activity 17-fold more than the AKAP-is peptide (IS) (RII binding peptide published by Alto et al., 2003, PNAS, 100: 4445-4450) in immunoprecipitation. IgG, scrambled AKAP-is (S-IS) and scrambled MEME3 (S-MEME3) was used as negative controls.

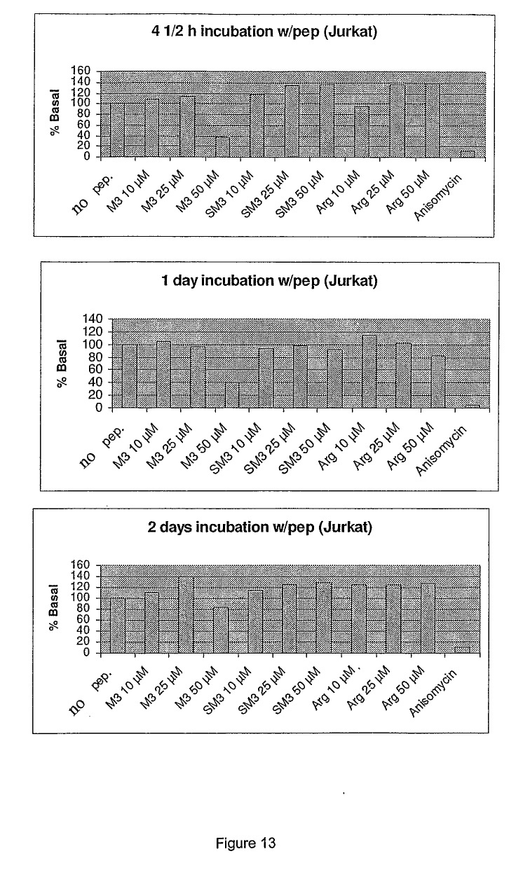

FIG. 13 shows viability of Jurkat T cells treated with different concentrations (0-50 μM) of MEME3-Arg₁₁ and using different incubation times. Anisomycin treated Jurkat T cells were used as a positive control. SM3-Arg₁₁ and Arg₁₁ were used as negative controls.

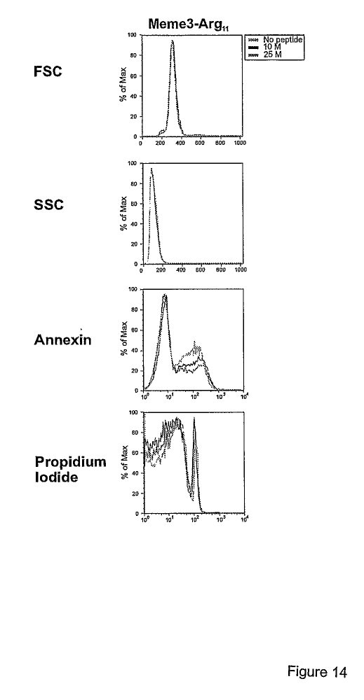

FIG. 14 shows flow cytometric characterization of normal peripheral blood T cells treated with MEME3-Arg₁₁. Activated T cells were incubated with increasing concentrations of MEME3-Arg₁₁ for 40 hours and forward scatter (FSC), side scatter (SSC), Annexin V^{FITC} fluorescence intensity (pre-apoptotic cells) and propidium iodide fluorescence intensity after RNase treatment (DNA content) were also analysed. Data are representative of n=2 experiments. All plots were gated on CD3+ T cells.

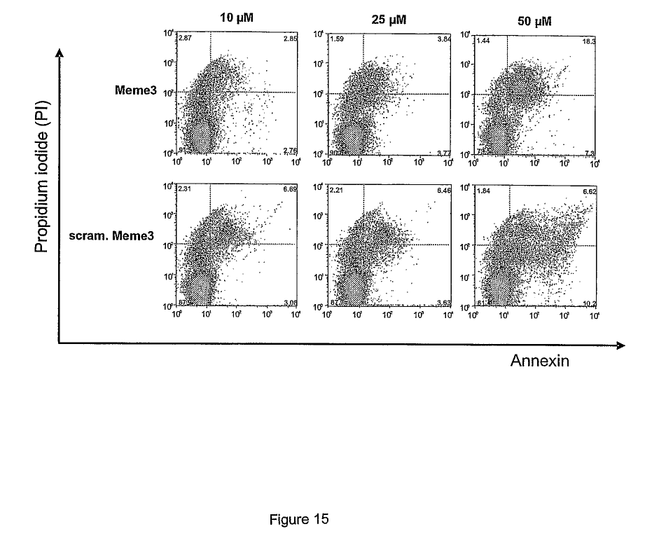

FIG. 15 shows the results of an analysis of cell death monitored by phosphosphingolipid externalization as analysed by Annexin V (AV) binding and disruption of the cellular membrane assessed by PI staining of non-permeabilized cells in cells treated with the indicated peptides at the indicated concentrations. Late apoptotic cells (AV+/PI+) were not detected in samples treated with MEME3-Arg₁₁ or the negative control peptide SM3-Arg₁₁. All plots were gated on CD3+ T cells. Data are representative of n=3 experiments.

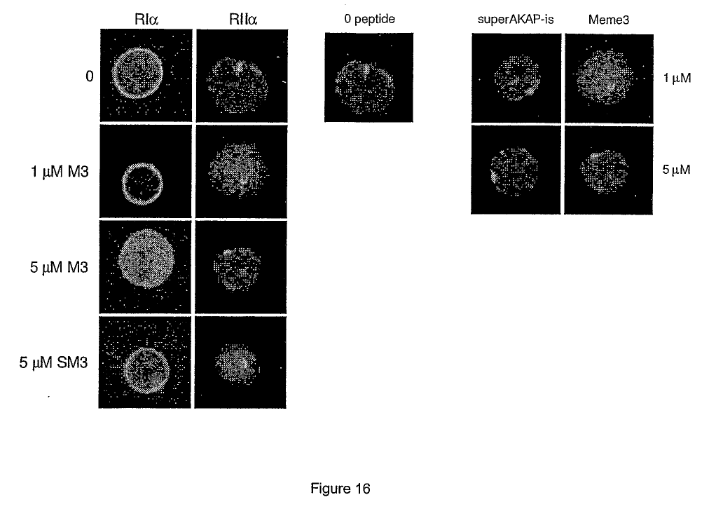

FIG. 16 shows immunofluorescence demonstrating specific disruption of PKA-RIα from anchored sites in the pericortical region (cell periphery) in MEME3-Arg₁₁ treated cells. In contrast, MEME3-Arg₁₁ did not delocalize PKA-RIIα from anchored sites in centrosomes. SM3-Arg₁₁ was used as a negative control. The RIIα specific peptide, Arg₁₁-super-AKAP-is, specifically displaced PKA-RIIα from centrosomes and was used as a positive control for the latter experiment (right panels).

FIG. 17 shows specificity in anchoring disruption by MEME3-Arg₁₁. The PKA-RIIα content in the particulate fraction of T cells treated either with 10-50 μM of MEME3-Arg₁₁ (fractions 2-4) or with 1-50 μM of the RIIα specific peptide, Arg₁₁-SuperAKAPIS (fractions no. 5-8) for 12 h was analysed by immunoblotting. PKCα was used as loading control. The levels of PKA-RIIα were quantified by densitometry of autoradiograms and the results of this analysis are shown in the lower panel. Results shown are means ±SEM from n=3 independent experiments.

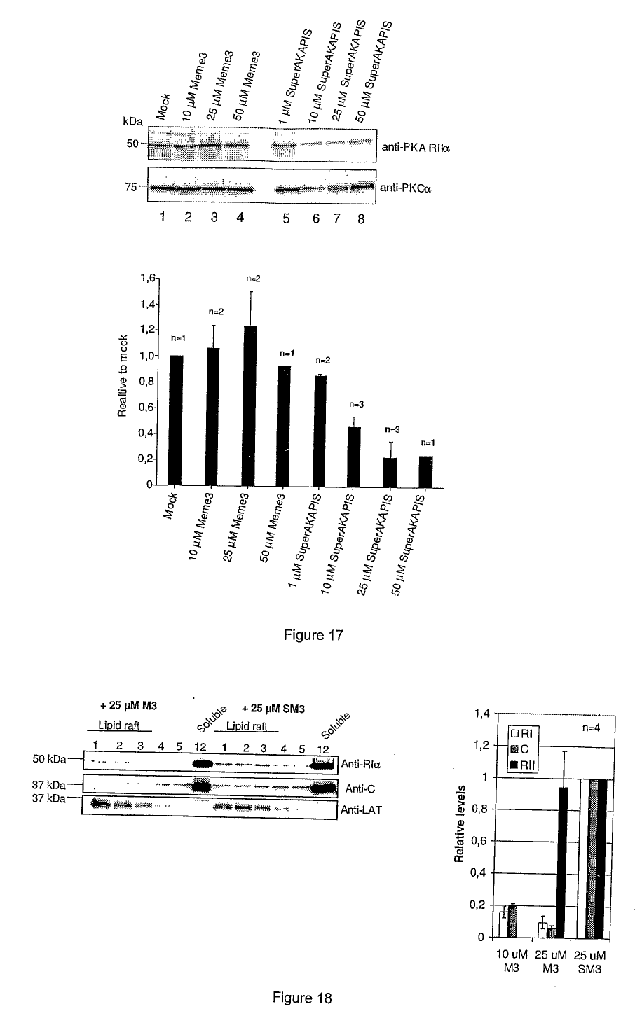

FIG. 18 shows the displacement of PKA-RIα from lipid rafts isolated from human peripheral blood T cells treated with MEME3-Arg₁₁ or the scrambled control sequence SM3-Arg₁₁. The relative levels of PKA-RIα, PKA-C and PKA-RIIα were analysed by immunoblotting (left panel) and measured by densitometry of the autoradiograms (right panel, mean ±SEM, n=4). LAT was used as a marker for lipid raft fractions and was used as an internal standard.

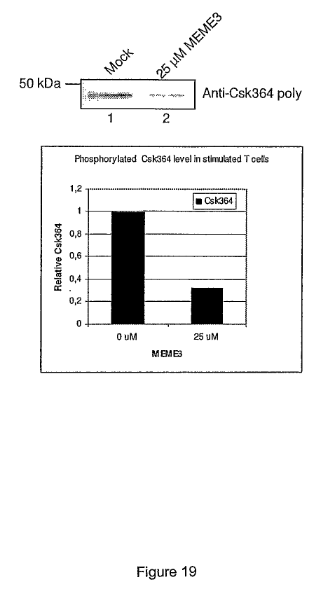

FIG. 19 shows results to assess the level of PKA phosphorylated Serine 364 (PS364) in Csk induced by forskolin treatment of human peripheral blood T cells. The relative level of Csk-PS364 was measured by densitometry after immunoblotting using polyclonal antibody raised towards PS364 in Csk (lower panel) (Yaqub et al., Biochem. J., 372: p 271-8, 2003).

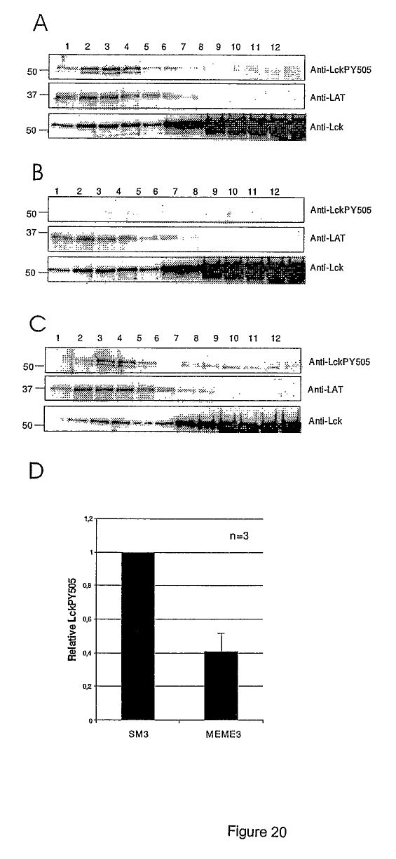

FIG. 20 shows the results if an assay to demonstrate that the level of LckPY505 was greatly reduced in human peripheral blood T cells treated with 25 μM MEME3-Arg₁₁ (in B) compared to untreated cells (in A) or cells treated with the negative control peptide SM3-Arg₁₁ (in C). The relative levels of LckPY505 were measured by densitometry after immunoblotting using specific polyclonal antibodies against LckPY505 (panel D, n=3). LAT was used as a marker for lipid raft fractions and was used as an internal standard.

FIG. 21 shows that MEME3-Arg₁₁ reverses cAMP inhibition of the IL-2 production in human peripheral blood T cells at intermediate concentrations of cAMP (10 μM).

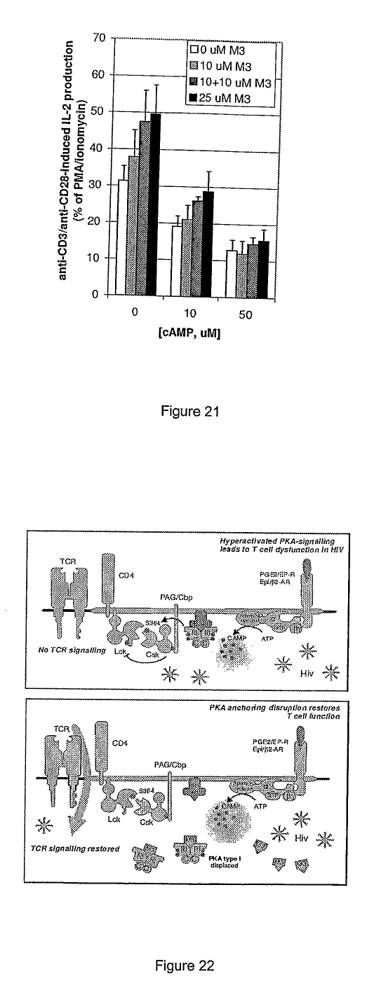

FIG. 22 shows how hyper-activated PKA-signaling may lead to T cell dysfunction in HIV (upper panel), whereas anchoring disruption with MEME3-Arg₁₁ could restore T cell function (lower panel).

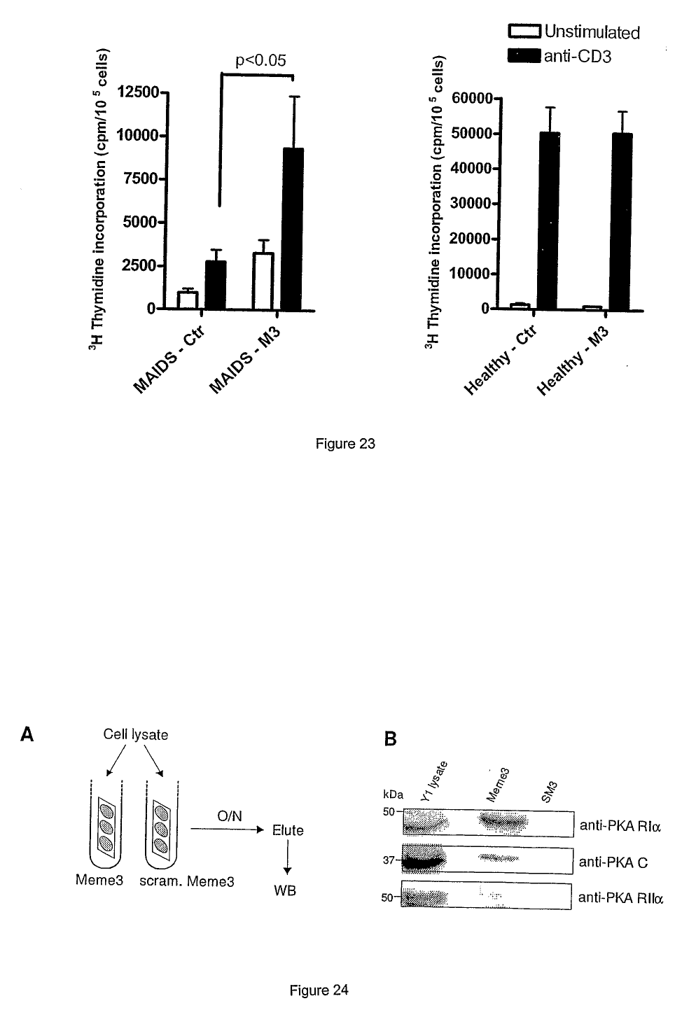

FIG. 23 shows the effect of in vivo treatment of MEME3-Arg₁₁ on T cell immune function of mice with murine AIDS (MAIDS). Mean values ±SEM from each group are shown. Independent samples student T-test revealed a significant increase (p=0.013) in immune response in MAIDS mice injected with MEME3-Arg₁₁.

FIG. 24 shows a schematic representation of a solid phase immobilization assay (A) and levels of PKA-RIα and PKA-C and PKA-RIIα immobilized by MEME3 on solid phase after incubation in Y-1 adrenal cell lysate overnight (B).

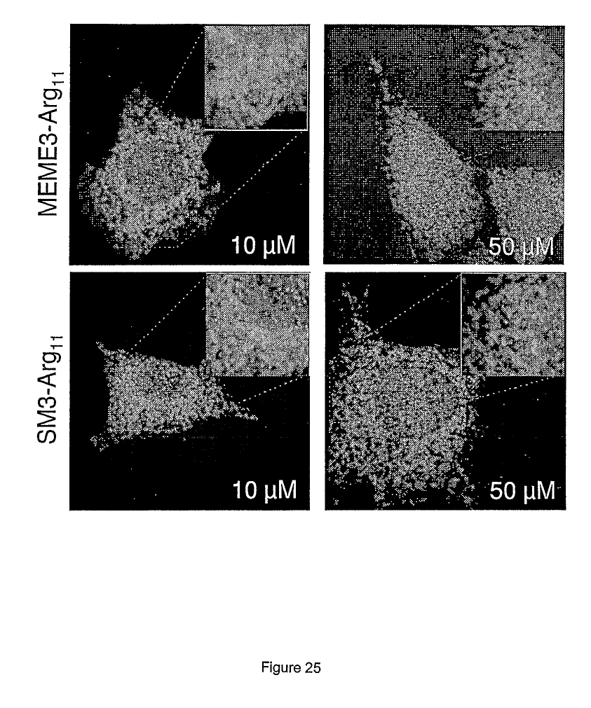

FIG. 25 shows displacement of PKA-RIα (which was stained with an antibody labelled with a green fluorescent dye) from mitochondria (which were visualized by MitoTracker Red) in mouse Y-1 adrenal cells treated with MEME3-Arg₁₁ (10 or 50 μM) for 12 hours (upper panels). SM3-Arg₁₁ was used as negative control (lower panels). There was no yellow co-staining in the MEME3-Arg₁₁ treated cells and this indicates displacement of PKA RIα in these cells.

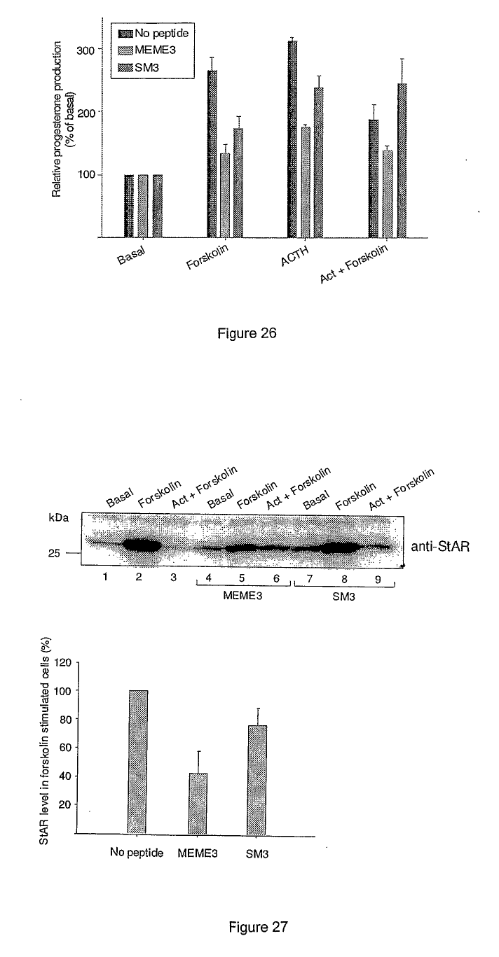

FIG. 26 shows basal level and hormone-stimulated progesterone production in Y1 adrenal cells (relative levels) treated with MEME3-Arg₁₁ or SM3-Arg₁₁ followed by stimulation with ACTH or forskolin.

FIG. 27 shows the level of StAR protein in cells that were treated with MEME3-Arg₁₁ or SM3-Arg₁₁ followed by stimulation with ACTH or forskolin. The relative StAR level in forskolin-stimulated cells is shown in the lower panel. The densitometry analysis data shown in the lower panel are representative of three separate experiments. Error bars in the graph indicate the standard error of mean (SEM).

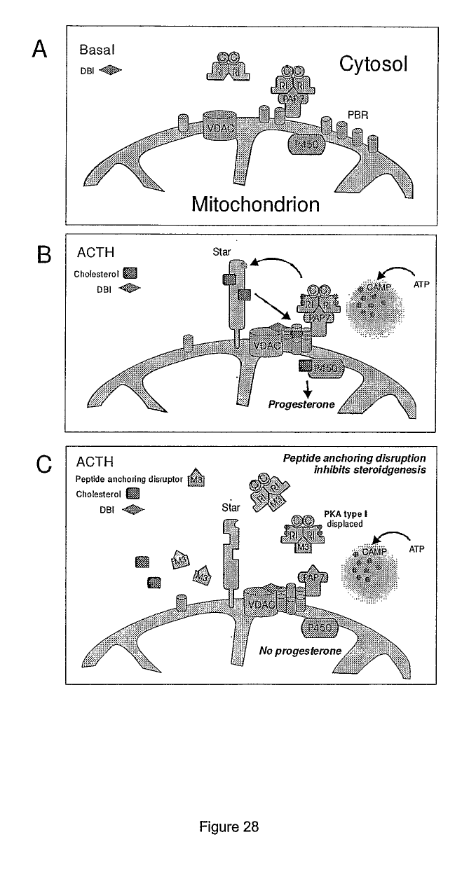

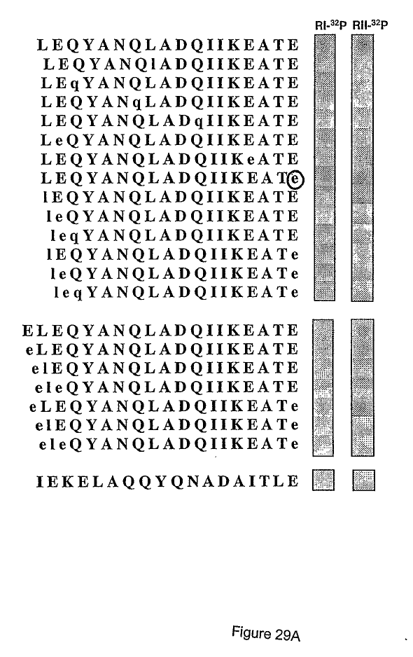

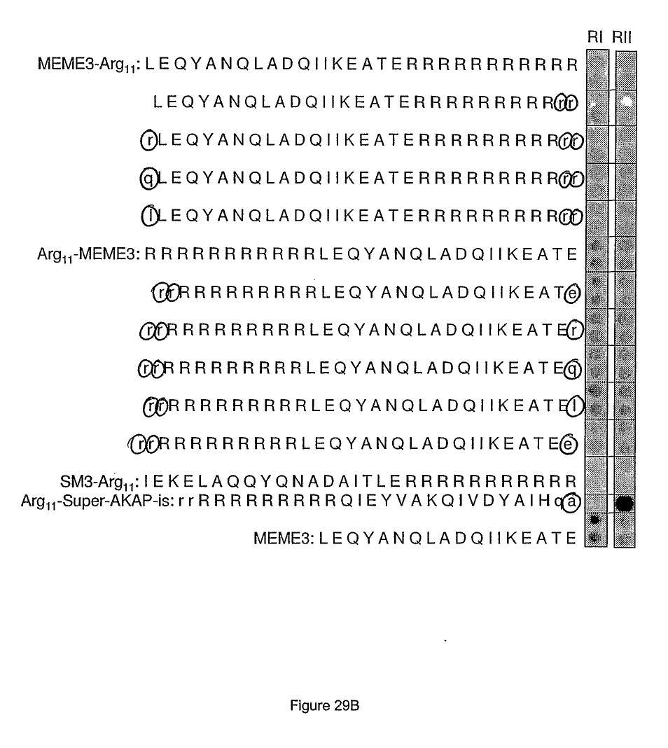

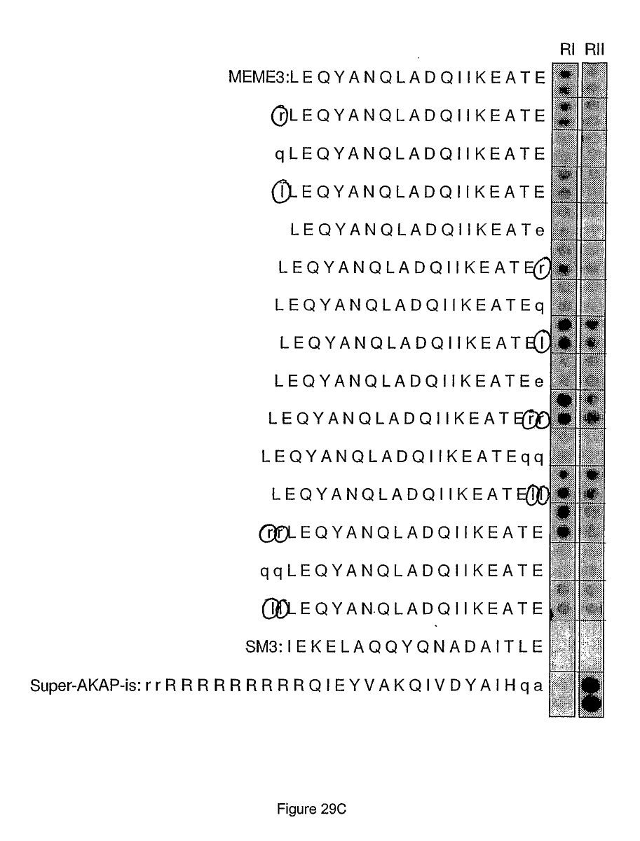

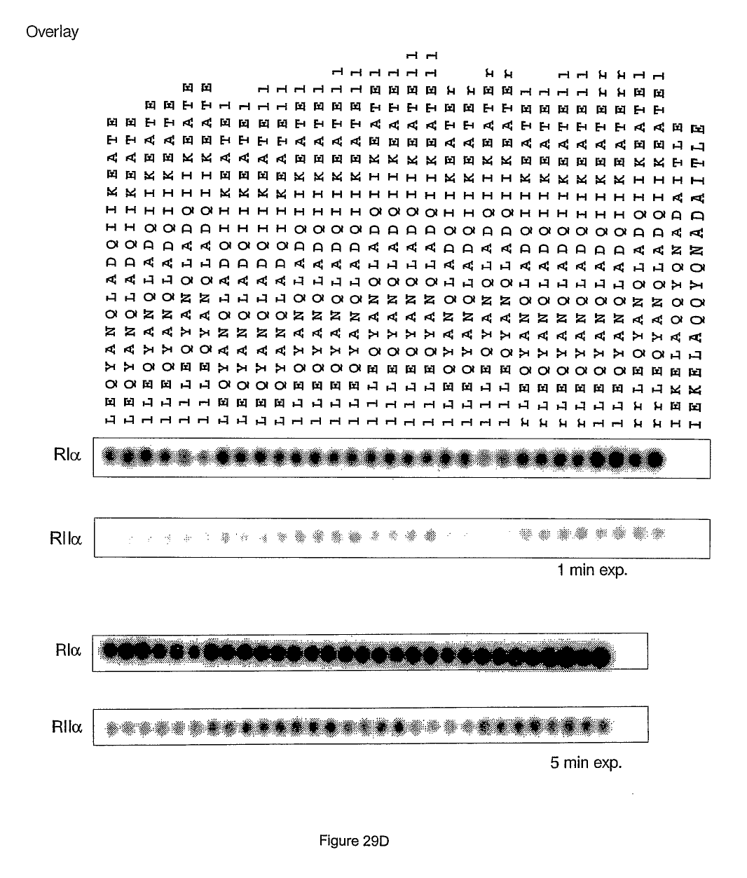

FIG. 28 shows a schematic diagram illustrative the involvement of PKA type I signaling pathway and the effect of anchoring disruption in the regulation of StAR phosphorylation and steroidgenesis. The model is a modified and extended version of the figure published by Liu et al., (2003, J Steroid Biochem Mol Biol, 85: 275-283) (A) In the basal situation, the StAR level is low and no pregnenolone is produced. (B) Upon hormonal stimulation, PKA anchored to the AKAP Pap7 phosphorylates and activates newly synthesized StAR, which then increases the transport of cholesterol over the mitochondrial membrane. Cholesterol is next converted to pregnenolone by P450ssc (side chain cleavage enzyme) in the rate limiting first step in steroid biosynthesis. (C) The PKA-PAP7 interaction is disrupted by treatment with MEME3-Arg₁₁ resulting in no phosphorylation of the StAR protein upon hormonal stimulation. Lack of StAR activity leads to inhibited steroid production.

FIG. 29 shows R-binding to MEME3 derivatives with D-amino acid substitutions (in small letters) or with additional D-amino acids at the N- or C-terminus synthesized on membrane. R-binding was analyzed by a solid-phase binding assay using ³²P-radiolabeled RIα or RIIα (in A-D).

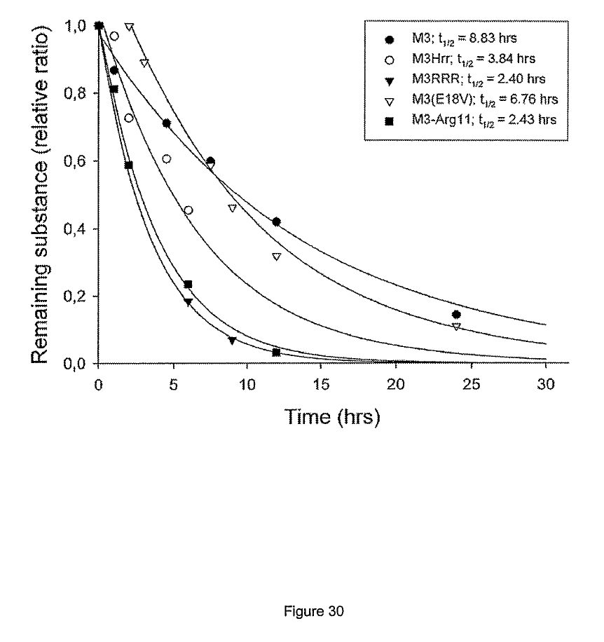

FIG. 30 shows stability of MEME3 and stability of peptide derivatives with D-glutamic acid substitutions in 10% human serum. Indicated peptides and peptidomimetics were incubated in 10% human serum for indicated periods of time and amounts of remaining full-length peptide was determined by reverse phase, high pressure liquid chromatography (HPLC) and peptide half lifes calculated.

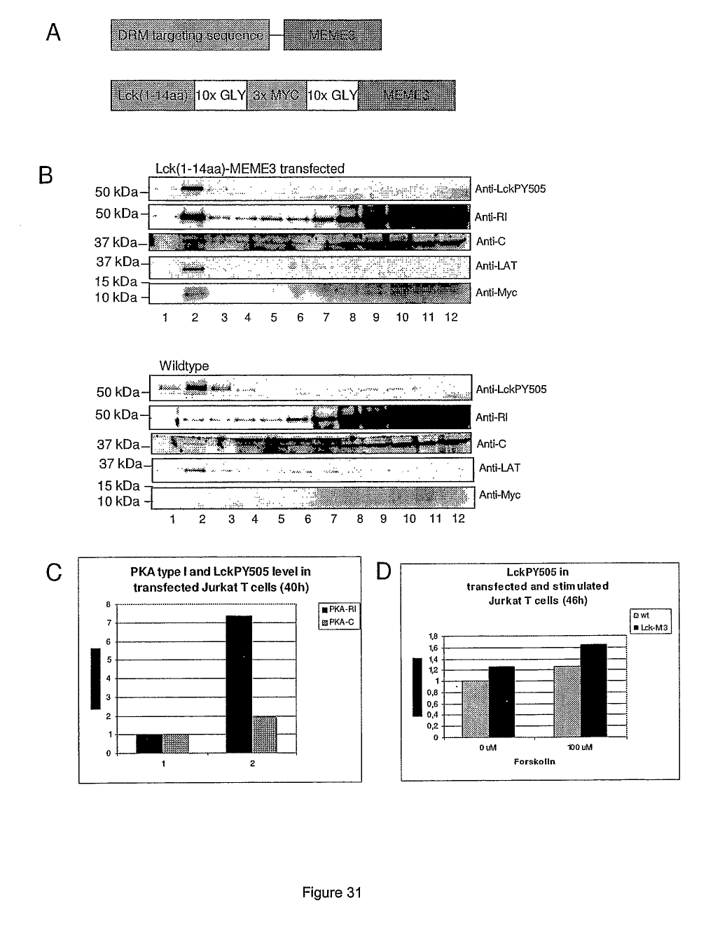

FIG. 31 shows that enhanced PKA type I signaling at a defined subcellular site may be achieved by MEME3-mediated targeting of PKA type I, here to lipid rafts or DRMs (detergent resistant membranes). A schematic illustration of a construct that potentially can be used to target PKA type I to lipid rafts and enhance PKA signaling locally is shown in A. Lipid rafts were isolated from Jurkat T cells transfected with such a construct (lower construct in A), followed by analysis by 4-20% PAGE and blotting onto PVDF membranes. Subsequently, the levels of Myc, PKA-C, PKA-RIα, LckPY505 and LAT were analysed by immunoblotting (B). The relative levels of PKA-C and PKA-RIα were measured by densitometry analysis of the autoradiograms and normalized to levels of the lipid raft marker LAT (C). LckPY505 was analysed as a measure of Csk activity (and indirectly of PKA type I activity as PKA activates Csk) (D).

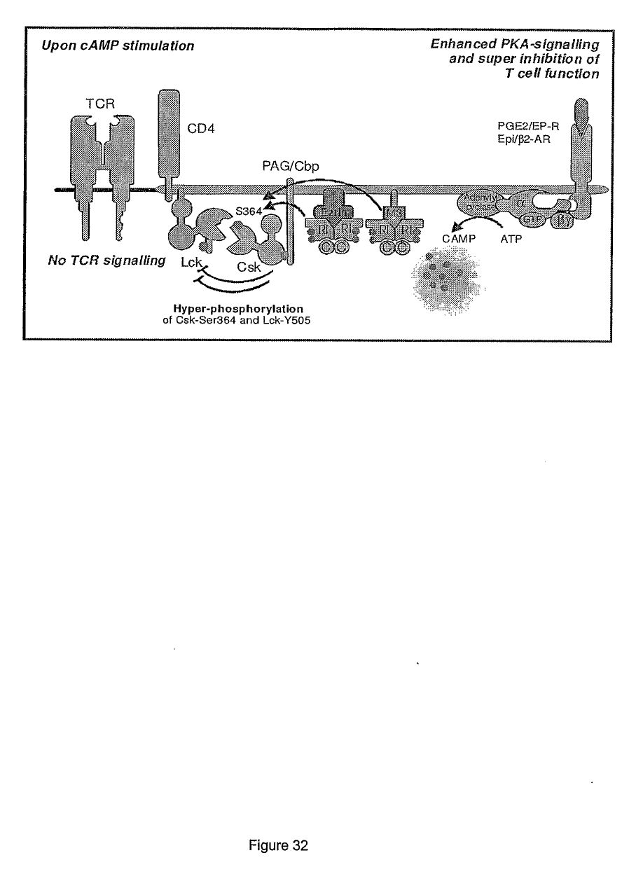

FIG. 32 shows a schematic diagram which illustrates how MEME3 targeted to the lipid rafts may recruit PKA type I which again leads to PKA-hyper-phosphorylation of Csk followed by increased LckPY505 and abrogated TCR signaling.

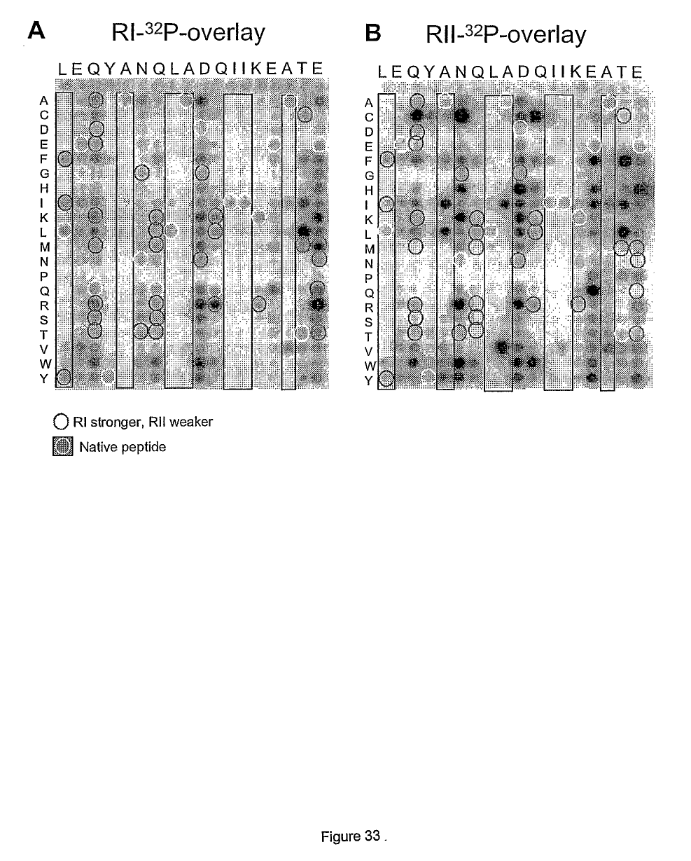

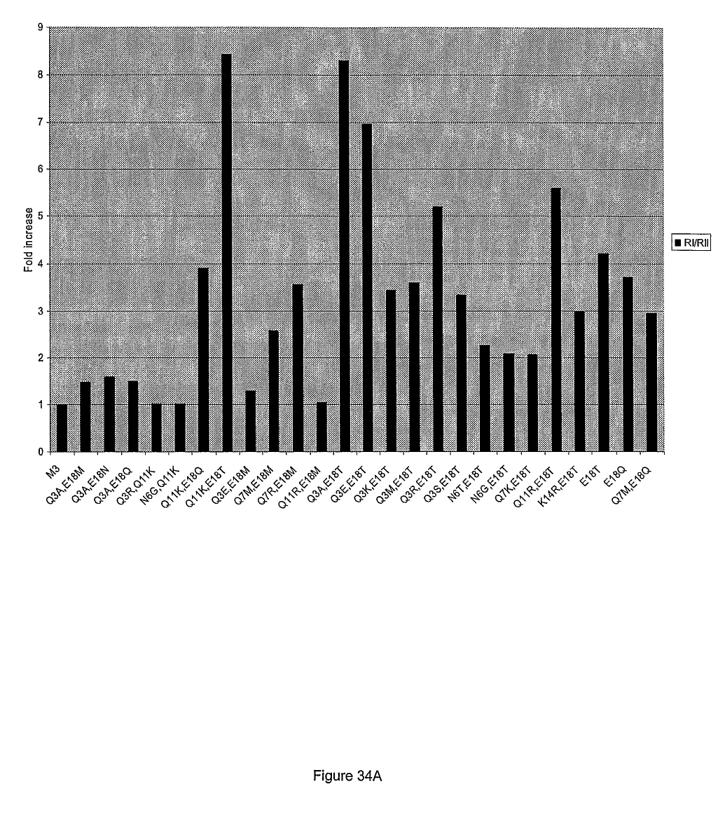

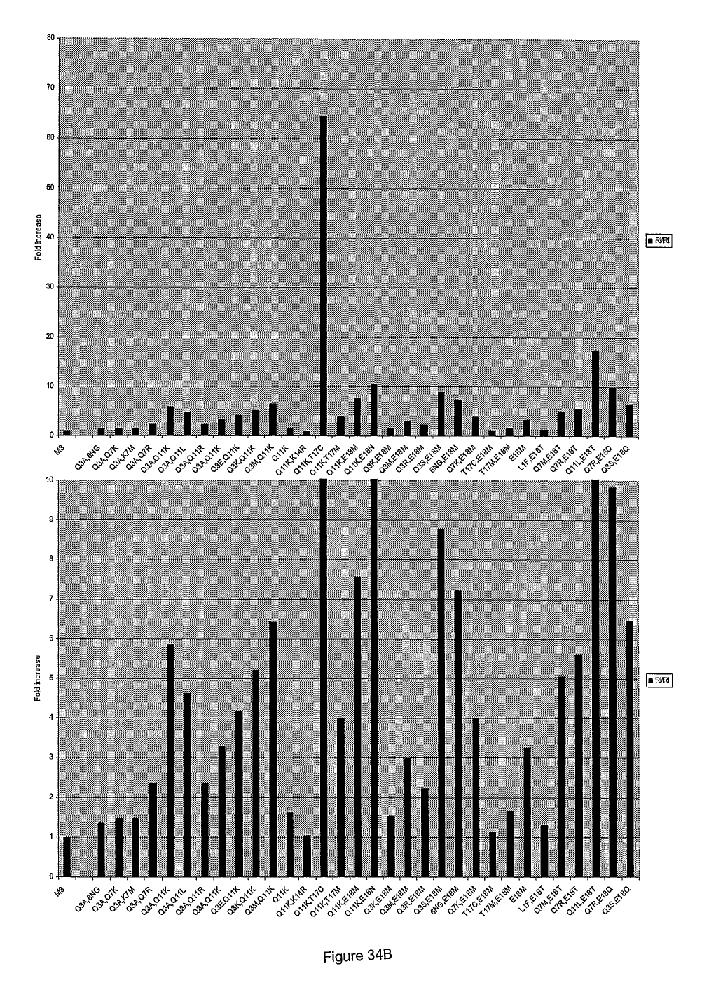

FIG. 33 shows further optimisation of MEME3. A two-dimensional array of 360 MEME3 peptide derivatives was synthesized (Multipep automated peptide synthesizer, INTAVIS Bioanalytical Instruments AG, Koeln, Germany) where each residue in MEME3 (given by their single-letter codes above each array) was replaced with residues having every possible side chain (given by their single-letter codes to the left of each array). The first row in each array corresponds to the native peptide (MEME3). The MEME3 derivatives were analyzed for R binding by either (A) RIα-³²P- or (B) RIIα-³²P-overlay. Binding of ³²P-labeled RIα (A98S) or RIIα was detected by autoradiography. The position of hydrophobic amino acids at positions 1, 5, 8, 9, 12, 13 and 16 are indicated by boxes. MEME3 derivatives with higher RIα affinity and lower RIIα affinity are indicated by circles. White circles denote peptides in the array that correspond to the native MEME3 sequence.

FIG. 34 shows selected modified MEME3 derivatives (single and double substitutions) with a higher RIα specificity than MEME3 (A-C).

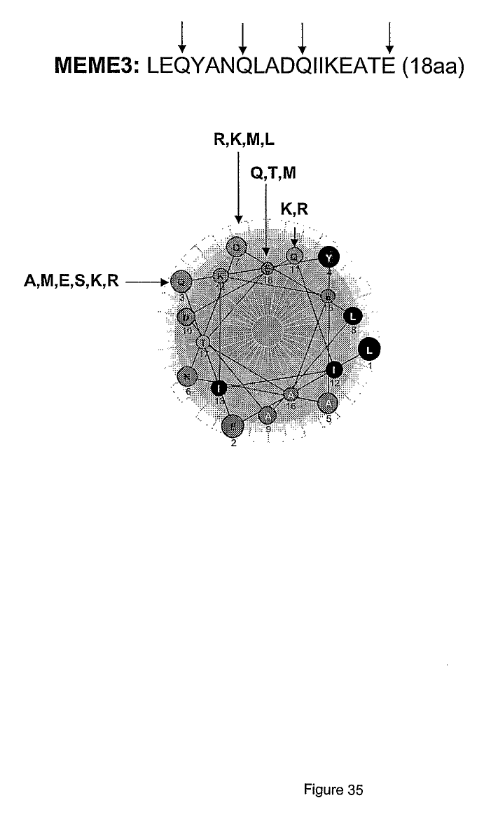

FIG. 35 indicates the most preferable positions for substitutions of MEME3 and the most preferred substitutions. MEME3 is depicted both as a linear sequence and as in an α-helical configuration (helical wheel model) where preferred substitutions are marked with arrows.

### EXAMPLES

### Methods

Autospot Peptide Array. Peptide arrays were synthesized on cellulose paper by using an Autospot Robot ASP222 (ABiMED, Langenfeld, Germany) as described (Frank, R., 1992, Tetrahedron 48, 123-132) or Multipep automated peptide synthesizer (INTAVIS Bioanalytical Instruments AG, Koeln, Germany) as described (Frank, R., 1992, Tetrahedron 48, 123-132).

MEME software. MEME software was used for consensus sequence generation (http://meme.sdsc.edu) (Grundy, W. N., et al., 1997, Comput. Appl. Biosci. 13, 397-406). MEME setting included one motif per sequence, and a motif length of 20 aa was specified.

R-overlay. R-overlays were conducted as described, using ³²P-labeled recombinant murine RII (Hausken, Z. E., et al., 1998 in Protein Targeting Protocols, ed. Clegg, R. A. (Humana, Totowa, N.J.), Vol. 88, pp. 47-64) or recombinant bovine RI (A98S) or by cold RI-overlay where bound RIα was detected by Western blot. Cold RI-overlay was performed by incubating the membranes with 250 nM RI overnight at 4° C., then the membranes were washed five times in TBST for 5 min. Detection of RI was performed using an anti-RI rabbit polyclonal antibody (Santa Cruz) at a concentration of 0.2 μg/μl for 1 hour at 4° C. Then the membranes were washed five times for 5 minutes in PBS followed by incubation with HRP-conjugated anti-rabbit IgGs (1:5000) for 1 hour at 4° C. The membranes were washed in PBS as described above before detection by Supersignal West Dura Extended Duration Substrate (Pierce).

Densitometric analysis. The densitometric analysis was performed using Scion Image (www.scioncorp.com) (Scion Coorperation, Mo., USA) or Quantity One version 4.5.0, (BioRad).

Constructs and mutagenesis. Oligonucleotides with the following sequences were directionally cloned into EGFP-C (Clonetech) vector via TOPO blunt vector (Invitrogen) by using SalI and BamHI sites. MEME3(+), 5′-GTC GAC-CTG GAG CAG TAC GCC AAC CAG CTG GCC GAC CAG ATC ATC AAG GAG GCC ACC GAG-GGA TCC-3′; MEME3 (−), 5′-GGA TCC-CTC GGT GGC CTC CTT GAT GAT CTG GTC GGC CAG CTG GTT GGC GTA CTG CTC CAG-GTC GAC-3′; Scramble (+), 5′-GTC GAC ATC GAG AAG GAG CTG GCC CAG CAG TAC CAG AAC GCC GAC GCC ATC ACC CTG GAG GGA TCC-3′; and Scramble (−), 5′-GGA TCC CTC CAG GGT GAT GGC GTC GGC GTT CTG GTA CTG CTG GGC CAG CTC CTT CTC GAT GTC GAC-3′. The site specific mutation, A98S, was made in RI wild type cloned into pRSETB (Invitrogen) using a site specific-mutagenesis kit (Stratagene). RI( 1-11), RI(Δ1-15) and RI(Δ1-24) mutants were made by PCR and re-cloned into pRSETB (Invitrogen). All constructs were confirmed by sequencing.

Fluorescence Polarization. FITC-labeled peptides (SynPep, San Diego) that were used for fluorescence polarization studies included MEME3 (LEQYANQLADQIIKEATEK(5-Carboxyfluorescein)-CONH2) and MEME3 scrambled peptide (IEKELAQQYQNADAITLEK(5-Carboxyfluorescein)-CONH2). Peptides (0.5 nM for RI experiments and 1 nM for RII experiments) were suspended to working dilutions in phosphate-buffered saline containing 5 μg/μl BSA, pH 7.0. Increasing concentrations of recombinant bovine RI wt or recombinant murine RII were added to a PBS solution and mixed with each FITC-labeled peptide. Each sample was incubated for 2.5 hours. Fluorescence polarization was measured on a Beacon 2000 (Panvera, Madison, Wis.), following the manufacturer's instructions. Saturation binding curves were generated with PRISM graphing software (GraphPad, San Diego). Dissociation constants (K_{d}) were calculated from the nonlinear regression curve from averages of minimum three individual experiments.

Cell cultures and transient transfections. The human leukemic T cell line Jurkat (clone E6.1), Jurkat TAg, a derivative of the Jurkat cell line stably transfected with the SV40 large T antigen (Clipstone, N. A., and Crabtree, G. R., 1992, Nature. 357:695-697) was cultured at 37° C. in RPMI medium (Gibco BRL) supplemented with 10% fetal calf serum, 2 mM glutamine, penicillin, streptomycin, 1 mM sodium pyruvate and nonessential amino acids. For transfections, cells (5×10⁶) in 0.4 ml Opti-MEM were mixed with 40 μg of each DNA construct (GFP-MEME3 or GFP-Scrambled MEME3) in electroporation cuvettes with a 0.4-cm electrode gap (BioRad Laboratories) and subjected to an electric field of 250 V/cm with 960 μF capacitance. A total of 2×10⁷ cells were transfected per DNA construct. The cells were expanded in complete medium and harvested after 20 h. The transfection efficiency was determined by microscopic analysis.

HEK293 cells at 50-800 confluency were transfected with ten μg of plasmid DNA (GFP, GFP-MEME3 or GFP-Scrambled MEME3) by using the CaCl₂ method. Cells were lysed after 30 hours in lysis buffer (20 mM Hepes, pH 7.5/150 mM NaCl/1 mM EDTA/1% Triton X-100) with protease inhibitors (Complete Mini, EDTA-free tablets, Roche).

Peripheral blood CD3⁺ T cells were purified by negative selection from 50 ml of heparin-treated blood from normal, healthy donors (Ullevaal University Hospital Blood Center, Oslo, Norway). Briefly, peripheral blood mononuclear cells were isolated by density gradient (Lymphoprep, NycoMed, Oslo, Norway) centrifugation, followed by negative selection using monodisperse magnetic beads directly coated with antibodies to CD14 (Dynabeads M-450 CD14, 111.12) and CD19 (Dynabeads M-450 CD19, 111.04) and a magnet. All steps were performed at 4° C. Cell suspensions were routinely screened by flow cytometry and shown to consist of more than 90% CD3.

Mouse adrenocortical Y1 cells were obtained from ATCC (Cat. No. CCL-79) and maintained in DMEM/HAM'S F-12 (Cat. No. E15-813, PAA Laboratories GmbH, Pasching, Austria) supplemented with 100 μg/ml streptomycin, 100 U/ml penicillin and 10% (v/v) fetal calf serum in a humidified atmosphere of 5% CO₂ and split by trypsination at less than 80% confluence.

Immunoblot analysis. Immunocomplexes and lipid raft fractions were analysed on a 10, 15% or 4-20% PAGE and blotted onto PVDF membranes. The filters were blocked in 5% non-fat dry milk in TBST for 30 minutes at RT, incubated 1 hour at RT (or overnight at 4° C.) with primary antibodies, washed four times 5 minutes in TBST with 0.1% Tween-20 and incubated with a horseradish-peroxidase-conjugated secondary antibody. Blots were developed by using Supersignal West Dura Extended Duration Substrate or Supersignal West Pico substrate (Pierce).

Antibodies. RI or RII were immunoprecipitated with polyclonal antibodies against human RI or human RII at a 3 μg/ml dilution (Santa Cruz Biotechnology). MEME3 or scrambled peptide was immunoprecipitated with 1.5 μl of anti-GFP antiserum (Invitrogen). Monoclonal (Transduction laboratories) or polyclonal antibodies (Santa Cruz Biotechnology) against human RIα and RIIα were used at a 1 μg ml⁻¹ or 0.5 μg ml⁻¹ ilution for Western blotting. Polyclonal antibodies against human Cα (Santa Cruz Biotechnology) and LAT (Upstate Biotechnology) were used at a 0.4 μg ml⁻¹ and a 1 μg ml⁻¹ dilution for Western blotting, respectively. Polyclonal antibodies against LckPY505 (Cell Signaling) were used at a 1:1000 dilution and polyclonal antibody raised towards PS364 in Csk were used as previously described (Yaqub et al., Biochem. J., 372: p 271-8, 2003). Polyclonal antibodies against StAR (Affinity BioReagents) were used at 1 μg ml⁻¹. For PKA kinase activity assay monoclonal antibody against the V5 epitope was used accordingly to the manufacturer (Invitrogen). Monoclonal and polyclonal antibodies against GFP were used at a 1 μg ml⁻¹ dilution for Western blotting (Clontech). HRP-conjugated anti-mouse or anti-rabbit IgGs were used as secondary antibodies at a 1:10,000 dilution (Jackson Immunoresearch).

Blots were developed by using Supersignal West Pico substrate (Pierce). For immunofluorescence polyclonal and monoclonal antibodies against RIα (Biogenesis), RIIα (Transduction laboratories) and AKAP450 (A24, Tasken et al., 2001, J. Biol. Chem, 276:21999-22002) were used as primary antibodies. Secondary antibodies (all Molecular Probes) were Alexa-488 conjugated anti-mouse and anti-rabbit IgG (both goat) and Alexa-555 anti-rabbit IgG (goat).

Protein expression and purification. Human RI, Bovine RI and N-terminal deletions thereof or murine RII was expressed in E. coli B121 by IPTG induction. The RI containing pellet was incubated in lysis buffer (10 mM MOPS, pH 6.5, 100 mM NaCl, 10 μM DTT added protease inhibitors) and sonicated (Branson Sonifier 250) for 1 minute in three intervals at 0° C. After centrifugation, the supernatant was incubated with CAMP beads (Sigma, A-0144) and rotated overnight at 4° C. The RI protein bound to the beads was washed two times in lysis buffer thereafter three times in the washing buffer (5 mM MOPS, pH 6.5, 0.5 M NaCl, 5 μM DTT) and finally two times in lysis buffer. The RI protein was eluted in 25 mM CAMP (dissolved in the washing buffer containing high salt, 1 M NaCl) at RT for 1 hour before dialysis into PBS overnight. Murine RII was purified by the HIS tag using FPLC.

Peptide synthesis and loading. MEME3, SMS (scrambled MEME3), Super-AKAP-is and scrambled Super-AKAP-is were synthesized plain or with eleven arginine residues at the Super-AKAP-is N- or C-terminus and purified to >80% purity (SynPep, San Diego, USA or in house synthesizer at the Biotechnology Centre of Oslo). The arginine-coupled peptides were added directly to the cell culture (1-5 μM or 0-50 μM) at different times and incubated overnight before the cells were harvested and analysed further, e.g. by immunofluorescence.

### MEME3: LEQYANQLADQIIKEATE

### SM3: IEKELAQQYQNADAITLE

### SuperAKAPIS: QIEYVAKQIVDYAIHQA

### Scrambled SuperAKAPIS: QDVEIQLKAAYNQKLIAI

Displacement of PKA-RII from the particulate Fraction in T-cells. Negatively selected CD3⁺ T-cells (20×10⁶/3 ml/well) were cultured in 6-well plates with 0-50 μM Arg₁₁-SuperAKAPIS (or 0-50 μM M3-Arg₁₁) for 12 hours. Cells were lysed in 250 μl lysis buffer (50 mM Tris pH 7.4, 100 mM NaCl, 5 mM EDTA, 50 mM NaF, 10 mM Na-pyrofosfat, 1 mM Na₃VO₄, 1 mM PMSF, 0.7% triton X-100) and centrifuged at 14 000×g for 10 min. The sedimented proteins were resuspended in loading dye before being resolved by SDS/PAGE and detected by immunoblotting.

Flow cytometry. Negatively selected CD3⁺ T cells cultured in flat-bottom, 96-well plates (Costar; 0.2×10⁶ cells/well) were activated with OKT-3 (4 ng/μl) for min at 37° C., crosslinked with F(ab′)₂ fragments (39 μg/ml) and thereafter incubated with arginine-coupled peptides at 0-50 μM. For analyzing the DNA content, the cells were incubated further for 36-40 hours and thereafter washed in PBS, fixed in 10 paraformaldehyde, permeabilized in FACS Permeabilizing Solution (BD BioSciences, San Jose, Calif.) for 10 min and washed in PBS containing 1% BSA and 100 μg/ml RNase prior to being stained with propidium iodide (PI, Cat. No. 51-66211E BD Biosciences Pharmingen, San Diego, Calif., USA) (25 μg/ml) for 15 min 37° C. The cells were washed twice in PBS and fixed in 1% paraformaldehyde before flow cytometry (FACSCalibur, BD Biosciences, San Jose, Calif.) and subsequent analysis using FlowJo software (Tree Star, San Carlos, Calif.). For analyzing apoptosis and necrosis, the cells were incubated further for 18 hours and thereafter washed in PBS, fixed in 1% paraformaldehyde and washed with annexin binding buffer (Cat. No. 51-66121E, BD Biosciences) prior to being stained with Annexin V (3 μl to 50 μl volume, Cat. No. 556419, BD Biosciences Pharmingen) and PI (3 μg/ml) for 20 min on ice. The cells were washed twice in PBS and fixed in 1% paraformaldehyde before being analyzed on a flow cytometer (FACSCalibur, BD Biosciences, San Jose, Calif.) and analyzed using FlowJo software (Tree Star, San Carlos, Calif.).

Cell viability/Metabolic activity. A WST-1 (a sodium salt of 4-[3-(4-iodophenyl)-2-(4-nitrophenyl)-2H-5-tetrazolio]-1,3-benzene disulfonate) assay (Cat. No. 1644807, Roche, Mannheim, Germany) was performed to assess the effect of the various peptides on cell viability. The assay was performed according to the instructions of the manufacturer. In brief, 25 000 Jurkat cells/well in a 96-well plate with 100 μl cell culture medium were incubated with or without peptides for 4½ h, 24 h or 48 h before 10 μl cell proliferation agent WST-1 was added to each well. The cells were then incubated for another 2 h at 37° C. in a 5% CO₂ atmosphere. After 1 min of shaking, the multiplate was placed in an ELISA multiplate reader (Sunrise, Tecan) and measured against a background control consisting of 100 μl medium and 10 μl cell proliferation reagent WST-1 at a wavelength of 420 nm.

### Immunofluorescence and Confocal Microscopy.

Immunofluorescence analysis of cells was performed as previously described (Collas, P. et al., 1996, J. Cell Biol. 135, 1715-1725). RI was detected by using an anti-RI monoclonal antibody (Transduction Laboratories) as a primary antibody at a 2.5 μg/μl dilution and Alexa-488 as a secondary antibody (1:600, Goat anti Mouse IgGs, Molecular Probes). Immunofluorescence was performed on Y1 cells attached to collagen/fibronectin coated coverslips. Mitochondria were stained using MitoTracker Red CMXRos (Molecular Probes) in fresh medium with incubation at 37° C. for 30 min prior to cell fixation. All cells were washed in PBS, fixed with 3% paraformaldehyde and permeabilised using 0.1% Triton X-100. Proteins were blocked in 2% BSA/PBST prior to antibody labeling. Primary and secondary antibodies were used at a 1:200 dilution in BSA/PBST and incubated for 30 min. DNA was counterstained using TO-PRO 3 (Molecular Probes) at a concentration of 1 μM. Cover slips were mounted with ProLong Gold antifade reagent (Molecular Probes) and cells examined in a Nikon Diaphot 300 microscope equipped for laser scanning confocal microscopy (BioRad MRC 1000/1024). A 60×/1.4 oil objective was used. Alternatively, fluorescently labeled cells were examined using a Leica TCS SP1 confocal fluorescence microscope (Leica, Heidelberg, Germany) equipped with an Ar (488 nm) and two He/Ne (543 and 633 nm) laser lines. A Plan apochromat 100×/1.4 oil objective was used. All multi-labeled images were acquired sequentially and exported as TIF files for image preparations using CorelDraw/Photo-Paint 12 (Corel Corp., Ontario, Canada).

Lipid Raft Purification. Isolation of lipid rafts or glycolipid-enriched membrane microdomains was performed as described in detail elsewhere (Zhang, W. et al., 1998, Immunity 9:239-246). In brief, cells were homogenized in 1 ml ice-cold lysis buffer (50 mM Hepes, pH 7.4, 100 mM NaCl, 5 mM EDTA, 0.70 Triton X-100, 10 mM sodium pyrophosphate, 1 mM Na₃VO₄, 50 mM NaF, 1 mM PMSF, and protease inhibitors) by 10 pestle strokes in a Dounce homogenizer, loaded at the bottom of a 40-5% sucrose gradient and centrifuged at 200,000 g for 5 or 20 h. 80 μl or 0.4-ml fractions were collected from the top. The fractions were analysed by immunoblot analysis. LAT was used as a marker for lipid raft fractions.

Coimmunoprecipitation of PKA kinase activity. Cells at 50-80% confluency were transfected by using FuGENE (Roche Biochemicals). Five micrograms of plasmid DNA (GFP-AKAP-is-V5His, GFP-Scrambled-AKAP-is-V5His, GFP-MEME3-V5H is-and GFP-Scrambled-MEME3-V5His) was transfected into HEK293-cells. Cells were lysed 24 h later in 20 mM Hepes, pH 7.5/150 mM NaCl/1 mM EDTA/1% Triton X-100. MEME3 and the other peptides was immunoprecipitated. PKA kinase assays were performed by the filter paper assay (Corbin and Reimann, 1974, Methods Enzymol., 38, 287-294). The protein kinase inhibitor (PKI) residues 5-24 peptide was used as a specific inhibitor of the kinase (Scott et al., 1986, PNAS, 83, 1613-1616).

Progesterone production and StAR protein level. Y1 cells were cultured overnight in 6-well plates at the density of 1.5×10⁶ cells per well. To increase the basal level of StAR, the cells were pre-stimulated with 10 μM forskolin for 1 hour, washed twice with medium, before peptide loading. Thereafter, the cells were loaded with 50 μM of the arginine-coupled peptide for 5 hours before being treated with 5 μg/ml Actinomyocin D (Cat. No. A-9415, Sigma) for 30 min and stimulated with 10 μM forskolin (Cat. No. 344270, Calbiochem) or 10 IU/ml ACTH (Cat. No. A-6303, Sigma) for 12 hours. Media from cultures with different treatments were collected and diluted with plain medium to fall into the standard curves for the assay. Progesterone in the cell medium was measured by using either Spectria Progesterone RIA (Cat. No. 68521, Orion Diagnostica, Espoo, Finland) or DELFIA progesterone kit (Cat. No. A066-101, PerkinElmer) following the conditions recommended by the manufacture. The cells were lysed and the StAR protein was analysed by western blotting and the level measured by densiometric analysis.

Solid phase assay and cell lysate. 5×10⁶ Y1 cells or 20×10⁶ Jurkat T cells lysed in 150 mM NaCl, 1% NP-40, 0.5% deoxycholate, 0.1% SDS and 50 mM Tris HCl (7.8) were incubated overnight at 4° C. with MEME3 and SM3 peptides synthesized on membranes (Autospot, Intavis, AG). The membranes were washed two times for 10 minutes in lysis buffer and thereafter in high salt lysis buffer (1 M NaCl) before boiling in SDS-PAGE loading buffer.

Statistics. One-way ANOVA with Tukey's post test was performed using GraphPad InStat version 3.00 (GraphPad Software, San Diego Calif. USA).

IL-2 production. T cells were cultured and activated by 10 or 50 μM Sp-8-Br-cAMPs (a cAMP agonist) for 15 minutes before the T cells were activated by anti-CD3/anti-CD28 beads (Dynal) for 20 hours. Cell free supernatants were harvested and stored at −80° C. until analsysed. The IL-2 levels were determined by Elisa (R & D Systems, Europe Ltd., UK):

Circular Dichroism. CD spectra were recorded by using a Jasco J-810 spectropolarimeter (Jasco). Measurements were performed at 25° C. by using a quartz cuvette (Starna) with a path length of 0.1 cm. All the measurements were performed with a peptide concentration of 0.10 mg/ml in PBS containing 0-75% trifluoroethanol (TFE). Samples were scanned five times at 20 nm/min with a bandwidth of 1 nm and a response time of 1 s, over the wavelength range 190 to 260 nm. The data were averaged, and the spectrum of a peptide-free control sample was subtracted. The α helical content was calculated after smoothing (means-movement method; convolution width, 13) from ellipticity data, using the neural network program CDNN version 2.1. All measurements were conducted three times.

### EXAMPLES

### Example 1

### PDSM Consensus Sequence

The alignment of sequences of the amphipathic helix motifs from mouse D-AKAP1, human AKAP149, human ezrin (unpublished), human FSC1A and human FSCIB was used to make a tentative consensus RI binding sequence. By using the MEME software (http://meme.sdsc.edu/meme/website/intro.html), an AKAP-specific position-dependent scoring matrix (PDSM) was calculated which represents the probability that an amino acid is found at a given position in the alignment divided by the frequency of this amino acid in the non-redundant protein database (FIG. 1A, lower panel). The PDSM consensus sequence developed was DELKQYANQLASQVIKEATE (20 amino acids).

The PDSM consensus sequence and the amphipathic helix motifs of mouse D-AKAP1, human AKAP149, human ezrin, human FSC1A and human FSCIB were spotted as 20-mer peptides on a membrane using a peptide array machine (Autospot, Intavis AG). They were screened for R-binding by overlay/far western by radiolabeled RI (RI mutated (A98S) protein (FIG. 1B, upper row of panel) or RII (FIG. 1B, lower rows of panel). Subsequently, binding of ³²P-labelled RI (A98S) or RII was detected by autoradiography.

The PDSM consensus sequence had a higher affinity for RI than the other R-binding motifs. Notably, when ezrin was excluded from the PDSM calculation, the consensus sequence was not able to bind RI (data not shown).

Modelling of the PDSM consensus sequence in an α-helical configuration using the helical wheel model prediction indicates that PDSM consists of one hydrophobic interface (R binding domain) and one opposite charged (acidic) and polar side (FIG. 1C). The minimal RI binding motif of the PDSM consensus sequence was found by truncations from both sides (FIG. 1D, upper panel) and offsets (FIG. 1D, lower panel). A poly-A tail was added to the C-terminus (the terminus closest to the membrane) in these experiments. Binding of RI and RII was detected both by autoradiography (³²P-labelled RI (A98S) or RII) and immunoblotting using a monoclonal antibody against RI.

A peptide sequence of 18 amino acids of PDSM (LKQYANQLASQVIKEATE) was found to have the same RI binding affinity as the full length PDSM sequence, whereas further truncation diminished the RI binding affinity. As a result the 18mer was chosen for further optimisation. The PDSM sequence and other RIα-binding peptide sequences including the RIα specific peptide, PV-38 (the most RIα specific peptide published by Burns-Hamuro et al., 2003, supra) were synthesized on a membrane (MultiPep, Intavis AG) and RIα binding was analysed by R-overlay. The PDSM sequence (LKQYANQLASQVIKEATE) exhibited a much higher RIα affinity compare to that of PV-38 (denoted with a star) (FIG. 1E).

PDSM was also shown to have higher binding to RIα compared to (Burns-Hamuro et al. 2003, PNAS 100: 4072-7) on membranes.

### Example 2

### MEME 3 Anchoring Disrupter

The PDSM sequence identified in Example 1 was further optimised. A two-dimensional array of 360 PDSM peptide derivatives was synthesized (Autospot, Intavis AG) where each residue in PDSM (given by their single-letter codes above each array) was replaced with residues having every possible side chain (given by their single-letter codes to the left of each array). The first row in each array corresponds to the native peptide (PDSM). The PDSM derivatives were analysed for R binding by either (FIG. 2A) RI-³²P- or (FIG. 2B) RII-³²P-overlay. Binding of ³²P-labelled RI (A98S) or RII was detected by autoradiography.

Substitutions of the polar amino acid at position 4 or hydrophobic amino acids at positions 5 or 8, 9, 12, 13 and 16 (solid line boxed columns on FIGS. 2 A and B), generally decreased both RI and RII binding. Importantly, substitutions with aspartic acid (D) or glutamic acid (E) (dotted columns) in most of the positions in between these columns (at position 1, 2, 3, 6, 7, 10, 11, 14, 15, 17 and 18) were well accepted for RI binding but were in most cases as bad as proline substitution for RII-binding (dotted rows in FIGS. 2A and B).

PDSM derivatives with higher RI affinity and lower RII affinity or higher affinity for both RI and RII are indicated by black circles and squares, respectively. White circles denote peptides in the array that corresponds to the native PDSM sequence.

Almost 150 selected different modified PDSM derivatives (single, double, triple and quadruple substitutions) were synthesized on membranes (Autospot, Intavis AG) and R-binding analysed by RI-³²P- and RII-³²P-overlays. Certain of these are shown in FIG. 2C. The relative RI and RII binding affinities were measured by densitometry analysis of the autoradiographs (n=3). The RI-³²P- (upper panel) and RII-³²P (middle and lower panel, for different exposures). Binding of the ten PDSM derivatives with the highest RI affinity are shown.

The peptide with the triple mutation, K2E, S10D, V12I (MEME3) had a 1.9 times higher affinity for RI compared to that of PDSM and, in addition, a 0.5 times lower affinity for RII (FIG. 2C lower panel of RII overlay), and was the peptide with the most apparent RI selective profile.

Modelling of the RI specific sequence in an α-helical structure using the helical wheel model shows that MEME3 consists of one hydrophobic interface (R binding domain) and one opposite charged (acidic) and polar side. The dotted circles represent substitutions versus PDSM. Valine (V) was substituted with isoleucine (I) at the hydrophobic side, whereas lysine (K) and serine (S) were substituted with aspartic acid (D) and glutamic acid (E), thus making MEME3 even more acidic than PDSM (FIG. 2D).

### Example 3

### In Vitro Characterization of MEME3 Binding properties

Dissociation constants (K_{D}) for MEME3 and RI (bovine) (FIG. 3A) or RII (mouse) (FIG. 3B) were measured by fluorescence polarization. RIα bound much more tightly with a K_{D} of 0.82+/−0.22 nM (n=5), whereas the K_{D} for RIIα was 1.6+/−0.22 μM (n=3). A control peptide of identical amino acid composition but with a scrambled sequence did not interact with either R subunit. The K_{D} for the N-truncated RIα(Δ1-11) was 0.78+/−0.27 nM and in similar range as full length RIα (not shown).

MEME3 selectivity for RI over RII is shown (FIG. 3C). MEME3 has an approximately 2000 times higher affinity for RI than for RII.

The binding of MEME3 to human RI was also demonstrated. The MEME3 sequence was synthesized on a membrane and RIα binding analysed by R-overlay using equal amounts of either human or bovine RIα (FIG. 3C). Human RIα bound with similar affinity to MEME3 as did bovine RIα (FIG. 3D, n=3). The negative control peptide, SM3, exhibited no RIα binding at all.

### Example 4

### Interaction of MEME3 with PKA Type I Inside Cells

GFP-fusion proteins with the MEME3 (FIG. 4A upper) and scrambled MEME3 (FIG. 4A lower) peptides used in the cell culture studies were generated.

HEK293 cells were transfected with either construct. RI (FIG. 4B), RII (FIG. 4C) or GFP (FIG. 4D) were immunoprecipitated using monoclonal or polyclonal antibodies, respectively.

Immunocomplexes were analysed on 10% PAGE (B and C) or 15% PAGE (D) and subjected to Western blotting using anti-GFP, anti-RI or anti-RII as indicated and blotted onto PVDF membranes. Co-precipitation of MEME3 with RI (lane 2 in FIG. 4B) but not with RII (lane 2 in FIG. 4C) was detected by immunoblotting using polyclonal antibodies against GFP. Cells transfected with GFP alone were used as a negative control (lane 1).

In FIG. 4D, the co-precipitation of recombinant full length and N-truncated RI (added to the cell lysate) with MEME3 was detected by immunoblotting using a monoclonal antibody against RI (lane 2-4). The RI(Δ1-24) protein which is not able to dimerize did not precipitate MEME3 and was used as negative control (lane 5).

Scrambled MEME3 (SM3) did not co-precipitate with RI (lane 6). The level of endogenous RI was probably too low to detect (lane 1).

### Example 5

### Characterization of the Extent of the PKA Type I Binding Site by Proline Scanning Mutations

Native MEME3 and peptides with a proline substitution in each position from 1 to 18 were synthesized on a membrane (Autospot, Intavis AG) and RI binding was analysed by RI-³²P-overlay. Proline substitutions in the MEME3 sequence strongly reduced (positions 2, 3, 15 and 18) or disrupted (positions 1, 3-14, 16 and 17) the RI binding, indicating that each position is important for binding. The C-terminus appeared less important than N-terminus for MEME3 binding to RI as indicated by the weak signals of R-binding of the peptide with the E18P substitution.

Specificity of MEME3 interaction with PKA type I was determined by competition experiments. 500 nM MEME3 peptide in solution competed the RI-binding (RI-³²P-overlay) to MEME3 synthesized on membrane (Autospot, Intavis AG) (FIG. 2B), whereas up to 100 μM of MEME3 peptide was not able to compete the RII binding (RII-³²P-overlay) to an RII specific binding sequence.

### Example 6

### MEME3 Disrupts PKA Type I Anchoring in T Cells

Membrane microdomains in the cell membrane (lipid rafts) that form contacts with target cells (immunological synapses) containing the signalling machinery were purified from Jurkat T cells transfected with MEME3 and Scrambled MEME3 (SM3) (FIG. 6A). The PKA content was measured by densitometry analysis (n=1) and showed that RI concentration in cells transfected with MEME3 was reduced by 400 compared to cells transfected with SM3 (FIG. 6B). The observed reduction of 400 is consistent with the transfection efficiency.

Jurkat T cells were incubated with MEME3-Arg₁₁ peptide at different concentrations. All membrane localized RI was displaced and was delocalised at 5 μM MEME3-Arg₁₁, whereas some displacement of RI from cell membrane was observed at 1 μM MEME3-Arg₁₁. Arginine-rich peptides have been reported to be able to enter cells with high efficacy.

### Example 7

### Additional RI Anchoring Disruptors

78 PDSM derivatives had a higher RI specificity than PDSM (FIG. 7A-E).

In addition, 28 peptides with C-terminal truncations (16 or 17 amino acids in length) had a higher RI specificity than PDSM (18aa).

Altogether a set of 122 PDSM derivatives (16-18 aa) were developed with a higher RI specificity than PDSM.

In an additional two-dimensional peptide array, 20 MEME3 derivatives with a higher RI specificity than MEME3 were discovered (FIG. 7G).

The derivatives contain the additional single substitution; L1C, E2D, Q3D, Q3E, N6D, N6E, Q7D, Q7E, D10G, Q11D, Q11E, Q11I, K14A, K14D, K14E, K14M, K14R, K14T, K14W or K₁₄Y.

### Example 8

### MEME3 Disrupts PKA Type I Anchoring in Isolated Human T Cells

### Methods

Isolation of T cells. Human peripheral blood T cells were purified by negative selection (95-98% pure) as described (Aandahl E M et al., 1998, FASEB J. 12:855-62). Lipid Raft Purification. Isolation of lipid rafts or glycosphingolipid-enriched membrane microdomains from T cells was performed by small scale ultracentrifugation. In brief, 20 million cells were homogenized in 250 μl ice-cold lysis buffer (50 mM Hepes, pH 7.4, 100 mM NaCl, 5 mM EDTA, 0.70 Triton X-100, 10 mM sodium pyrophosphate, 1 mM Na₃VO₄, 50 mM NaF, 1 mM PMSF, and protease inhibitors) by 10 pestle strokes in a Dounce homogenizer, loaded at the bottom of a 40 to 50 sucrose gradient and centrifuged at 200,000 g for 5 h. 80 μl fractions were collected from the top. The fractions were analysed by immunoblot analysis. LAT was used as a marker for lipid raft fractions.

IL-2 production Assay. Primary T cells were stimulated or not with anti-CD3/anti-CD28 coated beads (Dynal, cat. no. 111.31) for 20 hours, thereafter supernatants were harvested and the concentration of IL-2 was assessed by ELISA (R&D Systems, cat. no D2050). When used, 8-CPT-cAMP was added for 15 minutes before CD3/CD28 treatment.

Membrane microdomains in the cell membrane (lipid rafts) that form contacts with target cells (immunological synapses) containing the signaling machinery were purified from primary human T cells incubated with 10 μM MEME3-Arg₁₁ or 10 μM Scrambled MEME3-Arg₁₁ (SM3-Arg₁₁) peptide by sucrose gradient centrifugation. Fractions corresponding to lipid rafts and non-raft fractions were subjected to immunoblot analysis with the indicated antibodies (FIG. 8A). The PKA content was measured by densitometry analysis (n=4) and showed that RI and C concentration in cells incubated with MEME3 was reduced by 80% compared to cells loaded with SM3 (FIG. 8B). No PKA-RII was observed in the lipid rafts fractions (not shown). Arginine-rich peptides have been reported to be able to enter cells with high efficacy.

T cells were incubated with MEME3-Arg₁₁ peptide at different concentrations. Displacement of RI from T cell membranes was observed at 5 μM MEME3-Arg₁₁, whereas no displacement of RI was seen at 5 μM of the scrambled MEME3-Arg₁₁ peptide (FIG. 8C). T cells have more RI compared to Jurkat T cells. A 2.5 fold increase in IL-2 production was seen for T cells incubated with 10 μM MEME3-Arg₁₁ (FIG. 8D). Levels of IL-2 secretion are presented relative to the level in untreated T cells.

### Example 9

### Identification of Amino Acids in MEME3 Important for RI Affinity and Specificity

The native MEME3 sequence (18-mer) and peptides with an alanine, aspartic acid, lysine, serine or proline in each position from 1 to 18 in the sequence were synthesized on a membrane (MultiPep, Intavis AG) and RIα and RIIα binding were analysed by overlay. The hydrophobic interface appeared to be unchangeable. In addition, Glu2, Tyr4 and Lys14 appeared to be important for RIα binding and any substitution of these amino acids abolished the RIα binding. Some amino acids outside the hydrophobic interface were important for the RIα specificity, and substitutions of N6, D10 and E1S greatly reduced the RIα specificity.

### Example 10

### Identification of the MEME3 Binding Site in the N-Terminus of RIα

MEME3 and SM3 (scrambled negative control peptide) were cloned by preferred codon usage (Haas et al., 1996, Current Biol., 6:315-324) and with an N-terminal GFP-fusion. Lysate from transfected HEK293 cells (FIG. 10, left panel in A) and MEME3 synthesized on membrane (right panel in A) were analysed for R binding by RIα-³²P-overlay and RIIα-³²P overlay. Both GFP-MEME3 (left panel, lane 2) and MEME3 on membrane (right panel) bound with high affinity to RIα, and with even higher affinity to the N-terminal RIα deletion mutants, RIα (Δ1-11) and RIα (Δ1-15). No binding to RIα (Δ1-24) which had lost AKAP binding and dimerization determinants was observed. Thus, the most important MEME3 binding determinants reside within amino acids 16-24 of the RIα dimer. No R-binding was observed to GFP (lane 1, left panel) or the negative control protein, GFP-SM3 (lane 3).

Dissociation constants (K_{d}) for the interaction of N-terminal RIα deletion mutants with MEME3 measured by fluorescence polarization (FIG. 10, B) were 0.82+/−0.10 nM for RIα wildtype, 0.78+/−0.13 nM for RIα (Δ1-11) and 0.45+/−0.09 nM for RIα (Δ1-15).

Altogether, the N-terminal RIα deletion mutants bound more tightly to MEME3. The binding surface might be more accessible in the N-terminal RIα deletion mutants. No RIα binding of the negative control peptide, SM3, was observed.

### Example 11

### MEME3 Co-Precipitates Exclusively Endogenous PKA Type I from T Cell Lysate

Solid phase pull down (see schematic illustration in FIG. 24A, left panel) was performed to analyse endogenous PKA binding to MEME3. MEME3 and SM3 peptides synthesized in triplicate on membranes were incubated overnight in T cell lysate, thereafter bound proteins were eluted, subjected to 10% PAGE electrophoresis and analysed by immunoblotting (FIG. 11). The presence of PKA subunits was assessed by immunoblotting using specific antibodies against PKA-RIα and PKA-RIIα subunits. PKA-RIα, but not PKA-RIIα, was immobilized by MEME3. Neither PKA-RIα nor PKA-RIIα bound to the negative control peptide, SM3.

### Example 12

### MEME3 Co-Precipitates Endogenous PKA Kinase Activity

The specific activity (pmol/min per IP) of PKA-C subunit from HEK293 cells co-precipitating with chimeric MEME3 fusion protein was measured by a phosphotransferase assay using Kemptide as a substrate (Kemp et al., J. Biol. Chem. 252, p 4888-4894, 1977; Roskoski, R. Methods Enzymol. 99, p 3-6, 1983) in the absence and presence of cAMP (FIG. 12). Specific PKA activity was abrogated when PKI (5-24) peptide (10 μM) was added to the reaction mixture. The MEME3 fusion protein coprecipitated 17-fold more PKA-C activity than the RIIα-selective peptide, AKAP-is (IS) (Alto et al., 2003, supra). No kinase activity was detected in the negative controls (scrambled AKAP-is (S-IS), S-MEME3 (SM3) or IgG). The accumulated data from three independent experiments are shown.

### Example 13

### Toxicity of MEME3-Arg₁₁ in Cell Based Assays

To analyse the toxicity of the MEME3-Arg₁₁ peptide, Jurkat T cells were treated with different concentrations of MEME3-Arg₁₁, incubated for different periods of time and examined for viability by propidium iodide (PI) exclusion assay. No toxicity was observed after 4.5 hours, 1 or 2 day incubation with 25 μM MEME3-Arg₁₁. SM3 and Arg₁₁ were used as negative control peptides. Anisomycin treated Jurkat T cells were used as a positive control.

### Example 14

### Effect of MEME3-Arg₁₁ on Cell Death

To test whether MEME3-Arg₁₁ induces cell death or apoptosis in human peripheral blood T cells, activated T cells treated with increasing doses of MEME3-Arg₁₁ were analysed by forward scatter/sideward scatter (FSC/SSC) in a fluorescence-activated cell sorter (FACS). Activated T cells incubated with increasing doses of MEME3-Arg₁₁ showed no morphological changes characteristic of apoptosis (two upper most panels) with decreased FSC and increased SSC. Apoptosis was also assessed by Annexin V^{FITC} labeling under the same experimental conditions. MEME3-Arg₁₁ treated cells showed no increase in annexin V binding, but rather a small decrease suggesting a small rescue effect by the M3-Arg₁₁ peptide (FIG. 14, second from bottom). The effect of the peptides on cellular DNA content was also measured by flow cytometry after permeabilization of the fixed cells and PI-staining. No change in DNA content (aneuploidity) was observed for T cells incubated with M3-Arg₁₁ (bottom).

### Example 15

### Levels of Late Apoptotic (AV+/PI+) T Cells Following Treatment with MEME3-Arg₁₁

Cell death was also analysed by monitoring PS externalization by Annexin V binding and destruction of the cellular membrane by PI staining using FACS. No late apoptotic cells (AV+/PI+) were detected in samples treated with MEME3-Arg₁₁ or with the negative control peptide SM3-Arg₁₁ overnight (FIG. 15).

### Example 16

### MEME3-Arg₁₁ Disrupts PKA Type I Anchoring from Cell Membranes in Human Primary T Cells

Isolated human peripheral blood T cells were treated with MEME3-Arg₁₁ peptide at different concentrations, and the location of PKARIα determined by immunofluorescence. All PKA-RIα that localized at the cell periphery (pericortical region) in the absence of added peptide was displaced and delocalized by 5 μM MEME3-Arg₁₁ (FIG. 16, left panel, left column).

In contrast no displacement of PKA-RIIα (stained using an anti RII antibody) from centrosomes (stained using an antibody to AKAP450) was observed in MEME3-Arg₁₁ treated cells or cells treated with the negative control peptide, SM3-Arg₁₁ (FIG. 16, left panel, right column). The RIIα specific peptide, Arg₁₁-super-AKAP-is, specifically displaced PKA-RIIα from anchored sites in centrosomes and was used as a positive control for RIIα anchoring disruption (FIG. 16 right panels).

### Example 17

### MEME3-Arg₁₁ does not Disrupt Anchoring of Particulate PKA Type II in T Cells

To assess the effect of MEME3-Arg₁₁ on anchoring of PKA type II, T cells were treated with MEME3-Arg₁₁ or Arg₁₁-super-AKAP-is (as a positive control) and particulate fractions were isolated, followed by 10% PAGE and blotting onto a PVDF membrane (fraction no. 2-4, FIG. 17 in upper panel) and analysed by immunoblotting using antibodies against PKA-RIIα (FIG. 17, upper panel). The levels of PKA-RIIα in the particulate fractions were measured by densitometry of the autoradiograms (lower panel). The level of PKA-RIIα was set relative to that of the internal standard PKCα. No reduction in PKA-RIIα was observed with increasing concentrations of MEME3-Arg₁₁. In contrast, approximately 80% reduction of PKA-RIIα was observed in T cells treated with 25 μM or 50 μM of the RIIα-specific peptide, Arg₁₁-super-AKAP-is.

The MEME3 peptide is therefore specific to RIα as it does not disrupt anchoring of particulate PKA type II in T cells.

Similar specificity of the peptide inhibitor for the PKA I pathway was shown using whole cell patch-clamping techniques to observe time-dependent down-regulation of α-amino-3-hydroxy-5-methyl-4-isoxazolepropionic acid.

### Example 18

### MEME3-Arg₁₁ Disrupts PKA Type I Anchoring in Lipid Rafts of Human Peripheral Blood T Cells

Lipid rafts were purified from isolated human peripheral blood T cells treated with MEME3-Arg₁₁ or SM3-Arg₁₁ for 12 hours, followed by 10% PAGE and blotting onto a PVDF membrane and immunoblotting (FIG. 18, left panel). The PKA content was measured by densitometry of immunoblots (n=4). PKA type I in cells treated with 10 μM or 25 μM M3-Arg₁₁ was reduced by 80% and 90%, respectively. The very weak signal observed after immunoblotting using antibodies against RIIα (data not shown) was measured by densitometry of the autoradiogram. The PKA-type II level was not reduced in lipid rafts from human peripheral blood T cells treated with MEME3-Arg₁₁ (FIG. 18, right panel, n=4).

### Example 19

### MEME3-Arg₁₁ Inhibits PKA Phosphorylation of Csk in Forskolin-Stimulated Human Peripheral Blood T Cells

Peripheral blood T cells were treated with 25 μM M3-Arg₁₁ or mock treated for 12 hours and stimulated with forskolin to activate the PKA-Csk inhibitory pathway. Subsequently, cell lysates were subjected to 100 PAGE, blotting onto a PVDF membranes and the Csk phosphoserine 364 levels were assessed by immunoblotting using phosphospecific antibodies to Csk-PS364 (FIG. 19, upper panel). Csk-PS364 levels were next measured by densitometry revealing that the Csk-PS364 level was reduced by 70% in T cells treated with 25 μM M3-Arg₁₁ (FIG. 19, lower panel).

### Example 20

### MEME3-Arg₁₁ Inhibits Csk Phosphorylation of the C-Terminal Tyrosine in Lck (Lck-.Y505) in Forskolin-Stimulated Human Peripheral Blood T Cells

Peripheral blood T cells were incubated in the absence (FIG. 20, A) or presence of 25 μM MEME3-Arg₁₁ (B) or 25 μM SM3-Arg₁₁ (C) for 12 hours and stimulated with forskolin to activate the PKA-Csk inhibitory pathway. Subsequently, lipid rafts were purified by sucrose gradient centrifugation and fractionation. Next, fractions were analysed by 10% PAGE, blotting onto a PVDF membrane and immunoblotting using phosphospecific antibodies to Lck-PY505 (FIG. 20, panels A-C). Lck-PY505 content was measured by densiometry (n=3) (FIG. 20D) indicating that the LckPY505 levels in cells treated with 25 μM MEME3-Arg₁₁ were reduced by 60% compared to that of SM3-Arg₁₁ treated cells.

### Example 21

### MEME3-Arg₁₁ Reverses CAMP Inhibition of IL-2 Production in Activated Human Peripheral Blood T Cells

To examine the effect of MEME3 on cytokine production, IL-2 levels in medium from human peripheral blood T cells treated with MEME3-Arg₁₁ were measured by ELISA. First, cells were treated with MEME3-Arg₁₁ for 12 hours, thereafter the PKA inhibitory pathway was activated by 10 or 50 μM Sp-8-Br-cAMPs (a cAMP agonist) for 15 minutes before the T cells were activated by CD3/CD28 stimulation (FIG. 21). As seen from the figure, increasing concentrations of cAMP inhibited CD3/CD28 stimulated IL-2 levels. Furthermore, the levels of IL-2 were increased in samples treated with MEME3-Arg₁₁ in a concentration-dependent manner. Cells treated with 25 μM MEME3-Arg₁₁ and 10 μM Sp-8-Br-cAMPs had IL-2 production levels similar to that of activated T cells without cAMP. The positive effect of re-loading of the peptide was also demonstrated (10+10 μM MEME3-Arg₁₁): Cells which were reloaded with 10 μM MEME3-Arg₁₁ (before the Sp-8-Br-cAMPs treatment) had an even higher IL-2 production.

Hyperactivated PKA-signaling leads to T cell dysfunction in HIV (as illustrated in FIG. 22, upper panel), whereas anchoring disruption by MEME3 restores T cell function (lower panel).

### Example 22

### MEME3-Arg₁₁ Increases Immune Function in Mice with Murine AIDS

Rad-LS infected mice with MAIDS (10-12 weeks postinfection) were treated with MEME3-Arg₁₁ in doses corresponding to those used for other bioactive peptides in studies in mice. Parenteral administration was accomplished by injecting intraperitoneally peptide formulated in PBS. Thus, MAIDS mice were injected with vehicle (phosphate buffered saline, PBS, Ctr.) or treated with MEME3-Arg₁₁ in PBS (M3) by intraperitoneal injection (75 mg/kg/day administered once daily) for 10 days (left panel). In parallel, healthy, uninfected mice were injected with PBS (Ctr.) or MEME3-Arg₁₁ in PBS (M3) (right panel). Subsequently, T cell proliferative responses were assessed in vitro in a mixed population of unsorted lymph node mononuclear cells from treated and untreated animals by [³H]-thymidine incorporation. T cell activation was accomplished in all samples by cross-ligation of anti-CD3 (mAb 2C11; 4 g/ml). Cells were cultured for 72 h during which MEME3-Arg₁₁ was added back. [³H]-thymidine was included for the last 4 hours. When T cell immune function was assessed in crude lymph node cells from infected and healthy treated and untreated mice after 10 days of injection, it was clear that whereas untreated infected animals had anti-CD3 induced proliferation in the range of 50000 cpm independent of whether or not peptide was injected, infected mice that received MEME3-Arg₁₁ for 10 days had T cell responses to anti-CD3 that were increased more than three-fold compared to infected, control-treated mice (FIG. 23, left panel). In contrast, healthy mice had similar immune responses independent of whether MEME3-Arg₁₁ peptide was injected or not (FIG. 23, right panel).

### Example 23

### MEME3 Immobilizes Endogenous PKA Type I but not PKA Type II from Y-1 Adrenal Cell Lysate

A solid phase immobilization assay (schematic illustration in FIG. 24, left panel) was used to analyse endogenous PKA binding to MEME3. MEME3 and SM3 peptides synthesized in triplicate on membranes were incubated overnight in lysate from Y-1 adrenal cells. Thereafter bound proteins were eluted, subjected to 10% PAGE and analysed by immunoblotting using specific antibodies against PKA-RIα, PKA-C and PKA-RIIα subunits. Only PKA type I was immobilized by MEME3. Neither PKA-RIα, PKA-C nor PKA-RIIα were immobilized by the negative control peptide, SM3.

### Example 24

### MEME3-Arg₁₁ Disrupts PKA Type I Anchoring at Mitochondria in Mouse Y-1 Adrenal Cells

In Y1 adrenal cells loaded with MEME3-Arg₁₁ endogenous PKA-RIα was displaced from mitochondria (FIG. 25, upper panels) as evident from immunostaining where localization of RIα at mitochondria was indicated by yellow in the image overlays of RIα (green) and mitotracker (red) and displacement was indicated by separation of green and red. In contrast, the subcellular distribution of RIα was not affected by loading of cells with SM3-Arg₁₁ (FIG. 25, lower panels). Thus, MEME3-Arg₁₁ disrupts PKA type I anchoring to mitochondria in Y1 adrenal cells.

### Example 25

### MEME3-Arg₁₁ Inhibits Progesterone Synthesis in Y1 Adrenal Cells

Hormone-stimulated progesterone production induced by ACTH or forskolin and measured by RIA assay was reduced by approximately 40% to 50% in cells treated with MEME3-Arg₁₁ compared to non treated cells (FIG. 26, P<0.001).

The effect of MEME3-Arg₁₁ on PKA phosphorylation of StAR was analysed by measuring the progesterone production in Actinomycin D treated cells (this treatment blocks StAR synthesis). A short pre-stimulation with forskolin was applied to increase the basal level of StAR before further synthesis was blocked with Actinomycin D and cells were re-stimulated with forskolin. The progesterone production was reduced by 60% in Actinomycin D treated cells incubated with MEME3-Arg₁₁ compared to the negative control peptide, SM3-Arg₁₁ (P<0.05). The inhibition was approximately 80% of the inhibition that was observed when the PKA phosphorylation site in StAR was mutated (Arakane et al., 1997, J Biol Chem, 32: 32656-32662).

### Example 27

### MEME3-Arg₁₁ Reduces Steroidogenic Acute Regulatory Protein (StAR) Synthesis

StAR protein levels in the samples from Example 28 were analysed by immunoblotting. StAR consists of a 37-kDa precursor form containing an N-terminal mitochondrial targeting sequence and a processed 30-kDa mature protein (Epstein and Ormejohnson, 1991; Stocco and Sodeman, 1991). Y1 adrenal cells expressed a low basal level of StAR (FIG. 27, upper panel, lane 1). Upon forskolin stimulation, a significant increase in mature StAR protein (lane 2) occurred consistent with an increase in cholesterol transport over the mitochondrial membrane and a corresponding increase in steroid biosynthesis (bar diagram in FIG. 26). The forskolin-stimulated levels of StAR protein were reduced by 60% in MEME3-Arg₁₁ treated cells (lane 5 and lower panel). In contrast, treatment with SM3-Arg₁₁ reduced StAR levels by less than 20% (lane 8, and lower panel). Thus, MEME3-Arg₁₁ reduced StAR levels by twice as much as SM3-Arg₁₁. In Actinomycin D treated cells, the StAR level was similar to the basal levels confirming that StAR synthesis was blocked (lane 3, 6 and 9). It is noteworthy that a somewhat higher basal StAR level was observed in cells treated with MEME3-Arg₁₁ or SM3-Arg₁₁ (lane 4 and 7). The level of the 37-kDa precursor form was unchanged in all samples (data not shown).

### Example 28

### MEME3-Arg₁₁ Inhibits PKA Type I Signaling Pathway and Progesterone Production in Steroid Producing Cells

A relevant model for studying the biological effect of MEME3 involves analyzing the effect of anchoring disruption of PKA type I on cAMP-regulated steroidogenesis. The cAMP-PKA signaling pathway is involved in regulation of steroid biosynthesis at several levels. Hereunder PKA phosphorylates the transcription factor (CREB) regulating steroidogenesis acute regulatory protein (StAR) gene expression resulting in an increased level of StAR protein upon ACTH stimulation (Reinhart et al., 1999, Mol Cell Endocrin., 151: 161-169). Thereafter, PKA phosphorylates and activates the newly synthesized StAR protein, which then accelerates the transport of cholesterol, substrate for steroid synthesis, into mitochondria where cholesterol serves as substrate for p450scc, the side chain cleavage enzyme that converts cholesterol into pregenolone in the first, rate limiting step in steroid biosynthesis. PKA phosphorylation of StAR is essential for conferring the biological function of this protein and for steroid biosynthesis (Jo et al., 2005, Biol. Reprod, 73: 244-255). PAP7 has a central role by anchoring PKA-RIα in association with the peripheral-type benzodiazepine receptor (PBR) at the mitochondria to regulate cholesterol transport (Li et al., 2001, Mol. Endocrin., 15: 2211-2228; Liu et al., 2003 supra).

The involvement of the PKA type I signaling pathway and effect of anchoring disruption in the regulation of StAR phosphorylation and steroidgenesis is summarized in FIG. 28. In the absence of hormonal stimuli (FIG. 28, A), the StAR level is very low and no cholesterol is transported into the mitochondria. PKA type I is targeted to mitochondria through binding to PAP7 which is associated with peripheral-type benzodiazepine receptor (PBR) (Liu et al., 2003, supra). Upon hormonal stimulation (FIG. 28, B), cAMP binds anchored PKA type I which results in release of active catalytic subunit and phosphorylation of the newly synthesized StAR protein leading to activation of the steroidgeneic activity of the protein. PBR is organized in clusters and forms a multimeric pore complex with the 34-kDa voltage-dependent anion channel (VDAC), thus allowing the translocation of cholesterol to the inner membrane. The side-chain cleavage cytochrome, P450ssc, is the first enzyme of the steroidgenic pathway and responsible for transformation of cholesterol into pregnenolone, which is further transformed into progesterone. The polypeptide diazepam binding inhibitor (DBI) is an endogenous PBR ligand which stimulates the cholesterol transport. Upon anchoring disruption (FIG. 28, C), the PKA-PAP7 interaction is disrupted, the StAR protein is not phosphorylated upon hormonal stimulation. Thus, the steroidgenic activity of StAR as well as the steroid biosynthesis is not activated.

### Example 29

### MEME3 Peptidomimetics and R-Binding

To make a more stable PKA type I anchoring disruptor, different MEME3 and MEME3-Arg₁₁ derivatives (peptidomimetics) with D-amino acid substitutions (in small letters) were synthesized on membrane and RIα and RIIα-binding analyzed by R-overlay. D-amino acid substitutions were accepted in position 18 (E18e) (circled in FIG. 29A). D-amino acid substitutions in the Arg₁₁-tail of MEME3-Arg₁₁ or outside the core MEME3 sequence were in general accepted (circled in FIG. 29B).

SM3-Arg₁₁ (in FIG. 29B) or SM3 (in FIG. 29C) were used as negative controls as non-R-binding sequences, whereas the RIIα specific sequence of Arg₁₁-super-AKAP-is was used as a positive control for RIIα binding (in FIGS. 29, B and C). D-amino acid substitutions outside the MEME3 core sequence were well accepted. Especially, the D-amino acids of arginine and leucine (circled in FIG. 29C) in e.g. l-MEME3 and MEME3-r which exhibited similar RIα affinities and RIα specificities as the regular MEME3 peptide (on the top in FIG. 29C) were well accepted. D-amino acid substitutions of arginine and leucine were also well accepted at both the N- and C-termini of MEME3. The R-binding of the different combinations thereof are shown in FIG. 29D.

### Example 30

### Stability of MEME3 and Derived Peptidomimetics in Human Serum

The stability of a selection of PKA-AKAP anchoring disruptors was analyzed. M3 (LEQYANQLADQIIKEATE), M3Hrr (LEQYANQLADQIIKEATEHrr), M3RRR (LEQYANQLADQIIKEATERRR), M3(E18) (LEQYANQLADQIIKEATe), and M3-Arg11 (LEQYANQLADQIIKEATERRRRRRRRRrr) were diluted in 10% human serum (not heat-inactivated) at a concentration of 300 μM and incubated at 37° C. for a total of 48 hours. Sample aliquots were withdrawn at defined time intervals, whereupon enzymatic activity was terminated with the addition of 7.5° (vol/vol) trifluoroacetic acid (TFA). The full-length peptide content of each sample was determined with UV-detection at 220 nm following reversed phase high-performance liquid chromatography with gradient elution over a C18 aliphatic column. Relative levels of peptide as a function of time of incubation was plotted (FIG. 30) and half-lives were calculated. While the half lives of MEME3-derived peptidomimetics appeared shorter than that of MEME3, the MEME3 sequence itself was protected in several of these peptides such that the core MEME3 sequence itself actually obtained a longer half life (e.g. in the MEME3-Arg₁₁ configuration).

### Example 31

### Enhanced PKA Type I Signaling by Cellular MEME3 Targeting The high affinity PKA type I binding sequence, MEME3, was coupled to the DRM targeting domain of Lck (1-14 aa), two glycine sequences of ten glycine residues each were introduced as spacers between the two functional domains and a myc tag (although any tag may be used) was added in between (FIG. 31, A). Lipid rafts were isolated from Jurkat T cells transfected with this construct (called Lck-M3), followed by separation by 4-20% PAGE, blotting onto a PVDF membrane and analysis of levels of Myc, PKA-C, PKA-RIα, LckPY505 and LAT by immunoblotting (FIG. 31, B). Wildtype Jurkat T cells were used as a negative control. The relative levels of PKA-C, PKA-RIα, LckPY505 and LAT were measured by densitometry of the autoradiograms (FIGS. 31, C and D). LAT was used as a marker for lipid rafts and was used as an internal standard. 2- and 7-fold increases in the levels of PKA-C and PKA-RIα, respectively, were observed for Jurkat T cells transfected with Lck-M3 (FIG. 31, C) indicating the capacity of the Lck-M3 to redistribute PKA type I. The level of Lck-PY505 in Lck-M3 transfected cells was higher in the basal state (25% higher) as well as in forskolin stimulated cells (63% higher) compared to that of wildtype (FIG. 31, D).

### Example 32

### MEME3 Targeted to Lipid Rafts Enhances PKA Type I Signaling and Inhibits T Cell Function

FIG. 32 depicts how MEME3 attached to lipid rafts through the DRM targeting motif of Lck recruits additional PKA type I to the lipid rafts which increases PKA phosphorylation and activation of Csk, resulting in increased C-terminal phosphorylation of Lck and inhibition of T cell function.

### Example 33

### Further Optimisation of MEME3

The MEME3 sequence identified in Example 2 was further optimised. A two-dimensional array of 360 MEME3 peptide derivatives was synthesized (Multipep automated peptide synthesizer, INTAVIS Bioanalytical Instruments AG, Koeln, Germany) where each residue in MEME3 (given by their single-letter codes above each array) was replaced with residues having every possible side chain (given by their single-letter codes to the left of each array). The first row in each array corresponds to the native peptide (MEME3). The MEME3 derivatives were analyzed for R binding by either RIα-³²P- (FIG. 33, A) or RIIα³²P-overlay (FIG. 33, B). Binding of ³²P-labeled RIα (A98S) or RIIα was detected by autoradiography. The position of hydrophobic amino acids at positions 1, 5, 8, 9, 12, 13 and 16 are in boxes.

MEME3 derivatives with higher RIα affinity and lower RIIα affinity are indicated by circles. These derivates contain the additional single substitution: L1F, L1I, L1Y, Q3A, Q3D, Q3E, Q3K, Q3M, Q3R, Q3S, Q3T, N6G, N6T, Q7K, Q7L, Q7M, Q7R, Q7S, Q7T, D10G, D10N, Q11K, Q11L, Q11R, K14R, T17C, T17M, E18M, E18N, E18Q and E18T. White circles denote peptides in the array that correspond to the native MEME3 sequence.

### Example 34

### Additional RI Anchoring Disruptors

Modified MEME3 derivatives (single and double substitutions) with a higher RIα specificity than MEME3 (up to almost a 70-fold increase in RIα specificity) are shown in FIG. 34 A-C.

### Example 35

### Preferable Position for Substitutions in MEME3

The most preferable positions for making substitutions and the most preferred substitutions are marked in the MEME3 sequence and in the helical wheel model of the α-helical structure of MEME3 (FIG. 35 with arrows). These are Q3A, Q3M, Q3E, Q3S, Q3K, Q3R, Q7R, Q7K, Q7M, Q7L, Q11K, Q11R, E18Q, E18T and E18M.

### Example 36

### Helical Content of MEME3

Modeling the MEME3 sequence into an α-helical wheel showed that MEME3 consists of a hydrophobic side (R binding interface) and an opposite charged and polar side. A control peptide of identical amino acid composition but with a scrambled sequence (scrambled MEME3=SM3) containing no amphipathic helix motif was used as a negative. The helicity of MEME3 and SM3 measured by circular dichroism were 49% and 29% in 50% TFE, respectively. In the absence of TFE, the peptides maintained a conformation that included 12% and 7% helicity, respectively. The helicity of the MEME3-Arg₁₁ peptide was 39% in 50% TFE.

1. A PKA I anchoring disrupting molecule or AKAP mimic, wherein said molecule or mimic is a polypeptide which comprises the following amino acid sequence:
X₁ X₂ X₃ Y A X₄ X₅ L A X₆ X₇X₈ I X₉ X₁₀ X₁₁ X₁₂ X₁₃ (sequence (1)) wherein
X₁ is L, C, I, Y, V, W or F;
X₂ is K, R, H, E, D, C, V, A, I, Q, S, T or L;
X₃ is Q, D, E, A, S, I, F, K, R, L, M, T, G, N, W or V;
X₄ is N, D, E, S, A, M, K, R, G, T, W or Q;
X₅ is Q, D, E, M, F, I, S, K, R, C, W, Y, L or T;
X₆ is S, D, M, N, E, I, A, R, F, H, W, K, L, Y, Q or G;
X₇ is Q, D, E, I, K, R, T, V, F, N, S, L, W or M;
X₈ is I, A, S, L, D, E or V;
X₉ is K, C, D, E, R, A, M, T, W, H, Q, Y, L or S;
X₁₀ is E, D, R, Q or K;
X₁₁ is A, C, I, F, L, G, H or V;
X₁₂ is T, C, L, F, I, V, M, K, R or W; and
X₁₃ is E, D, N, V, Y, K, A, F, G, H, I, Q, L, M, R, S, T or W,
or a peptidomimetic or analogue thereof.

2. The anchoring disrupting molecule or AKAP mimic of claim 1 wherein
X₁ is L, C, I, or F;
X₂ is K, R, D, or E;
X₃ is Q, D, E, A, S, I, V;
X₄ is N, D, E, or S;
X₅ is Q, D, E, F, I, M;
X₆ is S, M, N, E, D, L or T;
X₇ is Q, M, E, or D
X₈ is I, A, S, L, D, E or V;
X₉ is K or R;
X₁₀ is E or D;
X₁₁ is A;
X₁₂ is T, L or W; and/OR
X₁₃ is E, D, R, K or W.

3. The anchoring disruption molecule or AKAP mimic of claim 1 which comprises the following amino acid sequence:
L E Q Y A N Q L A D Q I I K E A T E,
or a variant of said sequence wherein said variant has a substitution at any one, two, three, four or five of positions X₁ to X₁₃ of the sequence, wherein said substitutions are selected from the group consisting of the substitutions:
X₁ is L, C, I, Y, V, W or F;
X₂ is K, R, H, E, D, C, V, A, I, Q, S, T or L;
X₃ is Q, D, E, A, S, I, F, K, R, L, M, T, G, N, W or V;
X₄ is N, D, E, S, A, M, K, R, G, T, W or Q;
X₅ is Q, D, E, M, F, I, S, K, R, C, W, Y, L or T;
X₆ is S, D, M, N, E, I, A, R, F, H, W, K, L, Y, Q or G;
X₇ is Q, D, E, I, K, R, T, V, F, N, S, L, W or M;
X₈ is I, A, S, L, D, E or V;
X₉ is K, C, D, E, R, A, M, T, W, H, Q, Y, L or S;
X₁₀ is E, D, R, Q or K;
X₁₁ is A, C, I, F, L, G, H or V;
X₁₂ is T, C, L, F, I, V, M, K, R or W;
X₁₃ is E, D, N, V, Y, K, A, F, G, H, I, Q, L, M, R, S, T or W; and
a peptidomimetic or analogue thereof.

4. The anchoring disruption molecule or AKAP mimic of claim 3 wherein said one or more substitutions are selected from:
X₁=F, Y, I, V, W or C;
X₂=C, D, R or K;
X₃=F, K, R, A, I, L, M, S, T, V, G, N, W, D or E;
X₄=K, R, G, T, S, W, D or E;
X₅=S, F, K, R, M, W, Y, D, E, L or T;
X₆=E, I, A, R, S, F, H, W, K, L, Y, M, N, Q or G;
X₇=K, R, F, N, S, T, V, L, M, W, I, D or E;
X₈=V;
X₉=D, E, L, S, R, A, M, T, W, Y;
X₁₀=D, R, K or Q;
X₁₁=I, V or C;
X₁₂=F, C, M, K, R, I or L; and
X₁₃=D, N, V, Y, G, H, I, Q, A, F, K, L, M, R, S, T or W.

5. The anchoring disruption molecule or AKAP mimic of claim 3 wherein said one or more substitutions are selected from the group consisting of
X₁=C, I, or F;
X₂=D, K, R, V, A, I, L, Q, S or T;
X₃=D, E, A, I, S or V
X₄=D, E, S, A, M or Q;
X₅=D, E, C, I, M or F;
X₆=G, S, E or M;
X₇=D, E, I or M;
X₈=V, D or E;
X₉=A, D, E, M, R, T, W or Y;
X₁₀=D
X₁₂=L, V, or W; and
X₁₃=D, R, K or W.

6. The anchoring disruption molecule or AKAP mimic of claim 3 wherein said substitutions are a single amino acid substitutions selected from the group consisting of
X₁=C, F, I or Y;
X₂=D;
X₃=D, E, A, K, M, R, S or T;
X₄=D, E, G or T;
X₅=D, E, K, L, M, R, S or T;
X₆=E, G, S, or N;
X₇=D, E, I, K, L or R;
X₉=D, E, A, M, R, T, W or Y;
X₁₀=D;
X₁₂=C or M; and
X₁₃ D, M, N, Q or T.

7. The anchoring disruption molecule or AKAP mimic of claim 3 wherein said substitutions are double amino acid substitutions selected from the group-consisting of:
a) when X₃ is A, either
X₄ is G;
X₅ is K, M or R;
X₇ is K, L or R; or
X₁₃ is M, N or Q; and
b) when X₁₃ is T, either
X₁ is F;
X₃ is A, K, E, M, R or S;
X₄ is T or G;
X₅ is M or R;
X₇ is L, R or K; or
X₉ is R; and
c) when X₁₃ is Q, either
X₃ is A, S, E, K, M, R or S;
X₄ is G;
X₅ is M, K or R; or
X₇ is K; and
d) when X₁₃ is M, either
X₃ is A, E, M, R, S or K;
X₄ is G;
X₅ is M, R or K;
X₇ is R; or
X₁₂ is C or M; and
e) when X₇ is K, either
X₃ is A, R, E, K or M;
X₄ is G;
X₅ is M;
X₉ is R;
X₁₂ is C or M; or
X₁₃ is Q, T, M or N; and
f) when X₅ is M, X₇ is K or X₁₃ is N, Q or T.

8. The PKA I anchoring disruption molecule on AKAP mimic of claim 1 which comprises the following amino acid sequence:
L K Q Y A N Q L A S Q V I K E A T E
or a variant thereof, in which said variant has a substitution at any one, two, three or four of positions X₁ to X₁₃ of said sequence, wherein said substitutions are selected from the group consisting of the following possible substitutions:
X₁ is L, C, I, Y, V, W or F;
X₂ is K, R, H E, D, C, V, A, I, Q, S, T or L;
X₃ is Q, D, E, A, S, I, F, K, R, L, M, T, G, N, W or V;
X₄ is N, D, E, S, A, M, K, R, G, T, W or Q;
X₅ is Q, D, E, M, F, I, S, K, R, C, W, Y, L or T;
X₆ is S, D, M, N, E, I, A, R, F, H, W, K, L, Y, Q or G;
X₇ is Q, D, E, I, K, R, T, V, F, N, S, L, W or M;
X₈ is I, A, S, L, D, E or V;
X₉ is K, C, D, E, R, A, M, T, W, H, Q, Y, L or S;
X₁₀ is E, D, R, Q or K;
X₁₁ is A, C, I, F, L, G, H or V;
X₁₂ is T, C, L, F, I, V, M, K, R or W;
X₁₃ is E, D, N, V, Y, K, A, F, G, H, I, Q, L, M, R, S, T or W; and
a peptidomimetic or analogue thereof.

9. The PKA I anchoring disruption molecule or AKAP mimic of claim 8 wherein said one or more substitutions are selected from:
(a) X₂=E; X₆=D; X₈=I;
(b) X₁ is F, Y, I, V, W or C; X₂ is C, D, R or K; X₃ is F, K, R, A, I, L, M, S, T, V, G, N, W, D or E; X₅ is S, F, K, R, M, W, Y, D, E, L or T; X₆ is E, I, A, R, S, F, H, W, K, L, Y, M, N, or G; X₇ is K, R, F, N, S, T, V, L, M, W, I, D or E; X₈ is V; X₉ is D, E, L, S, R, A, M, T, W, or Y; X₁₀ is D, R, K or Q; X₁₁ is I, V or C; X₁₂ is F, C, M, K, R, I or L; and X₁₃ is D, N, V, Y, G, H, I, Q, A, F, K, L, M, R, S, T or W;
(c) X₁ is C, I, or F; X₂ is D, K, R, V, A, I, L, Q, S or T; X₃ is D, E, A, I, S or V; X₅ is D, E, C, I, M or F; X₆ is G, S, E or M; X₇ is D, E, I or M; X₈ is V, D or E; X₉ is A, D, E, M, R, T, W or Y; X₁₀ is D; X₁₂ is L, V, or W; and X₁₃ is D, R, K or W; and
(d) X₁ is C, F, I or Y; X₂ is D; X₃ is D, E, A, K, M, R, S or T; X₅ is D, E, K, L, M, R, S or T; X₆ is E, G, S, or N; X₇ is D, E, I, K, L or R; X₉ is D, E, A, M, R, T, W or Y; X₁₀ is D; X₁₂ is C or M; and X₁₃ is D, M, N, Q or T.

10. The PKA I anchoring disruption molecule or AKAP mimic of claim 8 having a single amino acid substitution selected from the group consisting of
X₂=A, D, E, V, Q, S, I, L or T;
X₃=D, E, S or A;
X₄=D, E, A, S or M;
X₅=D, E or M;
X₆=D or E;
X₇=D or E;
X₈=I;
X₉=C;
X₁₀=D;
X₁₂=W or L; and
X₁₃=D.

11. The PKA I anchoring disruption molecule or AKAP mimic of claim 8 having double amino acid substitutions selected from the group consisting of:
(a) when X₂ is E or D, either
X₁ is I or F;
X₃ is A, E, D, S, I or V;
X₄ is A, E, D, S, M or Q;
X₅ is F, I, D, E, or M;
X₆ is M, D or E;
X₇ is D, E or M;
X₈ is I;
X₉ is R;
X₁₀ is D;
X₁₂ is L or W; or
X₁₃ is D, K or W;
(b) when X₆ is E or D, either
X₁ is I or F;
X₂ is A, E, D, S, I, V, L, Q or T;
X₃ is A, E, D, S, I or V;
X₄ is D or E;
X₅ is F, I, D, E, or M;
X₆ is M, D or E;
X₇ is M, D or E;
X₈ is I;
X₉ is R;
X₁₀ is D;
X₁₂ is L or W; or
X₁₃ is W, K or D; and
(c) when X₂ is V, X₃ is E or D.

12. The PKA I anchoring disruption molecule or AKAP mimic of claim 8 having triple amino acid substitutions selected from the group consisting of:
(a) when X₂ is E or D and X₅ is E or D, either
X₃ is S, D, E or A;
X₄ is E, D, or S;
X₈ is I;
X₁₂ is W or L;
(b) X₂ is T and X₄ and X₅ are both E or D;
(c) X₂ is A, X₄ is E or D and X₅ is E or D;
(d) X₂ is E or D, X₆ is D or E and either X₈ is I or X₁₃ is L;
(e) X₂ is E or D, X₆ is G and X₈ is I;
(f) X₂ is V, X₄ is E or D and X₅ is E or D;
(g) X₂ is D or E, X₆ is D or E and X₈ is I;
(h) X₂ is Q, X₄ is E or D and X₅ is E or D; and
(i) X₂ is E, X₆ is N and X₈ is I.

13. The PKA I anchoring disruption molecule or AKAP mimic of claim 8 having quadruple amino acid substitutions selected from the group consisting of:
(a) when X₂ is E or D, X₆ is D or E and X₈ is I, either
X₁ is C, F, I or Y;
X₃ is D, E, A, K, R, M, S or T;
X₄ is D, E, G or T;
X₅ is D, E, K, L, M, R, S or T;
X₇ is D, E, I, K, L or R;
X₉ is A, D, E, M, R, T, W or Y;
X₁₂ is C or M;
X₁₃ is N, Q or T; and
(b) X₂ is E or D, X₆ and X₇ are both D or E and X₁₂ is L.

14. A PKA I anchoring disruption molecules or AKAP mimic comprising the sequence as defined in claim 1 in which Y at the fourth position of said sequence is substituted with W and X₁ to X₁₃ are as defined in claim 1.

15. The anchoring disruption molecule or AKAP mimic of claim 1 having a 1-6-N or C terminal amino acid truncation.

16. The anchoring disruption molecule or AKAP mimic of claim 1 further comprising an amino acid sequence which assists cellular penetration of said anchoring disruption molecule or AKAP mimic or said molecule is in the form of a pro-drug.

17. The anchoring disruption molecule or AKAP mimic of claim 16 wherein said additional amino acid sequence is a polyarginine sequence, the HIV tat sequence or antennaepedia peptide.

18. The anchoring disruption molecule or AKAP mimic of claim 1 which associates with a molecule comprising amino acid residues 1 to 90 of PKA RI.

19. The anchoring disruption molecule or AKAP mimic of claim 1 which has a higher affinity for PKA RI than endogenous AKAPs.

20. The anchoring disruption molecule or AKAP mimic of claim 1 which has a higher affinity for PKA RI than for RII.

21. The anchoring disruption molecule or AKAP mimic of claim 20, which has a binding affinity for RI which is at least 50 times higher than its binding affinity for RII.

22. The anchoring disruption molecule or AKAP mimic of claim 1, which is a polypeptide or peptidomimetic of less than 100 amino acid residues in length, preferably 10 to 35 amino acid residues in length.

23. The anchoring disruption molecule or AKAP mimic of claim 1, wherein said molecule or mimic is a peptidomimetic or analogue of the sequence defined in claim 1.

24. An antibody or its antigen-binding fragment directed to the anchoring disruption molecule or AKAP mimic as defined in claim 1.

25. A nucleic acid molecule comprising a sequence encoding a polypeptide as defined in claim 1.

26. The nucleic acid molecule of claim 25, operably linked to an expression control sequence.

27. A method of preparing an anchoring disruption molecule or AKAP mimic as defined in claim 1, which comprises culturing a host cell containing a nucleic acid molecule comprising a sequence encoding said anchoring disruption molecule or AKAP mimic, under conditions whereby said anchoring disruption molecule or AKAP mimic is expressed and recovering said molecule polypeptide thus produced from the culture.

28. A method of altering the PKA type I signalling pathway in a cell by administration of an anchoring disruption molecule or AKAP mimic or a molecule encoding such an anchoring disruption molecule or AKAP mimic as defined in claim 1.

29. A method of treating or preventing a disease or condition in which abnormal PKA type I signalling is exhibited or which would benefit from a reduction or elevation in the levels of PKA type I signaling, comprising
administration of an anchoring disruption molecule or AKAP mimic or a molecule encoding such an anchoring disruption molecule or AKAP mimic as defined in claim 1 to a subject in need of such treatment.

30. A method of treating or preventing a disease or condition in which abnormal PKA type I signalling is exhibited or which would benefit from a reduction or elevation in the levels of PKA type I signaling comprising:
the step of obtaining a sample from an individual,
contacting cells from said sample with anchoring disruption molecule or AKAP mimic, or a nucleic acid molecule encoding an anchoring disruption molecule or AKAP mimic as defined in claim 1 and
administering said cells of said sample to the subject.

31. A pharmaceutical composition comprising an anchoring disruption molecule or AKAP mimic as defined in claim 1 or the encoding nucleic acid molecule, and one or more pharmaceutically acceptable excipients and/or diluents.

32. (canceled)

33. (canceled)

34. The method of claim 29 wherein said disease or condition is an immunosuppressive disorder or proliferative disease or autoimmune disease.

35. The method of claim 29, wherein said disease or condition is HIV infection, AIDS or common variable immunodeficiency or cancer, preferably colorectal carcinoma, pancreatic carcinoma, hepatocellular carcinoma, cancer mamma, ovarian cancer, non-small cell carcinoma of the lung, leukaemia, adenoma of the pituitary or thyroid or thyroid carcinoma.

36. The method of claim 29 wherein one or more additional active ingredients which are effective in treating the disorder or disease to be treated are administered.

37. The method of claim 36 wherein said additional active ingredients are selected from a cAMP antagonist, a COX-2 inhibitor, an NNRTIs (non-nucleoside reverse transcriptase inhibitors), an HIV protease inhibitor, a HAART (highly active antiretroviral therapy) medicament, a HIV vaccine or a cancer vaccine.

38. A method of identifying and/or isolating a PKA type I molecule comprising
contacting a sample containing said PKA molecule with an AKAP mimic as defined in claim 1, carrying a labelling means and capable of binding to PKA type I with high affinity and
assessing the level of said AKAP mimic which is bound and/or isolating said PKA to which said AKAP mimic is bound, wherein said level of AKAP mimic is indicative of the level of said PKA molecule in said sample.

39. A PKA type II anchoring disruption molecule or AKAP mimic, wherein said molecule or mimic is a polypeptide which comprises the following amino acid sequence:
LKQYANQLASQVIKEATE
in which E at position 15 is substituted with A, C, F, G, H, I, K, L, M, N, Q, R, S, T, V, W, or Y and/or E at position 18 is substituted with A, C, F, G, H, K, L, M or R and/or A at position 9 is substituted with V or a peptidomimetic or analogue thereof.

40. A method of altering the PKA type II signalling pathway in a cell by administration of an anchoring disruption molecule or AKAP mimic or a molecule encoding such an anchoring disruption molecule or AKAP mimic as defined in claim 39.

41. A method of treating a disease or conditions in which abnormal PKA type II signalling is exhibited or which would benefit from a reduction or elevation in the levels of PKA type II signaling comprising administering an anchoring disruption molecule or AKAP mimic, as defined in claim 39 to a subject in need of such treatment.
